(19) United States
(12) Patent Application Publication
(10) Pub. No.: US 2025/0066762 A1
(43) Pub. Date: Feb. 27, 2025

(54) SAMPLE HANDLING APPARATUS AND FLUID DELIVERY METHODS

(71) Applicant: 10x Genomics, Inc., Pleasanton, CA (US)

(72) Inventors: Liza S. Man, Pleasanton, CA (US); Augusto Manuel Tentori, Dublin, CA (US); Hanyoup Kim, Foster City, CA (US); Rajiv Bharadwaj, Pleasanton, CA (US); Ace George Santiago, Dublin, CA (US)

(21) Appl. No.: 18/830,249

(22) Filed: Sep. 10, 2024

Related U.S. Application Data
(63) Continuation of application No. PCT/US2023/014886, filed on Mar. 9, 2023.
(60) Provisional application No. 63/319,037, filed on Mar. 11, 2022.

(51) Int. Cl. C12N 15/10

(52) U.S. Cl. CPC C12N 15/1065

(57) ABSTRACT

A method of capturing analytes from a sample is provided. A first substrate including a sample is mounted on a first member of a sample handling device and mounting a second substrate including an array of capture probes on a second member of the sample handling device. A first reagent medium is applied to the first and/or second substrate and moving the first member and/or the second member to fluidically couple the sample and the array of capture probes via the first reagent medium. The sample and the array of capture probes are fluidically decoupled by moving the first member and/or the second member and a second reagent medium is applied to the first and/or second substrate and moving the first member and/or the second member to fluidically couple the sample and the array of capture probes via the second reagent medium. Related systems and apparatuses are also provided.

### CROSS REFERENCE TO RELATED APPLICATIONS

Pursuant to 35 U.S.C. § 119(e), this application is a continuation of International Application PCT/US2023/014886, with an international filing date of Mar. 9, 2023, which claims the benefit of U.S. Provisional Patent Applications Nos. 63/319,037 filed on Mar. 11, 2022, the contents of which are hereby incorporated by reference herein in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provide a lot of analyte data for single cells, but fail to provide information regarding the position of the single cell in a parent biological sample (e.g., tissue sample).

Analytes within the biological sample are generally released through disruption (e.g., permeabilization) of the biological sample. Various methods of delivering permeabilization reagents to the biological sample are described herein.

### SUMMARY

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Analytes within a biological sample are generally released through disruption (e.g., permeabilization) of the biological sample or other releasing means. Various methods of disrupting a biological sample are known, including permeabilization of the cell membrane of the biological sample. Described herein are methods of delivering a fluid to the biological sample, systems for sample analysis, and sample alignment methods. Also described herein are methods of delivering a fluid, including for example, a buffer or a permeabilization solutions having various detergents, buffers, proteases, and/or nucleases for different periods of time and at various temperatures.

In an aspect, a method of capturing analytes from a biological sample is provided. In an embodiment the method can include (a) mounting a first substrate on a first member of a sample handling device. The first substrate can include the biological sample disposed thereon. The method can also include (b) mounting a second substrate on a second member of the sample handling device. The second substrate can include an array of capture probes. A first capture probe of the array of capture probes can include a first spatial barcode sequence and a first capture domain and a second capture probe of the array of capture probes can include a second spatial barcode sequence and a second capture domain. The method can further include (c) applying a first reagent medium to the first substrate and/or the second substrate. The first reagent medium can be configured to release a first analyte from the biological sample. The method can also include (d) moving the first member and/or the second member such that a first area of the biological sample and the first capture probe are fluidically coupled via the first reagent medium, thereby releasing the first analyte from the first area of the biological sample. The released first analyte binds to the first capture domain. The method can further include (e) moving the first member and/or the second member to fluidically decouple the first area of the biological sample and the first capture probe. The method can also include (f) applying a second reagent medium to the first substrate and/or the second substrate. The second reagent medium configured to release a second analyte from the biological sample. The method can further include (g) moving the first member and/or the second member such that the first area of the biological sample and the second capture probe are fluidically coupled via the second reagent medium, thereby releasing the second analyte from the first area of the biological sample. The released second analyte binds to the second capture domain.

In some variations, one or more features disclosed herein including the following features may optionally be included in any feasible combination. For example, the first spatial barcode sequence and the second spatial barcode sequence can be identical. In another example, (d), (e), and (g) are performed with aid of a mechanical fixture provided within the sample handling device. The mechanical fixture configured to maintain alignment of first substrate and the second substrate such that the first area of the biological sample is vertically aligned with a first area of the array during (d), (e), and (g), the first area comprising the first capture probe and the second capture probe. In another example, the mechanical fixture is manually adjustable. In another example, the mechanical fixture can be adjustable via a controller of the sample handling device. In another example, the first spatial barcode sequence and the second spatial barcode sequence can be different.

In another example, a first area of the array can include the first capture probe and a second area of the array can include the second capture probe, and the method can further include acquiring, responsive to (d), first image data comprising a first overlay of the first area of the biological sample with the first area of the array. The method can further include acquiring, responsive to (g), second image data including a second overlay of the first area of the biological sample with the second area of the array within the capture domain. The method can further include registering the first image data and the second image data. The method can further include generating an aligned imaged based on the registering. The aligned image can include an overlay of the first area of the array with the second area of the array.

In another aspect a sample holder is provided. In an embodiment, the sample holder can include a first member including a first retaining mechanism configured to retain a first substrate. The first substrate can include a biological sample. The first retaining mechanism can include a first surface including an array area indicator for placing the first substrate such that the sample is overlaid with the array area indicator. The first substrate can also include a first recess formed into the first surface to a depth. The first recess can abut the array area indicator. The sample holder can also include a second retaining mechanism configured to retain a second substrate. The second substrate can include an array of capture probes and a reagent medium. The sample holder can also include an alignment mechanism configured to move the first member and/or the second member such that the biological sample or a portion thereof is vertically aligned and fluidically coupled via the reagent medium with the array when the first substrate and the second substrate are retained in the first and second members, respectively.

In some variations, one or more features disclosed herein including the following features may optionally be included in any feasible combination. For example, the second substrate can further include a spacer surrounding the array, such that when the biological sample or portion thereof is vertically aligned and fluidically coupled via the reagent medium with the array, the first substrate, the second substrate, and the spacer cam form a substantially enclosed chamber retaining the reagent medium. In another example, the first recess can be configured to be in fluid communication with a first channel formed into the first surface at a second depth. In another example, the second depth can be equal to or greater than the first depth. In another example, the first surface can further include a second recess formed into the first surface at a third depth, wherein the second recess abuts the array area indicator. In another example, the second recess can be configured to be in fluid communication with a second channel formed into the first surface at a fourth depth. In another example, the fourth depth can be equal to or greater than the third depth. In another example, the first or second channel can further include a reservoir configured to receive fluid from the channel. In another example, the first recess or second recess can include an elevated ridge configured to confine fluid within the first recess or second recess, respectively.

In another aspect, a sample holder is provided. In an embodiment, the sample holder can include a first member. The first member can include a retaining mechanism assembly within the first member. The retaining mechanism assembly can include a frame comprising a first surface, a second surface opposite the first surface, and an opening extending between the first surface and the second surface. The frame can be pivotably mounted within the first member and can further include a plurality of protrusions on the first surface arranged on opposite sides of the opening. The first member can also include a first retaining mechanism mounted on the second surface of the frame. The first retaining mechanism can be configured to retain a first substrate comprising a sample. The sample holder can also include a second member comprising a second retaining mechanism configured to retain a second substrate comprising an array of capture probes and a reagent medium. The sample holder can also include an alignment mechanism configured to move the first member and the second member when the first substrate and the second substrate are retained by the first and second retaining mechanisms, respectively, such that the biological sample is vertically aligned with the array of capture probes and the biological sample and the array of capture probes are fluidically coupled via the reagent medium.

In some variations, one or more features disclosed herein including the following features may optionally be included in any feasible combination. For example, the first retaining mechanism can be mounted to the second surface of the frame via a plurality of attachment components extending through respective holes of the plurality of recesses and into the first retaining mechanism. In another example, the plurality of protrusions can have a height configured to limit pivotal travel of the frame from +4 degrees to −4 degrees relative to a horizontal surface on which the sample holder is positioned. In another example, the retaining mechanism assembly can further include a plurality of brackets, a plurality of force transfer elements extending from the first surface of the frame, and a frame mount mated to the first surface of the frame via the plurality of brackets, the frame mount comprising a plurality of frame receiver holes at which the plurality of force transfer elements are received. In another example, the plurality of force transfer elements and the plurality of frame receiver holes are configured so as to balance a first compression force applied to the frame in a first vertical direction and a second compression force applied to the frame in a second vertical direction opposite the first vertical direction.

In another aspect, a sample holder is provided. In an embodiment, the sample holder can include a first member including a first retaining mechanism. The first retaining mechanism can include a plurality of magnets arranged around the periphery of the first retaining mechanism. The sample holder can also include a frame member configured to receive a first substrate comprising a biological sample. The frame member can be coupled with the first retaining mechanism via one or more magnets of the plurality of magnets. The sample holder can also include a second member including a second retaining mechanism configured to retain a second substrate. The second substrate can include comprising an array of capture probes and reagent medium. The sample holder can also include an alignment mechanism configured to move the first member and the second member when the first substrate and the second substrate are retained by the first and second retaining mechanisms, respectively, such that the biological sample is vertically aligned with the array of capture probes and the biological sample and the array of capture probes are fluidically coupled via the reagent medium.

In some variations, one or more features disclosed herein including the following features may optionally be included in any feasible combination. For example, the frame member can include a ferromagnetic material. In another example, the plurality of magnets includes 2-3, 4-6, 7-9, 10-12, or 13-15 magnets. In another example, the first retaining mechanism can further include a viewing window, and the frame member can be excluded from the viewing window when the frame member is coupled to the first retaining mechanism. In another example, the frame member can include a longitudinal opening extending along a length of the frame member, the first substrate passing through the longitudinal opening during insertion of the first substrate into the frame member and/or removal of the first substrate from the frame member. In another example, the frame member includes one or more clips. In another example, the frame member can be configured to be removably coupled to the first member retaining mechanism via one or more of the plurality of magnets.

In another aspect a sample holder is provided. In an embodiment, the sample holder can include a) a first member comprising a first retaining mechanism configured to retain a first substrate. The first substrate can include a biological sample. The sample holder can also include b) a second member including a second retaining mechanism configured to retain a second substrate. The second substrate can include an array of capture probes and reagent medium. The second retaining mechanism can include a first magnet and an alignment clip including a second magnet. The first magnet being vertically offset from the second magnet and the first magnet can exerts a repelling force against the second magnet when the second substrate is retained by the second retaining mechanism. The sample holder can also include c) an alignment mechanism configured to move the first member and the second member when the first substrate and the second substrate are retained by the first and second retaining mechanisms, respectively, such that the biological sample is vertically aligned with the array of capture probes and the biological sample and the array of capture probes are fluidically coupled via the reagent medium.

In some variations, one or more features disclosed herein including the following features may optionally be included in any feasible combination. For example, the first magnet can be offset from the second magnet by about 1-45 degrees relative to a pivot axis point of the alignment clip.

In another aspect a system for aligning a sample area with an array area is provided. In an embodiment, the system can include a sample holder. The sample holder can include a first member including a first retaining mechanism configured to retain a first substrate. The first substrate can include a sample disposed on the sample area. The sample holder can also include a second member including a second retaining mechanism configured to retain a second substrate received within the second retaining mechanism. The second substrate can include (i) an array of capture probes disposed on the array area and (ii) a reagent medium. The sample holder can also include an alignment mechanism configured to move the first member and/or the second member such that the biological sample or a portion thereof is vertically aligned and fluidically coupled via the reagent medium with the array when the first substrate and the second substrate are retained in the first and second members, respectively. The sample holder can also include an image capture device operatively coupled to the sample holder and configured to generate image data of the first substrate and the second substrate within the sample holder. The image capture device can include a phase contrast objective.

In some variations, one or more features disclosed herein including the following features may optionally be included in any feasible combination. For example, the system can further include a computing device communicatively coupled to the image capture device and to the sample holder, the computing device comprising a display, a data processor, and a non-transitory computer readable storage medium storing computer readable and executable instructions, which when executed can cause the data processor to move the first member and/or the second member such that the biological sample or a portion thereof is vertically aligned and fluidically coupled via the reagent medium with the array when the first substrate and the second substrate are retained in the first and second members, respectively.

In another example, the computer readable and executable instructions, when executed, can further cause the data processor to generate image data of the first substrate and the second substrate when the biological sample or a portion thereof is vertically aligned and fluidically coupled via the reagent medium.

In another aspect, a method is provided. The method can include mounting a first substrate on a first member of a sample holder. The first substrate can include a biological sample disposed thereon, and the sample holder can include a first member including a first retaining mechanism configured to retain the first substrate. The sample holder can also include a second member including a second retaining mechanism configured to retain a second substrate received within the second retaining mechanism. The second substrate can include (i) an array of capture probes disposed on the array area, and (ii) one or more array fiducials. The sample holder can also include an alignment mechanism configured to move the first member and/or the second member such that the biological sample or a portion thereof is vertically aligned and fluidically coupled via the reagent medium with the array when the first substrate and the second substrate are retained in the first and second members, respectively. The sample holder can also include an image capture device operatively coupled to the sample holder. The image capture device can be configured to generate image data of the first substrate and the second substrate within the sample holder. The image capture device can include a phase contrast objective. The method can also include mounting a second substrate onto the second member of the sample holder. The method can further include applying a reagent medium to the first substrate and/or the second substrate. The reagent medium can be configured to release one or more analytes from the biological sample. The method can also include using the alignment mechanism to move the first member and/or the second member such that the biological sample or a portion thereof is vertically aligned and fluidically coupled via the reagent medium with the array. The method can further include responsive to (d), using the image capture device to obtain an array image of (i) an overlay of the biological sample with the array and (ii) the one or more array fiducials.

In some variations, one or more features disclosed herein including the following features may optionally be included in any feasible combination. For example, the biological sample may not include an eosin stain. In another example, the method can further include receiving, by a data processor, array image data from the image capture device. The array image data can include the array image of the overlay of the biological sample with the array and the one or more array fiducials. The method can further include receiving, by the data processor, sample image data comprising a sample image of the biological sample. The method can further include registering, by the data processor, the sample image to the array image by aligning the sample image and the array image. The method can further include generating, by the data processor, an aligned image based on the registering. The aligned image can include an overlay of the sample image with the one or more array fiducials. The method can further include providing, by the data processor, the aligned image.

In another example, the aligned image can further include the one or more array fiducials aligned with the sample. In another example, the sample image can be of the sample on the first substrate. In another example, the one or more array fiducials can be located on the second substrate adjacent to, within, or distant from the array of capture probes configured on the second substrate. In another example, the one or more array fiducials can surround the array of capture probes. In another example, the first substrate can include one or more sample fiducials. In another example, the array image can be acquired such that a portion of the array overlays a portion of the sample based on a location of the one or more array fiducials and/or the one or more sample fiducials. In another example, the sample image can have a higher resolution than the array image.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 shows an exemplary spatial analysis workflow in accordance with some example implementations.

FIG. 2 depicts an example workflow for preparing the biological sample on a slide in accordance with some example implementations.

FIG. 3 is a schematic diagram depicting an exemplary permeabilization solution interaction between a tissue slide and a gene expression slide in a sandwich configuration in accordance with some example implementations.

FIG. 4 is a schematic diagram showing an example sample handling apparatus in accordance with some example implementations.

FIG. 5A depicts an example first member and an example second member in accordance with some example implementations.

FIG. 5B depicts an example of the first member coupled to the second member in accordance with some example implementations.

FIG. 5C depicts an example of the first member coupled to the second member including a coupling member coupled to the first substrate and the second substrate in accordance with some example implementations.

FIG. 6 is a diagram of an example first member and an example second member in accordance with some example implementations.

FIG. 7 depicts a diagram of a close-up bottom view of the first member coupled to the second member and an overlap area where the first substrate overlaps with the second substrate in accordance with some example implementations.

FIG. 8 depicts a front cross-sectional view of the example sample handling apparatus in accordance with some example implementations.

FIG. 9 is diagram of an example adjustment mechanism in accordance with some example implementations.

FIG. 10 is a perspective view of an example sample handling apparatus including an automated second member in accordance with some example implementations.

FIG. 11A is a perspective view of an example sample handling apparatus including a heater in accordance with some example implementations.

FIG. 11B is an exploded view of an example second member including the heater in accordance with some example implementations.

FIG. 11C is a graph of an example desired substrate (e.g., slide) temperature profile over time in accordance with some example implementations.

FIG. 12A is a perspective view of an example first member in accordance with some example implementations.

FIG. 12B is an exploded view of the example first member of FIG. 12A in accordance with some example implementations.

FIG. 13A is a perspective cross-section view of an example first member in accordance with some example implementations.

FIG. 13B is a perspective view of the example holder plate of FIG. 13A in accordance with some example implementations.

FIG. 13C is a perspective view of the example heat sink block of FIG. 13A in accordance with some example implementations.

FIG. 14A is a perspective view of an example sample handling apparatus in a closed position in accordance with some example implementations.

FIG. 14B is a perspective view of the example sample handling apparatus in an open position in accordance with some example implementations.

FIG. 15 is a perspective view of the example sample handling apparatus in accordance with some example implementations.

FIG. 16A is a perspective view of the example sample handling apparatus in accordance with some example implementations.

FIG. 16B is a front view of the example sample handling apparatus showing example dimensions of the apparatus in accordance with some example implementations.

FIG. 16C is a side view of the example sample handling apparatus showing example dimensions of the apparatus in accordance with some example implementations.

FIGS. 17A-17E depict an example workflow for an angled sandwich assembly in accordance with some example implementations.

FIG. 18A is a side view of the angled closure workflow in accordance with some example implementations.

FIG. 18B is a top view of the angled closure workflow in accordance with some example implementations.

FIG. 19A depicts the sample handling apparatus including a hinge and a sliding slot coupled to the first member in accordance with some example implementations.

FIG. 19B depicts the sample handling apparatus in an open configuration with a first substrate coupled to the first member and the second member retaining the second substrate in accordance with some example implementations.

FIGS. 20A-20E show an example workflow for an angled sandwich assembly in accordance with some example implementations.

FIGS. 21A-21C depict a workflow for loading slides into a sample handling apparatus for later alignment in accordance with some example implementations.

FIGS. 22A-22C depict a workflow for aligning the loaded slides of the sample handling apparatus in accordance with some example implementations.

FIG. 23 is a process flow diagram illustrating an example process for aligning a sample area with an array area according to some implementations of the current subject matter.

FIG. 24 is a diagram illustrating adjusting a location of the first substrate relative to the second substrate to align all or a portion of a sample area with an array area according to some implementations of the current subject matter.

FIG. 25 is a diagram illustrating an exemplary embodiments for adjusting a location of the first substrate relative to the second substrate based on an array area indicator configured within a sample holder according to some implementations of the current subject matter.

FIGS. 26A-26C are diagrams illustrating exemplary embodiments for indicating a sample area of a substrate according to some implementations of the current subject matter.

FIG. 27 illustrates an example process for automatically determining a sample area indicator based on a received image of the sample according to some implementations of the current subject matter.

FIGS. 28A-28B are diagrams illustrating an exemplary embodiment for receiving an input identifying a sample area indicator based on an image of a sample.

FIG. 29 illustrates an example process for automatically determining a sample area indicator based on a received plurality of video images according to some implementations of the current subject matter.

FIG. 30 is a process flow diagram illustrating an example process for automatically determining a sample area indicator responsive to determining an area of the sample according to some implementations of the current subject matter.

FIG. 31 is a process flow diagram illustrating an example process for determining a fiducial mark located on a first substrate according to some implementations of the current subject matter.

FIG. 32 is a process flow diagram illustrating an example process for identifying the sample area indicator based on a registered sample image according to some implementations of the current subject matter.

FIGS. 33A-33C depict a workflow for permeabilization of a sample of the sample handling apparatus in accordance with some example implementations.

FIGS. 34A-34C depict a workflow for image capture of the sandwiched slides of the sample handling apparatus during a permeabilization step in accordance with some example implementations.

FIG. 35 is a diagram of an example sample handling apparatus in accordance with some example implementations.

FIG. 36A shows an exemplary sandwich configuration in accordance with some example implementations.

FIG. 36B shows a fully formed sandwich creating a chamber formed from the one or more spacers, the first substrate, and the second substrate in accordance with some example implementations.

FIG. 36C depicts a top view of the configuration of FIG. 36B.

FIG. 37 depicts an example configuration for venting or removing bubbles from the chamber in accordance with some example implementations.

FIGS. 38A-38C show example configurations for that one or more spacers disposed on the first substrate and/or the second substrate in accordance with some example implementations.

FIGS. 39A-39E depict example configurations of the one or more spacers combined with one or more hydrophobic areas in accordance with some example implementations.

FIGS. 40A-40C depict a side view and a top view of an angled closure workflow for sandwiching a first substrate having a tissue sample and a second substrate in accordance with some example implementations.

FIGS. 41A-41L depict an example workflow for providing multiple sandwich closings in series in accordance with example implementations.

FIGS. 42A-42G depict an example workflow for providing multiple sandwich closings in parallel in accordance with example implementations.

FIG. 43 depicts a workflow for performing sample analysis in accordance with example implementations.

FIG. 44 depicts a table of example parameters of the workflow for performing sample analysis in accordance with example implementations.

FIG. 45 depicts a comparison between a non-sandwich control permeabilization step and a sandwich configuration permeabilization step in accordance with example implementations.

FIG. 46 is a diagram of an example system software architecture in accordance with some example implementations.

FIG. 47 is a diagram of a sample handling apparatus software building block in accordance with some example implementations.

FIG. 48A depicts a top view of one embodiment of troughs configured in a retaining mechanism (array substrate holder) of a sample handling apparatus described herein in accordance with some example implementations.

FIG. 48B depicts a top view of another embodiment of troughs configured in a retaining mechanism (sample substrate holder) of a sample handling apparatus described herein in accordance with some example implementations.

FIG. 49A depicts a top view of an embodiment of alignment marks configured on a retaining mechanism (sample substrate holder) of a sample handling apparatus described herein in accordance with some example implementations.

FIG. 49B depicts of another embodiment of alignment marks configured on a retaining mechanism (sample substrate holder) of a sample handling apparatus described herein in accordance with some example implementations.

FIG. 50 depicts an alignment clip configured on a retaining mechanism (array substrate holder) of a sample handling apparatus described herein in accordance with some example implementations.

FIG. 51 depicts one or more leveling mechanisms configured on a sample handling apparatus described herein in accordance with some example implementations.

FIGS. 52A-52C depict user interfaces associated with the one or more leveling mechanism configured on a sample apparatus described herein in accordance with some example implementations.

FIG. 53 depicts a hinge resistance mechanism configured on a sample handling apparatus described herein in accordance with some example implementations.

FIGS. 54A-54C depict a closure feedback mechanism configured on a sample handling apparatus described herein in accordance with some example implementations.

FIG. 55 depicts a gasket configured on a retaining mechanism (array substrate) of a sample handling apparatus described herein in accordance with some example implementations.

FIG. 56 depicts a recess in a retaining mechanism of a sample handling apparatus described herein in accordance with some example implementations.

FIG. 57 depicts a focus motor of a sample handling apparatus described herein in accordance with some example implementations.

FIG. 58 depicts an example of spatial clustering analysis and analysis of hippocampal transcript Hpca in accordance with some example implementations.

FIGS. 59A-59C depict an example fast closing speed condition for a sandwich assembly using angled closure in accordance with some example implementations.

FIGS. 60A-60C depict an example medium closing speed condition for a sandwich assembly using angled closure in accordance with some example implementations.

FIGS. 61A-61C depict an example slow closing speed condition for a sandwich assembly using angled closure in accordance with some example implementations.

FIGS. 62A-62B depict an exemplary fluid delivery scheme in accordance with some example implementations.

FIG. 63 depicts another exemplary fluid delivery scheme in accordance with some example implementations.

FIG. 64 depicts another exemplary fluid delivery scheme in accordance with some example implementations.

FIGS. 65A-65B depict another exemplary fluid delivery scheme in accordance with some example implementations.

FIG. 66 depicts another exemplary fluid delivery scheme in accordance with some example implementations.

FIG. 67 depicts another exemplary fluid delivery scheme in accordance with some example implementations.

FIG. 68 depicts an exemplary sandwich configuration where a first substrate, including a biological sample, and a second substrate are brought into proximity with one another in accordance with some example implementations.

FIG. 69 is a front view of an example sample handling apparatus in accordance with some example implementations.

FIG. 70 is a perspective view of an example sample handling apparatus in accordance with some example implementations.

FIG. 71 is a perspective view of a sample handling apparatus including multiple image capture devices in accordance with some example implementations.

FIG. 72 is a perspective view of a portion of a sample handling apparatus described herein including a light emitting diode (LED) in accordance with some example implementations.

FIG. 73 is a perspective view of a portion of a sample handling apparatus described herein including a spring configured to aid closure of the sample handling apparatus in accordance with some example implementations.

FIG. 74 is a perspective view of a portion of a sample handling apparatus described herein including a sensor in accordance with some example implementations.

FIG. 75 is a perspective view of a portion of a sample handling apparatus described herein including a motor in accordance with some example implementations.

FIG. 76 is a bottom view of a sample handling apparatus described herein including a fan in accordance with some example implementations.

FIG. 77A is an image illustrating an embodiment of a retaining mechanism configured for use in the sample handling apparatus described herein.

FIG. 77B is an image illustrating another embodiment of the retaining mechanism described in FIG. 77A.

FIG. 78 is a perspective view of an embodiment of a retaining mechanism and a frame member configured for use in the sample handling apparatus described herein accordance with some example implementations.

FIG. 79 is a perspective view of another embodiment of a retaining mechanism and a frame member configured for use in the sample handling apparatus described herein accordance with some example implementations.

FIG. 80 is a perspective view of the frame member of FIG. 78 in accordance with some example implementations.

FIG. 81 depicts an exemplary method of capturing analytes from a biological sample with aid of a sample handling device disclosed herein.

FIG. 82 depicts another exemplary method of capturing analytes from a biological sample with aid of a sample handling device disclosed herein.

FIG. 83 depicts another exemplary method of capturing analytes from a biological sample with aid of a sample handling device disclosed herein.

FIG. 84 depicts exemplary image data acquired via the exemplary method of FIG. 83.

### DETAILED DESCRIPTION

### I. Introduction

This disclosure describes apparatus, systems, methods, and compositions for spatial analysis of biological samples. This section describes certain general terminology, analytes, sample types, and preparative steps that are referred to in later sections of the disclosure. For example, the terms and phrases: spatial analysis, barcode, nucleic acid, nucleotide, probe, target, oligonucleotide, polynucleotide, subject, genome, adaptor, adapter, tag, hybridizing, hybridize, annealing, anneal, primer, primer extension, proximity ligation, nucleic acid extension, polymerase chain reaction (PCR) amplification, antibody, affinity group, label, detectable label, optical label, template switching oligonucleotide, splint oligonucleotide, analytes, biological samples, general spatial array-based analytical methodology, spatial analysis methods, immunohistochemistry and immunofluorescence, capture probes, substrates, arrays, analyte capture, partitioning, analysis of captured analytes, quality control, multiplexing, and/or the like are described in more detail in PCT Patent Application Publication No. WO2020/123320, the entire contents of which are incorporated herein by reference.

### Spatial Analysis

Tissues and cells can be obtained from any source. For example, tissues and cells can be obtained from single-cell or multicellular organisms (e.g., a mammal). The relationship between cells and their relative locations within a tissue sample may be critical to understanding disease pathology. Spatial transcriptomics technology may allow scientists to measure all the gene activity in a tissue sample and map where the activity is occurring. This technology and embodiments described herein may lead to new discoveries that may prove instrumental in helping scientists gain a better understanding of biological processes and disease.

Tissues and cells obtained from a mammal, e.g., a human, often have varied analyte levels (e.g., gene and/or protein expression) which can result in differences in cell morphology and/or function. The position of a cell or a subset of cells (e.g., neighboring cells and/or non-neighboring cells) within a tissue can affect, e.g., the cell's fate, behavior, morphology, and signaling and cross-talk with other cells in the tissue. Information regarding the differences in analyte levels (gene and/or protein expression) within different cells in a tissue of a mammal can also help physicians select or administer a treatment that will be effective and can allow researchers to identify and elucidate differences in cell morphology and/or cell function in the single-cell or multicellular organisms (e.g., a mammal) based on the detected differences in analyte levels within different cells in the tissue. Differences in analyte levels within different cells in a tissue of a mammal can also provide information on how tissues (e.g., healthy and diseased tissues) function and/or develop. Differences in analyte levels within different cells in a tissue of a mammal can also provide information of different mechanisms of disease pathogenesis in a tissue and mechanism of action of a therapeutic treatment within a tissue.

The spatial analysis methodologies herein provide for the detection of differences in an analyte level (e.g., gene and/or protein expression) within different cells in a tissue of a mammal or within a single cell from a mammal. For example, spatial analysis methodologies can be used to detect the differences in analyte levels (e.g., gene and/or protein expression) within different cells in histological slide samples, the data from which can be reassembled to generate a three-dimensional map of analyte levels (e.g., gene and/or protein expression) of a tissue sample obtained from a mammal, e.g., with a degree of spatial resolution (e.g., single-cell resolution).

Spatial heterogeneity in developing systems has typically been studied via RNA hybridization, immunohistochemistry, fluorescent reporters, or purification or induction of pre-defined subpopulations and subsequent genomic profiling (e.g., RNA-seq). Such approaches, however, rely on a relatively small set of pre-defined markers, therefore introducing selection bias that limits discovery. These prior approaches also rely on a priori knowledge. RNA assays traditionally relied on staining for a limited number of RNA species. In contrast, single-cell RNA-sequencing allows for deep profiling of cellular gene expression (including non-coding RNA), but the established methods separate cells from their native spatial context.

Spatial analysis methodologies described herein provide a vast amount of analyte level and/or expression data for a variety of multiple analytes within a sample at high spatial resolution, e.g., while retaining the native spatial context.

The binding of an analyte to a capture probe can be detected using a number of different methods, e.g., nucleic acid sequencing, fluorophore detection, nucleic acid amplification, detection of nucleic acid ligation, and/or detection of nucleic acid cleavage products. In some examples, the detection is used to associate a specific spatial barcode with a specific analyte produced by and/or present in a cell (e.g., a mammalian cell).

Capture probes can be, e.g., attached to a surface, e.g., a solid array, a bead, or a coverslip. In some examples, capture probes are not attached to a surface. In some examples, capture probes can be encapsulated within, embedded within, or layered on a surface of a permeable composition (e.g., any of the substrates described herein).

Non-limiting aspects of spatial analysis methodologies are described in WO 2011/127099, WO 2014/210233, WO 2014/210225, WO 2016/162309, WO 2018/091676, WO 2012/140224, WO 2014/060483, U.S. Pat. Nos. 10,002,316, 9,727,810, U.S. Patent Application Publication No. 2017/0016053, Rodriques et al., Science 363(6434):1463-1467, 2019; WO 2018/045186, Lee et al., Nat. Protoc. 10(3):442-458, 2015; WO 2016/007839, WO 2018/045181, WO 2014/163886, Trejo et al., PLoS ONE 14(2):e0212031, 2019, U.S. Patent Application Publication No. 2018/0245142, Chen et al., Science 348(6233):aaa6090, 2015, Gao et al., BMC Biol. 15:50, 2017, WO 2017/144338, WO 2018/107054, WO 2017/222453, WO 2019/068880, WO 2011/094669, U.S. Pat. Nos. 7,709,198, 8,604,182, 8,951,726, 9,783,841, 10,041,949, WO 2016/057552, WO 2017/147483, WO 2018/022809, WO 2016/166128, WO 2017/027367, WO 2017/027368, WO 2018/136856, WO 2019/075091, U.S. Pat. No. 10,059,990, WO 2018/057999, WO 2015/161173, and Gupta et al., Nature Biotechnol. 36:1197-1202, 2018, the entire contents of which are incorporated herein by reference and can be used herein in any combination. Further non-limiting aspects of spatial analysis methodologies are described herein.

Embodiments described herein may map the spatial gene expression of complex tissue samples (e.g., on tissue slides) with slides (e.g., gene expression slides) that utilize analyte and/or mRNA transcript capture and spatial barcoding technology for library preparation. A tissue (e.g., fresh-frozen, formalin-fixed paraffin-embedded (FFPE), or the like) may be sectioned and placed in proximity to a slide with thousands of barcoded spots, each containing millions of capture oligonucleotides with spatial barcodes unique to that spot. Once tissue sections are fixed, stained, and permeabilized, they release mRNA which binds to capture oligos from a proximal location on the tissue. A reverse transcription reaction may occur while the tissue is still in place, generating a cDNA library that incorporates the spatial barcodes and preserves spatial information. Barcoded cDNA libraries are mapped back to a specific spot on a capture area of the barcoded spots. This gene expression data may be subsequently layered over a high-resolution microscope image of the tissue section, making it possible to visualize the expression of any mRNA, or combination of mRNAs, within the morphology of the tissue in a spatially-resolved manner.

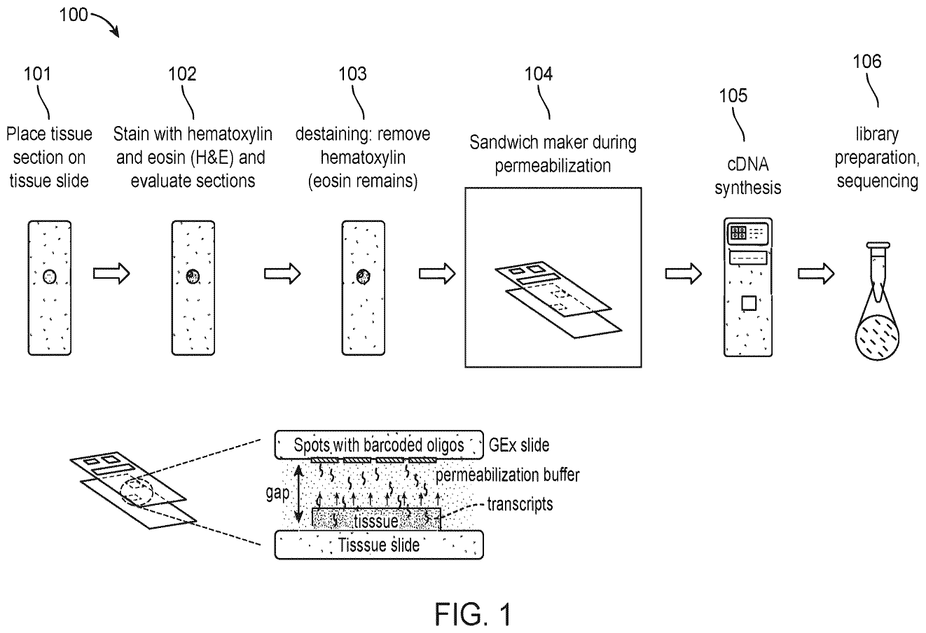

FIG. 1 shows an exemplary spatial analysis workflow 100 in accordance with some example implementations. The workflow 100 includes preparing a biological sample on a slide (e.g., a pathology slide) 101, fixing the sample, and/or staining 102 the biological sample for imaging. The stained sample can be then imaged on the slide using brightfield (to image the sample hematoxylin and eosin stain) and/or fluorescence (to image features) modalities. The imaging may include high resolution imaging (e.g., images that can disclose pathological and histological features). Optionally, at 103, the sample can be destained prior to permeabilization. At 104, a permeabilization solution may be applied to biological sample while the pathology slide is aligned in a “sandwich” configuration with a slide comprising a spatially barcoded array (e.g., on a GEx slide). The permeabilization solution allowing the analyte and/or mRNA transcripts to migrate away from the sample, diffuse across the permeabilization solution, and toward the array. The analyte and/or mRNA transcripts interacts with a capture probe on the spatially-barcoded array on the slide.

At 105, the capture probes can be optionally cleaved from the array, and the captured analytes can be spatially-barcoded by performing a reverse transcriptase first strand cDNA reaction. A first strand cDNA reaction can be optionally performed using template switching oligonucleotides. At 106, the first strand cDNA can be amplified (e.g., using polymerase chain reaction (PCR)), where the forward and reverse primers flank the spatial barcode and analyte regions of interest, generating a library associated with a particular spatial barcode. In some embodiments, the cDNA comprises a sequencing by synthesis (SBS) primer sequence. The library amplicons may be sequenced and analyzed to decode spatial information.

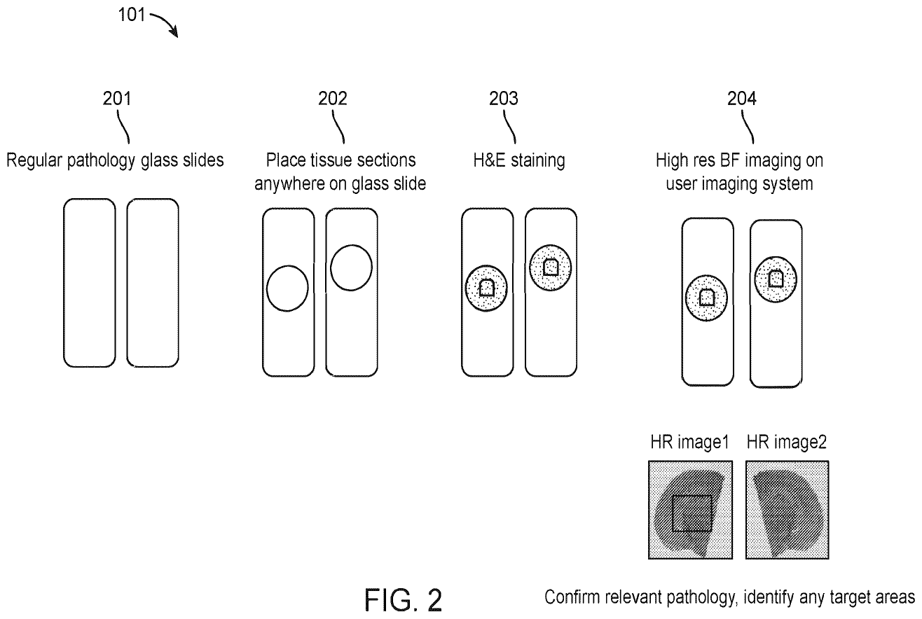

FIG. 2 depicts an example workflow 101 for preparing the biological sample on the slide (e.g., a pathology slide) in accordance with some example implementations. Preparing the biological sample on the slide may include selecting a pathology glass slide 201. The workflow 101 further includes placing tissue sections on the glass slide 202. Placing tissue sections on the glass slide may include placing the tissue anywhere on the glass slide including placing the tissue on or in relation to a fiducial disposed on the glass slide. The fiducial may include any marking to aid in placement of the tissue on the slide and/or aid in the alignment of the tissue slide relative to the gene expression slide. The workflow 101 further includes staining the tissue with hematoxylin and eosin 203 or another staining agent or method. The workflow 101 further includes imaging the tissue 204 on the slide using brightfield (to image the sample hematoxylin and eosin stain) or another imaging technique. The imaging may include high resolution imaging on a user imaging system. The imaging may allow the user to confirm the relevant pathology and/or identify any target areas for analysis.

Embodiments described herein relating to preparing the biological sample on the slide may beneficially allow a user to confirm pathology or relevant regions on a tissue section, to confirm selection of best or undamaged tissue sections for analysis, to improve array-tissue alignment by allowing placement anywhere on the pathology slide. Further, workflows for preparing the biological sample on the slide may empower user or scientists to choose what to sequence (e.g., what tissue section(s) to sequence).

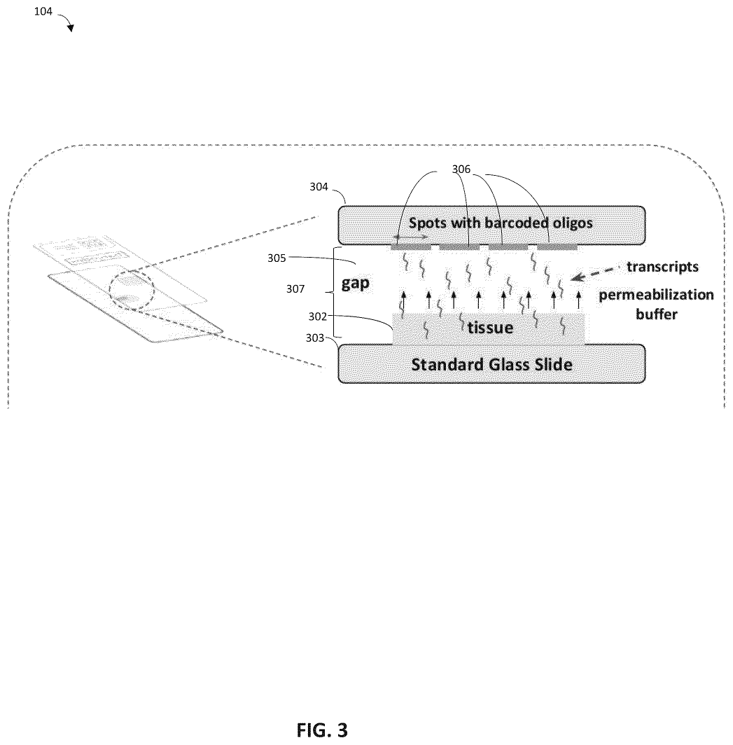

FIG. 3 is a schematic diagram depicting an exemplary sandwiching process (e.g., permeabilization solution interaction) 104 between a first substrate comprising a biological sample such as a tissue section (e.g., a tissue slide) and a second substrate comprising a spatially barcoded array, (e.g., a gene expression slide) in a sandwich configuration in accordance with some example implementations. During an exemplary sandwiching process, the first substrate is aligned with the second substrate, such that at least a portion of the biological sample is aligned with at least a portion of the array (e.g., aligned in a sandwich configuration). In the exemplary configuration, a sample (a tissue or biological sample) 302 is disposed on the pathology slide 303 and is sandwiched between the pathology slide 303 and a slide 304 (e.g., gene expression slide) that is populated with spatially-barcoded capture probes 306. As shown, the slide 304 is in a superior position to the pathology slide 303. In some embodiments, the pathology slide 303 may be positioned superior to the slide 304. When a permeabilization solution 305 is applied to a gap 307 between the pathology slide 303 and the slide 304, the permeabilization solution 305 creates a permeabilization buffer which permeabilizes or digests the sample 302 and the analytes and/or mRNA transcripts 308 of the sample (e.g., tissue sample) 302 may release, actively or passively migrate (e.g., diffuse) across the gap 307 toward the capture probes 306, and bind on the capture probes 306. In some embodiments, analyte capture agents that have bound to analytes in the sample (or portions of such analyte capture agents) may release, actively or passively migrate across the gap and bind on the capture probes. After the analytes (e.g., transcripts) 308 bind on the capture probes 306, an extension reaction (e.g., reverse transcription reaction) may occur, generating a spatially barcoded library. For example, in the case of mRNA transcripts, reverse transcription may occur, thereby generating a cDNA library associated with a particular spatial barcode. Barcoded cDNA libraries may be mapped back to a specific spot on a capture area of the capture probes 306. This gene expression data may be subsequently layered over a high-resolution microscope image of the tissue section ((e.g., taken at 204 of FIG. 2), making it possible to visualize the expression of any mRNA, or combination of mRNAs, within the morphology of the tissue in a spatially-resolved manner.

In some embodiments, the extension reaction can be performed separately from the sample handling apparatus described herein that is configured to perform the exemplary sandwiching process 104.

The sandwich configuration of the sample 302, the pathology slide 303 and the slide 304 may provide advantages over other methods of spatial analysis and/or analyte capture. For example, the sandwich configuration may reduce a burden of users to develop in house tissue sectioning and/or tissue mounting expertise. Further, the sandwich configuration may decouple sample preparation/tissue imaging from the barcoded array (e.g., spatially-barcoded capture probes 306) and enable selection of a particular region of interest of analysis (e.g., for a tissue section larger than the barcoded array). The sandwich configuration also beneficially enables spatial transcriptomics assays without having to place a tissue section 302 directly on the gene expression slide (e.g., slide 304) which may reduce cost and risk of mistakes/issues during sample preparation. The sandwich configuration may also provide an improvement of sensitivity and spatial resolution by vertically confining target molecules within the diffusion distance.

### II. Systems for Sample Analysis

The methods described above for analyzing biological samples, such as the sandwich configuration described above, can be implemented using a variety of hardware components. In this section, examples of such components are described. However, it should be understood that in general, the various steps and techniques discussed herein can be performed using a variety of different devices and system components, not all of which are expressly set forth.

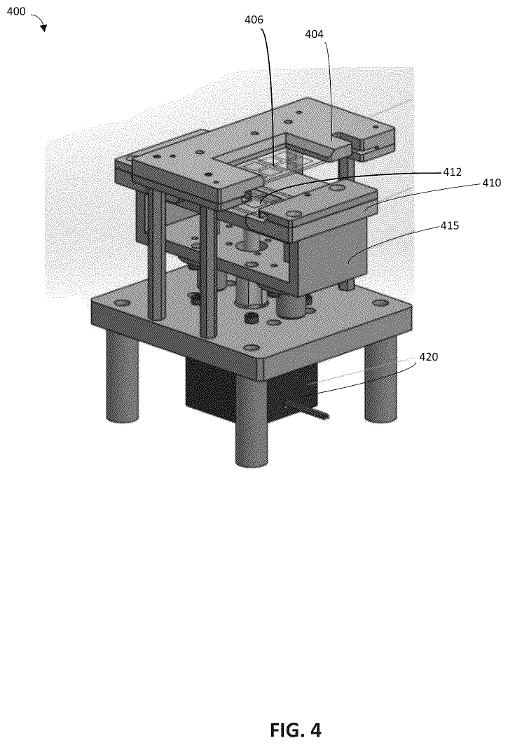

FIG. 4 is a schematic diagram showing an example sample handling apparatus 400 in accordance with some example implementations. Sample handling apparatus 400, also referred to as sample holder 400, includes a first member 404 that holds a first substrate 406 on which a sample 302 may be positioned. The first member 404 may include a first retaining mechanism configured to retain the first substrate 406 in a fixed position along an axis and disposed in a first plane. As shown, the sample handling apparatus 400 also includes a second member 410 that holds a second substrate 412. The second member 410 may include a second retaining mechanism configured to retain the second substrate 412 disposed in a second plane. The second substrate 412 may include a barcoded array (e.g., spatially-barcoded capture probes 306), as described above. As shown, the sample handling apparatus 400 also includes an adjustment mechanism 415 configured to move the second member 410. The adjustment mechanism 415 may be coupled to the second member 410 and includes a linear actuator 420 configured to move the second member 410 along a z axis orthogonal to the second plane. In some aspects, the adjustment mechanism 415 may be alternatively or additionally coupled to the first member 404.

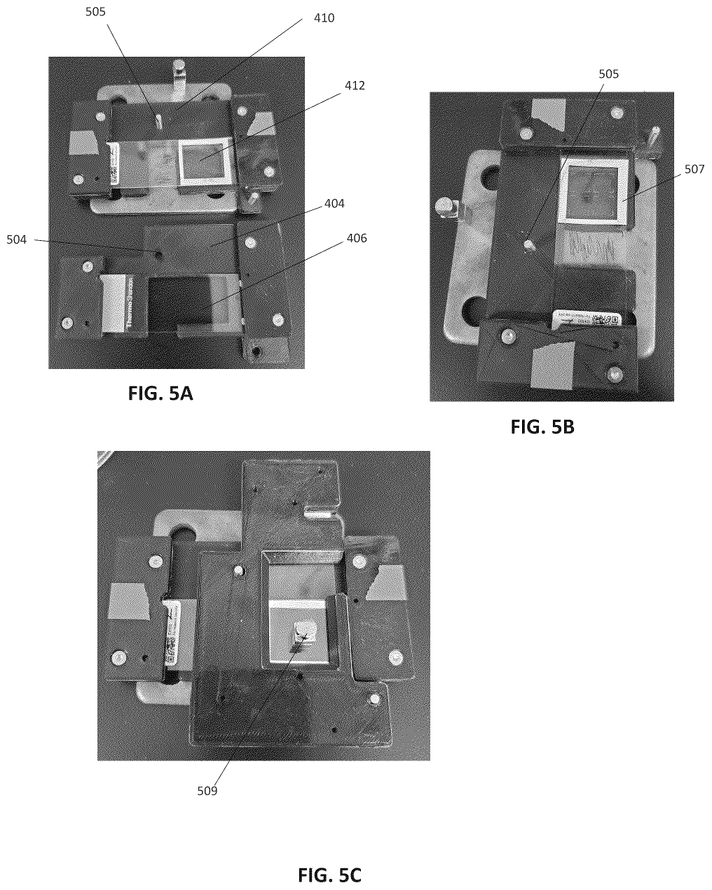

FIG. 5A depicts an example first member 404 and an example second member 410 in accordance with some example implementations. As shown, the second member 410 includes a pin 505. As further shown, the first member 404 includes an aperture 504. The aperture 504 may be sized and configured to mate with the pin 505. In some aspects, the adjustment mechanism 415 (not shown) may include the pin 505 and the aperture 504. The pin 505 and the aperture 504 mating may result in the first member 404 being aligned relative to the second member 410.

FIG. 5B depicts an example of the first member 404 coupled to the second member 410 in a sandwich configuration (e.g., via the pin 505 and the aperture 504) in accordance with some example implementations. As shown, the second substrate 412 includes a spacer 507 at least partially surrounding the barcoded array of the second substrate 412. The spacer 507 may be configured to contact and maintain a minimum spacing between the first substrate 406 and the second substrate 412. While the spacer 507 is shown as disposed on the second substrate 412, the spacer 507 may additionally or alternatively be disposed on the first substrate 406.

FIG. 5C depicts an example of the first member 404 coupled to the second member 410 in a sandwich configuration including a coupling member 509 coupled to the first substrate 406 and the second substrate 412 and configured to inhibit movement between the first substrate 406 and the second substrate 412 in accordance with some example implementations. In some aspects, the coupling member 509 includes a magnet that urges the first substrate 406 toward the second substrate 412 or vice versa (e.g., via a magnetic force).

FIG. 6 is a diagram of an example first member 604 and an example second member 410 in accordance with some example implementations. As shown in the left-hand side of FIG. 6, the first member 604 is coupled to the second member 410. The top right-hand side of FIG. 6 depicts the first member 604. As shown, the first member 604 is configured to retain two first substrates 406. As further shown, the two first substrates 406 are disposed substantially parallel to each other along a common plane (e.g., an xy-plane) within the first member 604. The first member includes a first retaining mechanism 608 configured to retain a first substrate 406. The first retaining mechanism 608 may include spring plungers configured to push the first substrate 406 to a position, may include a spring loaded clamp design configured to apply a force to the first substrate 406 to maintain contact between the first substrate 406 and the first member 604, or the like to retain the first substrate 406 in a position in the first member 604. The bottom-right hand side of FIG. 6 depicts the second member 410. The second member 410 includes a second retaining mechanism 609 configured to retain the second substrate 412. The second retaining mechanism 609 may include spring plungers configured to push the second substrate 412 to a position, may include a spring loaded clamp design configured to apply a force to the second substrate 412 to maintain contact between the second substrate 412 and the second member 410, or the like to retain the second substrate 412 in a position in the second member 410.

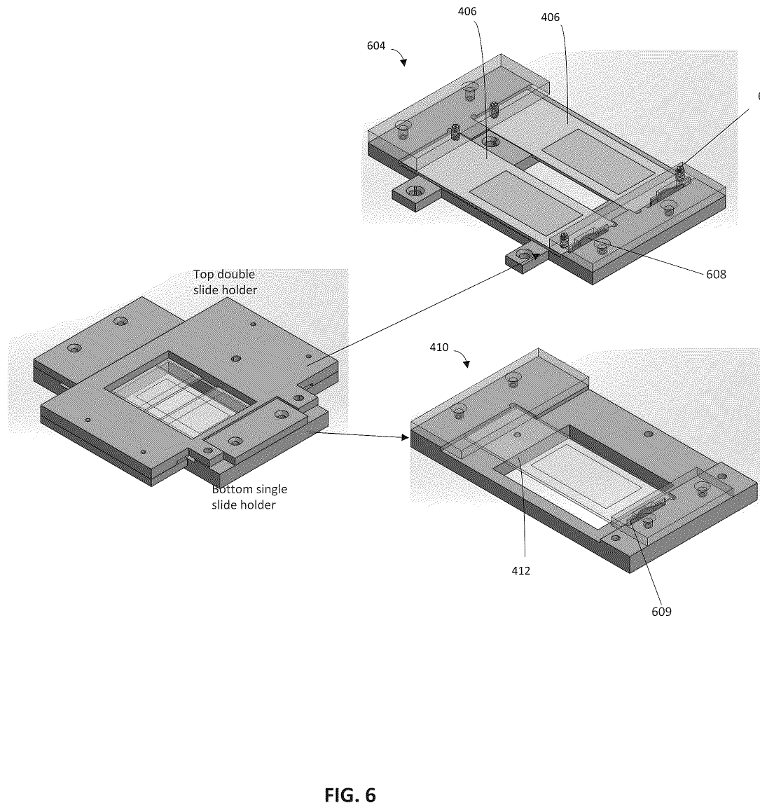

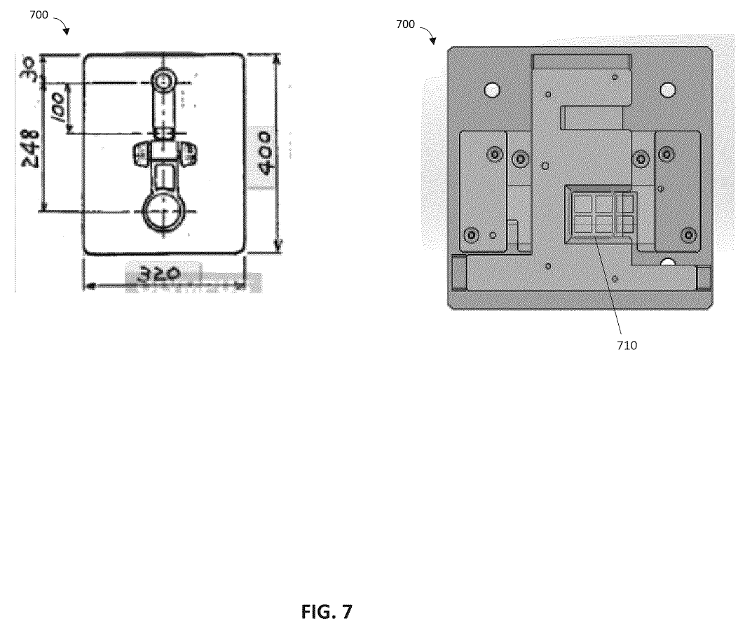

FIG. 7 depicts a diagram 700 of a close-up bottom view of the first member 404 coupled to the second member 410 and an overlap area 710 where the first substrate 406 overlaps with the second substrate 412 in accordance with some example implementations. The overlap may occur along an axis orthogonal to the first substrate 406 and/or orthogonal to the second substrate 412. In some aspects, a camera may capture an image of the overlap area 710 that may be used as part of the spatial analysis further described herein. In some embodiments, the diagram 700 depicts an assembly of the first member 404 coupled to the second member 410 having dimensions of 113 mm long and 112 mm wide, although other dimensions are possible.

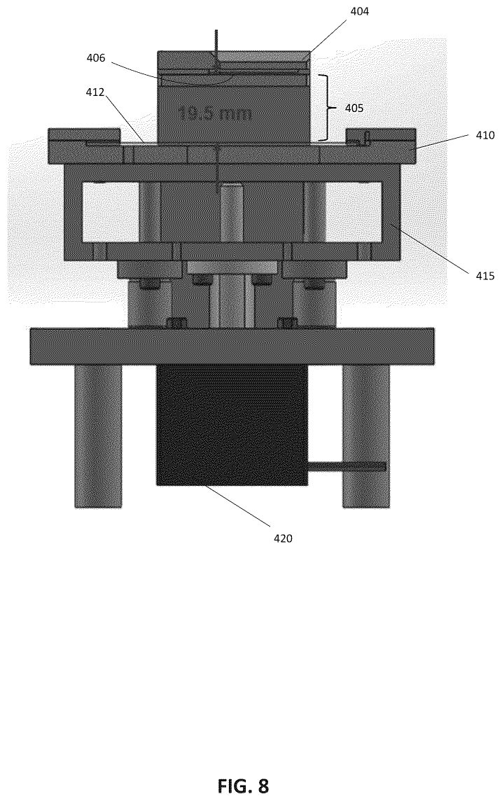

FIG. 8 depicts a front cross-sectional view of the sample handling apparatus 400 in accordance with some example implementations. As shown, the first member 404 and the second member 410 may be configured to maintain a separation distance 405 between the first substrate 406 and the second substrate 412. The separation distance 405 may be 19.5 mm in a home (e.g., default) or open position. In some aspects, the adjustment mechanism 415 may be configured to adjust the separation distance 405.

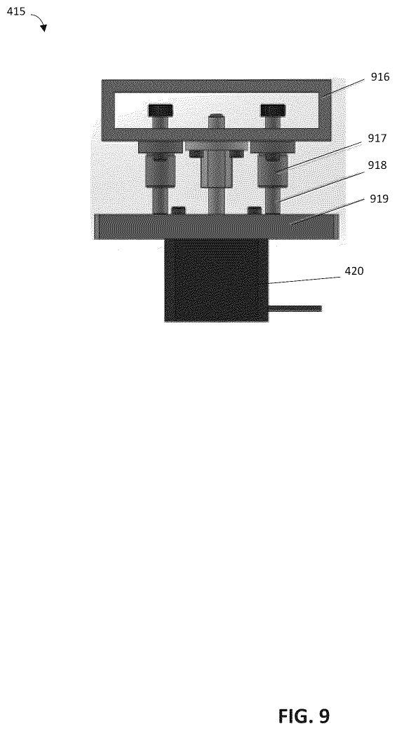

FIG. 9 is a diagram of an example adjustment mechanism 415 in accordance with some example implementations. The adjustment mechanism 415 may include a moving plate 916, a bushing 917, a shoulder screw 918, a motor bracket 919, and the linear actuator 420. The moving plate 916 may be coupled to the second member 410 and adjust the separation distance 405 along a z axis (e.g., orthogonal to the second substrate 412) by moving the moving plate 916 up in a superior direction toward the first substrate 406. The movement of the moving plate 916 may be accomplished by the linear actuator 420 configured to move the second member 410 along the axis orthogonal to the second plane at a velocity. The velocity may be controlled by a controller communicatively coupled to the linear actuator 420. For example, the velocity may be configured to move the moving plate between at least 0.1 mm/sec to 2 mm/sec. Further, the linear actuator may be configured to move the moving plate 916 with an amount of force (e.g., between 0.1-4.0 pounds of force). In some embodiments, the linear actuator may be configured to move the moving plate 916 with an amount of force that is between 0.1-100.0 pounds of force. For example, in some embodiments, the amount of force can be between 0.1 and 2.0 pounds of force, between 1.5 and 5.0 pounds of force, between 4.0 and 8.0 pounds of force, between 6.0 and 10.0 pounds of force, between 8.0 and 15.0 pounds of force, between 12.0 and 20.0 pounds of force, between 15.0 and 30.0 pounds of force, between 25.0 and 40.0 pounds of force, between 35.0 and 50.0 pounds of force, between 45.0 and 65.0 pounds of force, between 50.0 and 70.0 pounds of force, between 60.0 and 80.0 pounds of force, or between 70.0 and 100.0 pounds of force. In some embodiments, the force can be determined to provide improved resolution during imaging of the first substrate and/or the second substrate. The controller may be configured to adjust the velocity and/or the amount of force of the linear actuator 420 to accomplish a desired combination of velocity and force for the moving plate 916.

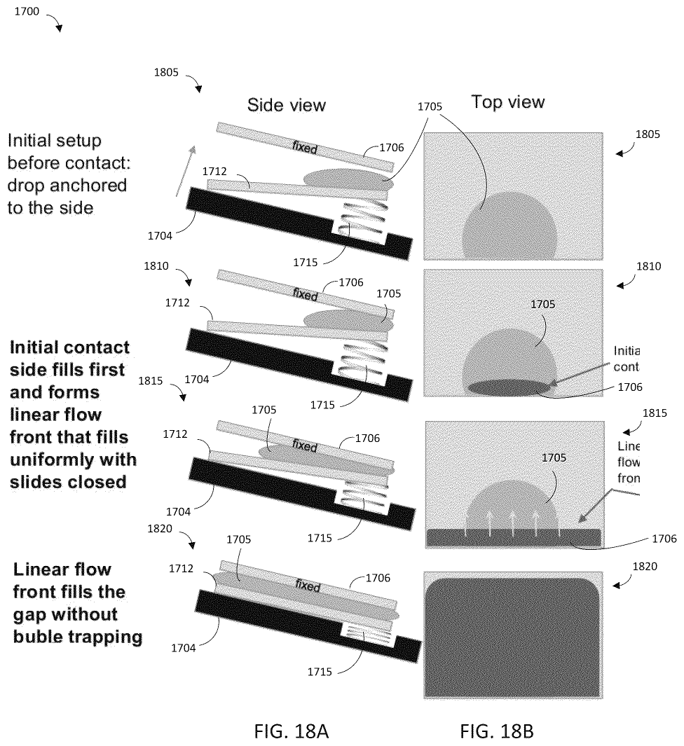

In some aspects, the velocity of the moving plate (e.g., closing the sandwich) may affect bubble generation or trapping within the permeabilization solution 305. In some embodiments, the closing speed is selected to minimize bubble generation or trapping within the permeabilization solution 305. In some embodiments, the closing speed is selected to reduce the time it takes the flow front of a reagent medium from an initial point of contact with the first and second substrate to sweep across the sandwich area (also referred to herein as “closing time”, see, e.g., FIG. 18B). In some embodiments, the closing speed is selected to reduce the closing time to less than about 1100 ms. In some embodiments, the closing speed is selected to reduce the closing time to less than about 1000 ms. In some embodiments, the closing speed is selected to reduce the closing time to less than about 900 ms. In some embodiments, the closing speed is selected to reduce the closing time to less than about 750 ms. In some embodiments, the closing speed is selected to reduce the closing time to less than about 600 ms. In some embodiments, the closing speed is selected to reduce the closing time to about 550 ms or less. In some embodiments, the closing speed is selected to reduce the closing time to about 370 ms or less. In some embodiments, the closing speed is selected to reduce the closing time to about 200 ms or less. In some embodiments, the closing speed is selected to reduce the closing time to about 150 ms or less. In some embodiments, the closing speed is selected to reduce the closing time to about 150-130 ms.

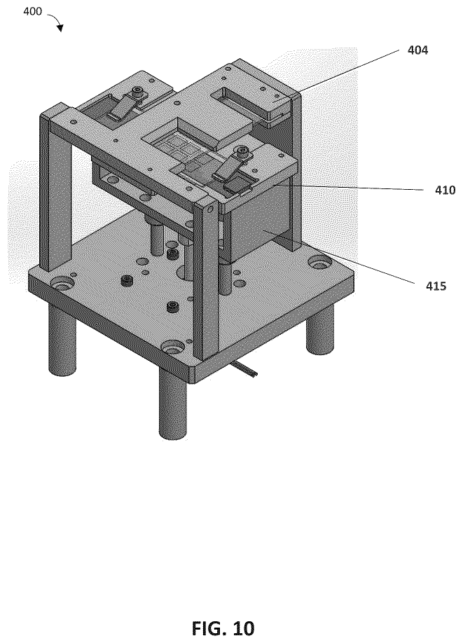

FIG. 10 is a perspective view of an example sample handling apparatus 400 including an automated second member 410 in accordance with some example implementations. As shown, the sample handling apparatus 400 includes the adjustment mechanism 415. The adjustment mechanism 415 may be automated such that one or more of the moving plate 916, the bushing 917, the shoulder screw 918, the motor bracket 919, and the linear actuator 420 may be controlled by a controller (not shown) communicatively coupled to the adjustment mechanism 415. The controller may be configured to adjust a position of the second member 410 relative to the first member 404 (e.g., separation distance 405). The first member 404 may be fixed with respect to one or more axes (e.g., the z axis).

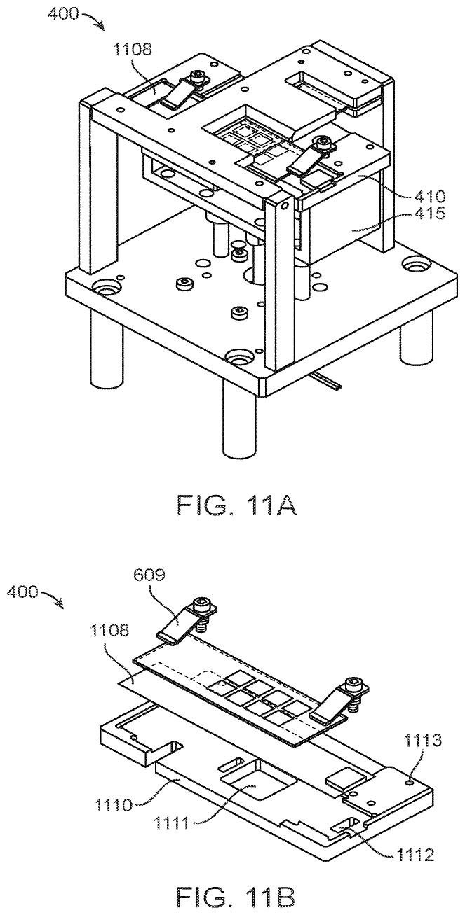

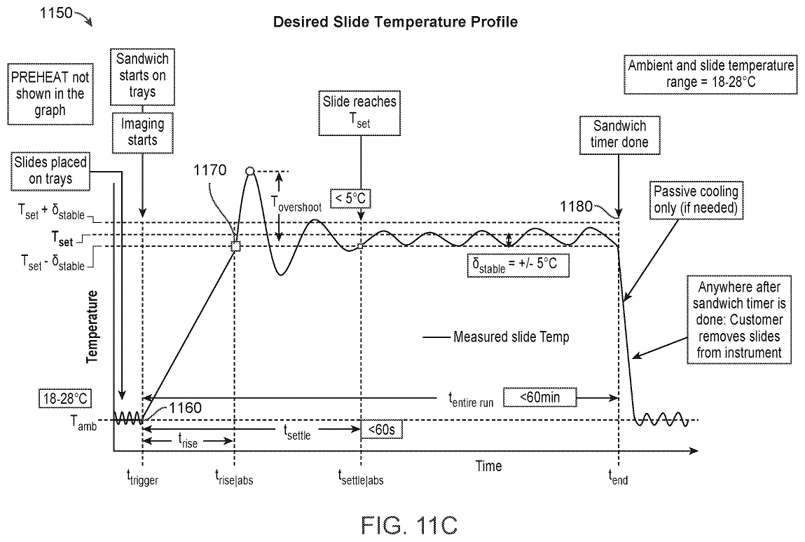

FIG. 11A is a perspective view of the example sample handling apparatus 400 including a heater 1108 in accordance with some example implementations. As shown, the sample handling apparatus 400 includes the heater 1108 as part of the second member 410.

FIG. 11B is an exploded view of an example second member 410 including the heater 1108 in accordance with some example implementations. As shown, the heater 1108 is positioned below or inferior to the second substrate 412 and above (superior to) the second member holder 1110. The heater 1108 may be configured to heat the second substrate 412 to a desired or target temperature. The second member holder 1110 includes a cutout window 1111 for the overlap area 710. The second member holder 1110 further includes an epoxy pocket 1112 for the heater 1108 and screw holes 1113 for the first substrate 406 and the second substrate 412 parallel alignment. As further shown, the second member 410 includes the second retaining mechanism 609. The second retaining mechanism 609 may include a swing clamp, a spring-loaded clamp, or the like to retain the second substrate 412 in a position within the second member 410. In some embodiments, the heater 1108 can be configured within a bracket. For example, a bracket can be coupled to the second member holder 1110. The bracket can secure the heater 1108 to the second member holder 1110. In some embodiments, at least a portion of the heater 1108 can be configured within a portion of the second member holder 1110.

FIG. 11C is a graph 1150 of an example desired substrate (e.g., slide) temperature profile over time in accordance with some example implementations. As shown in the graph 1150, the temperature of the slide may hover close to an ambient temperature (e.g., between 18-28° C.) until a trigger time 1160 (e.g., when imaging starts or when sandwiching of the substrates starts). After the trigger time 1160, the heater 1108 may heat the slide and the slide temperature may rise linearly until the slide temperature reaches a threshold temperature to the desired slide temperature at 1170. After the threshold temperature is reached, the slide temperature may fluctuate sinusoidally around the desired slide temperature, Tₛₑₜ, and may settle within a threshold amplitude around the desired temperature Tₛₑₜ. At 1180, the sandwich timer may complete and the slide temperature may begin to lower and return to the ambient temperature. In some aspects, the desired temperature may be based on the tissue sample 302, the permeabilization solution 305, a starting temperature of the first substrate or the second substrate, or the like.

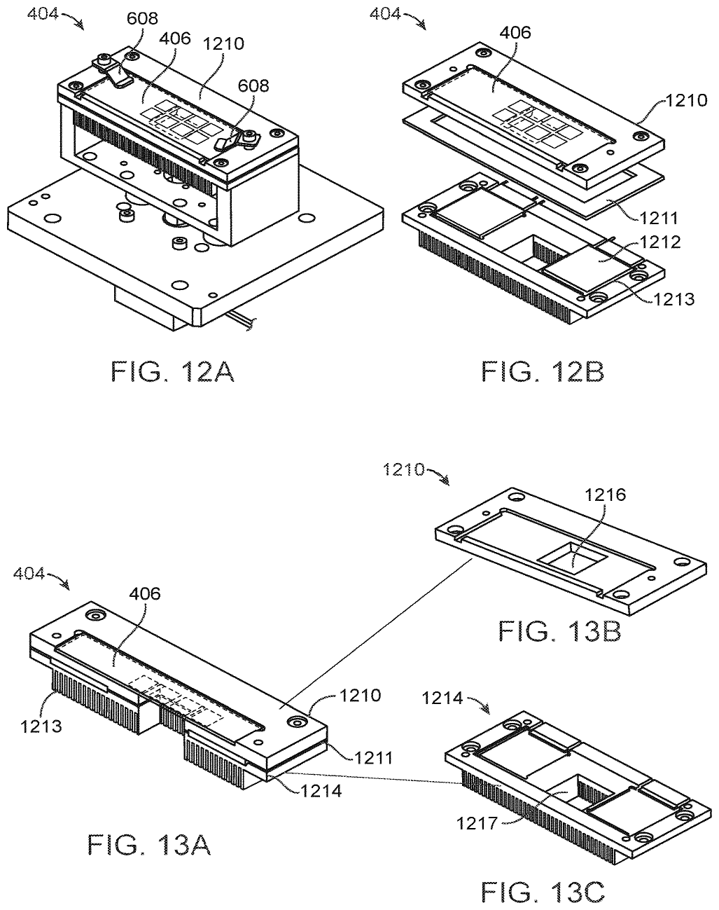

FIG. 12A is a perspective view of an example first member 404 in accordance with some example implementations. As shown, the first member 404 includes a holder plate 1210 and the first retaining mechanism 608 retaining the first substrate 406 within the first member 404.

FIG. 12B is an exploded view of the example first member 404 of FIG. 12A in accordance with some example implementations. As shown, the first member 404 includes the holder plate 1210, an insulation gasket 1211, a thermal pad 1212, and a thermoelectric cooler (TEC) 1213. The holder plate 1210 may be configured to receive and retain the first substrate 406. The insulation gasket 1211, the thermal pad 1212, and/or the TEC 1213 may be configured to adjust and/or maintain a desired or target temperature for the first substrate 406.

FIG. 13A is a perspective cross-section view of an example first member 404 in accordance with some example implementations. As shown, the first member 404 of FIG. 13A includes the holder plate 1210, the insulation gasket 1211, the TEC 1213, and a heat sink block 1214.

FIG. 13B is a perspective view of the example holder plate 1210 of FIG. 13A in accordance with some example implementations. As shown, the holder plate 1210 includes a cutout window 1216 for the overlap area 710.

FIG. 13C is a perspective view of the example heat sink block 1214 of FIG. 13A in accordance with some example implementations. As shown, the heat sink block 1214 includes a cut out window 1217 for the overlap area 710.

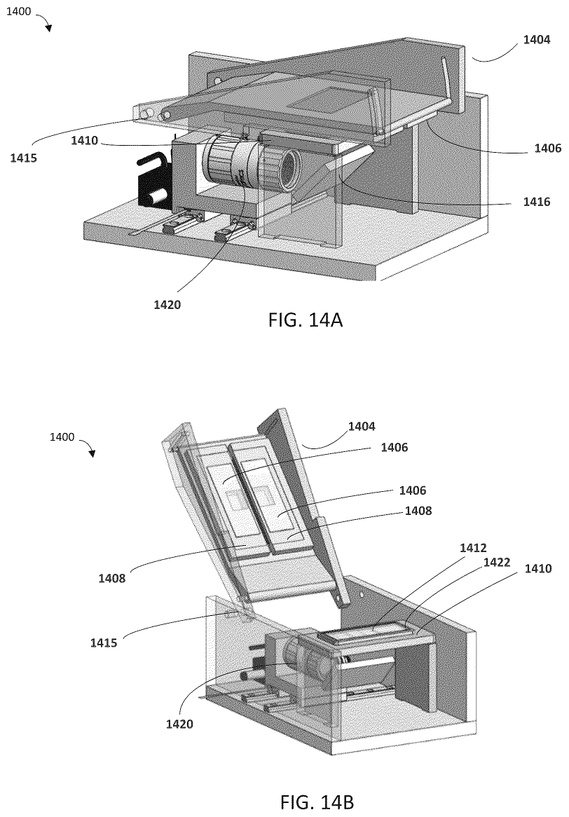

FIG. 14A is a perspective view of an example sample handling apparatus 1400 in a closed position in accordance with some example implementations. As shown, the sample handling apparatus 1400 includes a first member 1404, a second member 1410, an image capture device 1420, a first substrate 1406, a hinge 1415, and a mirror 1416. Although not shown in FIG. 14A, the example sample handling apparatus 1400 may include a first retaining mechanism 1408. The hinge 1415 may be configured to allow the first member 1404 to be positioned in an open or closed configuration by opening and/or closing the first member 1404 in a clamshell manner along the hinge 1415. In some embodiments, the mirror 1416 can be mounted to a platform via one or more springs. The springs can be secured within the platform via one or more spring pins, or dowels. The springs can include loops at opposite ends of the spring to couple to the spring pins or dowels configured within the mirror platform.

FIG. 14B is a perspective view of the example sample handling apparatus 1400 in an open position in accordance with some example implementations. As shown, the sample handling apparatus 1400 includes one or more first retaining mechanisms 1408 configured to retain one or more first substrates 1406. In the example of FIG. 14B, the first member 1404 is configured to retain two first substrates 1406 (e.g., within the first retaining mechanism 1408), however the first member 1404 may be configured to retain more or fewer first substrates 1406. As further shown in FIG. 14B, the sample handling apparatus can include a second retaining mechanism 1422. The second retaining mechanism 1422 can be configured on the second member 1410 and can receive and secure the second substrate 1412 to the second member 1410.

In some aspects, when the sample handling apparatus 1400 is in an open position (as in FIG. 14B), the first substrate 1406 and/or the second substrate 1412 may be loaded and positioned within the sample handling apparatus 1400 such as within the first member 1404 and the second member 1410, respectively. As noted, the hinge 1415 may allow the first member 1404 to close over the second member 1410 and form a sandwich configuration (e.g., the sandwich configuration shown in FIG. 3).

In some aspects, after the first member 1404 closes over the second member 1410, an adjustment mechanism (not shown) of the sample handling apparatus 1400 may actuate the first member 1404 and/or the second member 1410 to form the sandwich configuration for the permeabilization step (e.g., bringing the first substrate 1406 and the second substrate 1412 closer to each other and within a threshold distance for the sandwich configuration). The adjustment mechanism may be configured to control a speed, an angle, or the like of the sandwich configuration.

In some embodiments, the tissue sample (e.g., sample 302) may be aligned within the first member 1404 (e.g., via the first retaining mechanism 1408) prior to closing the first member 1404 such that a desired region of interest of the sample 302 is aligned with the barcoded array of the gene expression slide (e.g., the slide 304), e.g., when the first and the second substrates are aligned in the sandwich configuration. Such alignment may be accomplished manually (e.g., by a user) or automatically (e.g., via an automated alignment mechanism). After or before alignment, spacers may be applied to the first substrate 1406 and/or the second substrate 1412 to maintain a minimum spacing between the first substrate 1406 and the second substrate 1412 during sandwiching. In some aspects, the permeabilization solution (e.g., permeabilization solution 305) may be applied to the first substrate 1406 and/or the second substrate 1412. The first member 1404 may then close over the second member 1410 and form the sandwich configuration. Analytes and/or mRNA transcripts 308 May be captured by the capture probes 306 and may be processed for spatial analysis.

In some embodiments, during the permeabilization step, the image capture device 1420 may capture images of the overlap area (e.g., overlap area 710) between the tissue 302 and the capture probes 306. If more than one first substrates 1406 and/or second substrates 1412 are present within the sample handling apparatus 1400, the image capture device 1420 may be configured to capture one or more images of one or more overlap areas 710.

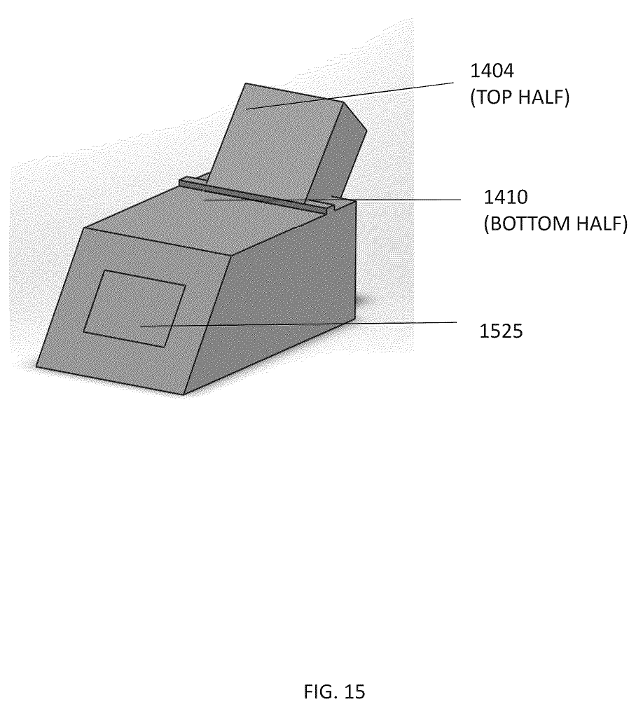

FIG. 15 is a perspective view of the example sample handling apparatus 1400 in accordance with some example implementations. As shown, the sample handling apparatus 1400 is in an open position with the first member 1404 disposed above (superior to) the second member 1410. As noted above, the first member 1404 and/or the second member 1410 may be configured to hold one or more substrates (e.g., first substrates 1406 and/or second substrates 1412, respectively). The sample handling apparatus 1400 further includes a user interface 1525. The user interface 1525 may include a touchscreen display for displaying information relating to the sample handling apparatus and receiving user input controls for controlling aspects or functions of the sample handling apparatus 1400.

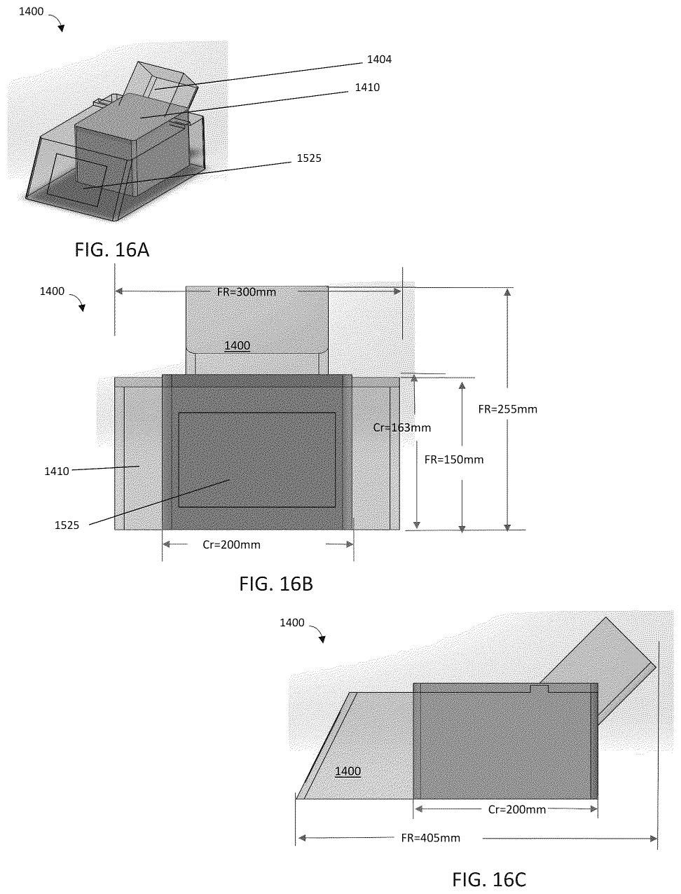

FIG. 16A is a perspective view of the example sample handling apparatus 1400 in accordance with some example implementations.

FIG. 16B is a front view of the example sample handling apparatus 1400 showing example dimensions of the apparatus 1400 in accordance with some example implementations. As shown, the sample handling apparatus may have a width of 300 mm and a height of 255 mm, although other dimensions are possible. The second member 1410 may have a height of 150 mm and a width of 300 mm, although other dimensions are possible.

FIG. 16C is a side view of the example sample handling apparatus 1400 showing example dimensions of the apparatus 1400 in accordance with some example implementations. As shown, the sample handling apparatus may have a depth of 405 mm, although other dimensions are possible.

### III. Fluid Delivery Methods and Kits

Analytes within a biological sample are generally released through disruption (e.g., permeabilization, digestion, etc.) of the biological sample or may be released without disruption. Various methods of permeabilizing (e.g., any of the permeabilization reagents and/or conditions described herein) a biological sample are described herein, including for example including the use of various detergents, buffers, proteases, and/or nucleases for different periods of time and at various temperatures. Additionally, various methods of delivering fluids (e.g., a buffer, a permeabilization solution) to a biological sample are described herein including the use of a substrate holder (e.g., sandwich assembly, sandwich configuration, as described herein)

### Fluid Delivery Methods and Kits

Provided herein are methods for delivering a fluid to a biological sample disposed on an area of a first substrate and an array disposed on a second substrate.

In some embodiments and with reference to FIG. 3, the sandwich configuration described herein between a tissue sample slide (e.g., pathology slide 303) and a gene expression slide (e.g., slide 304 with barcoded capture probes 306) may require the addition of a liquid reagent (e.g., permeabilization solution 305 or other target molecule release and capture solution) to fill a gap (e.g., gap 307). It may be desirable that the liquid reagent be free from air bubbles between the slides to facilitate transfer of target molecules with spatial information. Additionally, air bubbles present between the slides may obscure at least a portion of an image capture of a desired region of interest. Accordingly, it may be desirable to ensure or encourage suppression and/or elimination of air bubbles between the two slides during a permeabilization step (e.g., step 104).

In some aspects, it may be possible to reduce or eliminate bubble formation between the slides using a variety of filling methods and/or closing methods. For example, during the sandwiching of the two slides (e.g., the pathology slide 303 and the slide 304) it may be possible to provide an angled closure of the slides to suppress or eliminate bubble formation.

Workflows described herein include contacting a drop of the liquid reagent disposed on a first substrate (e.g., the first substrate 406, 1406, or the like) or a second substrate (e.g., the second substrate 412, 1412, or the like) with at least a portion of a first substrate (e.g., the first substrate 406, 1406, or the like) or second substrate (e.g., the second substrate 412, 1412, or the like), respectively. In some embodiments, the contacting comprises bringing the two substrates into proximity such that the sample on the first substrate is aligned with the barcode array of capture probes on the second substrate. In some instances, the contacting is achieved by arranging the first substrate and the second substrate in an angled sandwich assembly as described herein.

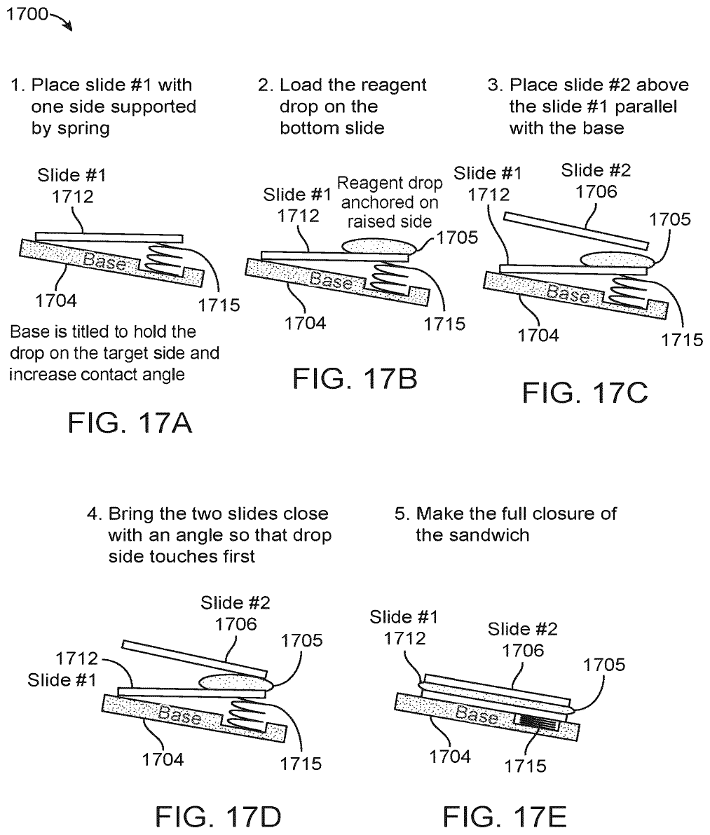

FIGS. 17A-17E depict an example workflow 1700 for an angled sandwich assembly in accordance with some example implementations. As shown in FIG. 17A, a slide 1712 (e.g., slide 304, second substrate 412, second substrate 1412, or the like) may be positioned and placed on a base 1704 with a side of the slide 1712 supported by a spring 1715. The spring 1715 may extend from the base 1704 in a superior direction and may be configured to dispose the slide 1712 along a plane angled differently than the base 1704. The angle of the slide 1712 may be such that a drop (e.g., drop 1705) placed on the surface of the slide 1712 will not fall off the surface (e.g., due to gravity). The angle may be determined based on a gravitational force versus any surface force to move the drop away from and off the slide 1712. The base 1704 may include the holder plate 1210 of FIG. 12A-13B, the second member holder 1110 of FIG. 11B, or the like.

FIG. 17B depicts a drop 1705 of liquid reagent placed on the slide 1712. As shown, the drop 1705 is located on the side of the slide 1712 contacting the spring 1715 and is located in proximity and above (superior to) the spring 1715.

As shown in FIG. 17C, a second slide 1706 (e.g., the slide 304, the first substrate 406, the first substrate 1406, or the like) may be positioned above (superior to) the slide 1712 and at an angle substantially parallel with the base 1704. For example, a first member (e.g., first member 404, first member 1404, or the like) of a sample handling apparatus (e.g., the sample handling apparatus 400, the sample handling apparatus 1400, or the like) may be configured to retain the slide 1706 at the angle substantially parallel to the base 1704.

As shown in FIG. 17D, slide 1706 may be lowered toward the slide 1712 such that a dropped side of the slide 1706 contacts the drop 1705 first. In some aspects, the drop side of the slide 1706 may urge the drop 1705 toward the opposite side of the slide 1706. In some aspects, the slide 1712 may be moved upward toward the slide 1706 to accomplish the contacting of the dropped side of the slide 1706 with the drop 1705.

FIG. 17E depicts a full sandwich closure of the slide 1706 and the slide 1712 with the drop 1705 positioned between the two sides. In some aspects and as shown, as the slide 1706 is lowered onto the drop 1705 and toward the slide 1712 (or as the slide 1712 is raised up toward the slide 1706), the spring 1715 may compress and the slide 1712 may lower to the base 1704 and become substantially parallel with the slide 1706.

FIG. 18A is aside view of the angled closure workflow 1700 in accordance with some example implementations. FIG. 18B is a top view of the angled closure workflow 1700 in accordance with some example implementations. As shown at step 1805 and in accordance with FIGS. 17C-D, the drop 1705 is positioned to the side of the slide 1712 contacting the spring 1715.

At step 1810, the drop side of the angled slide 1706 contacts the drop 1705 first. The contact of the slide 1706 with the drop 1705 may form a linear or low curvature flow front that fills uniformly with the slides closed.

At step 1815, the slide 1706 is further lowered toward the slide 1712 (or the slide 1712 is raised up toward the slide 1706) and the dropped side of the slide 1706 may contact and may urge the liquid reagent toward the side opposite the dropped side and creating a linear or low curvature flow front that may prevent or reduce bubble trapping between the slides. As further shown, the spring 1715 may begin to compress as the slide 1706 is lowered.

At step 1820, the drop 1705 of liquid reagent fills the gap (e.g., the gap 307) between the slide 1706 and the slide 1712. The linear flow front of the liquid reagent may form by squeezing the drop 1705 volume along the contact side of the slide 1712 and/or the slide 1706. Additionally, capillary flow may also contribute to filling the gap area. As further shown in step 1820, the spring 1715 may be fully compressed such that the slide 1706, the slide 1712, and the base 1704 are substantially parallel to each other.

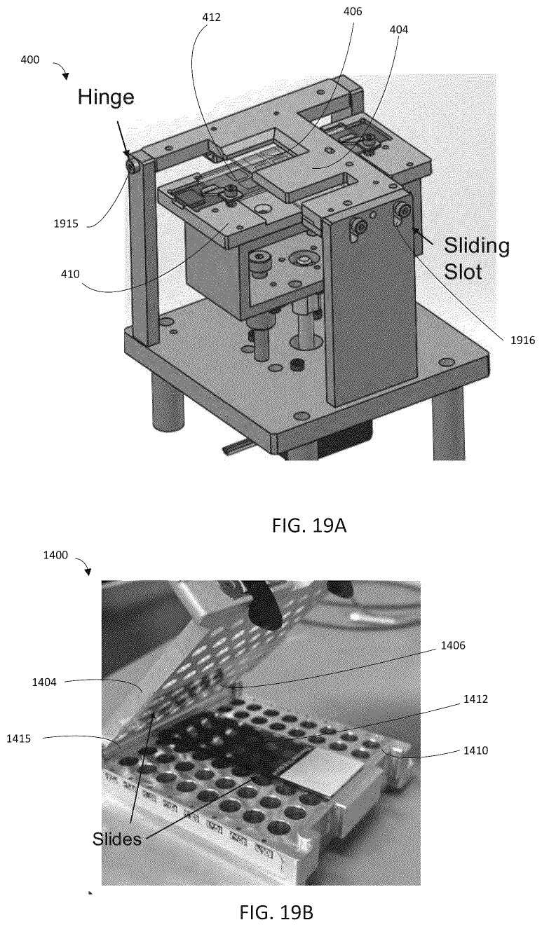

In some aspects, the angled closure of FIGS. 17-18 may be accomplished using a variety of hardware components. For example, FIG. 19A depicts the sample handling apparatus 400 including a hinge 1915 and a sliding slot 1916 coupled to the first member 404 in accordance with some example implementations. The hinge 1915 and/or the sliding slot 1916 may be configured to position the first member 404 and/or the first substrate 406 at an angle relative to the second member 410 and/or the second substrate 412. This angle can be as small as 0 degree.

FIG. 19B depicts a sample handling apparatus 1400 in an open configuration with a first substrate 1406 coupled to the first member 1404 and the second member 1410 retaining the second substrate 1412 in accordance with some example implementations. In some aspects, closing the first member 1404 over the second member 1410 via the hinge 1415 may provide the angled closure described herein in at least FIGS. 17-18 and the corresponding description.

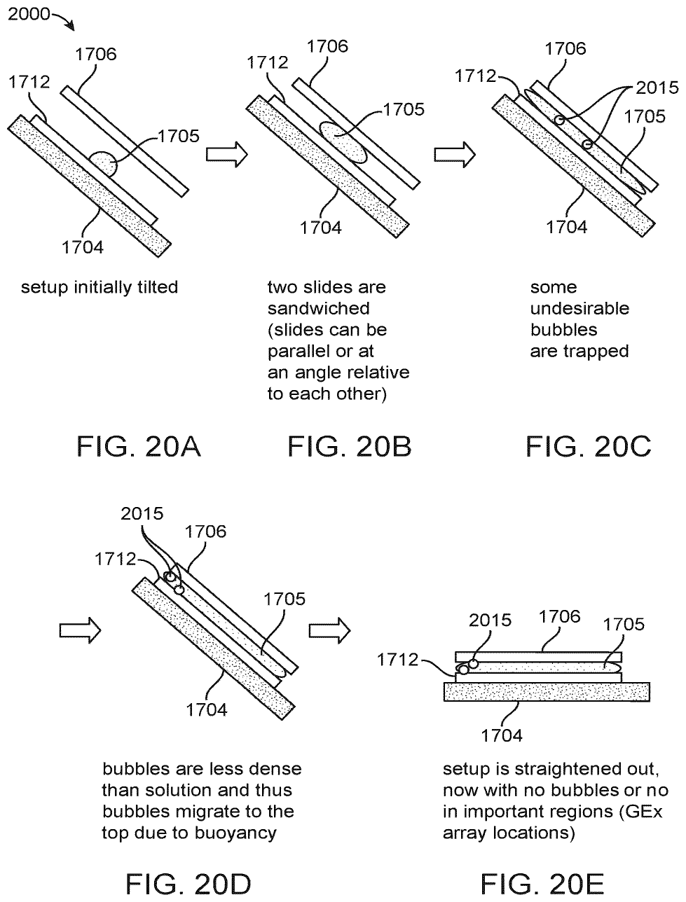

FIGS. 20A-20E show an example workflow 2000 for an angled sandwich assembly in accordance with some example implementations. As shown in FIG. 20A, the base 1704 may be positioned tilted at an angle. The slide 1712 may be disposed flat on the base 1704 and at the same angle. The angle may be determined such that a drop (e.g., drop 1705) placed on the surface of the slide 1712 will not fall off the surface (e.g., due to gravity). The angle may be determined by a gravitational force versus any surface force to move the drop away from the off the slide 1712.

FIG. 20B depicts the slide 1706 and the slide 1712 being sandwich together as the slide 1706 and the slide 1712 move toward each other and the slide 1706 contacts the drop 1705. In some aspects, the slides 1706 and 1712 may be parallel or at an angle relative to each other during the sandwiching. In some embodiments the angle of the slides may be achieved via a sample handling apparatus (e.g., the sample handling apparatus 400, the sample handling apparatus 1400, or the like).

FIG. 20C depicts one or more air bubbles 2015 trapped within the drop 1705 during the sandwiching of the slides 1706 and 1712.

As shown in FIG. 20D, the one or more air bubbles 2015 may be less dense than the liquid reagent drop 1705 and the one or more air bubbles 2015 may migrate up in a superior direction due to buoyancy. In some aspects, as the one or more air bubbles 2015 reach the top (e.g., uppermost part of the drop 1705), the bubbles may release or otherwise be removed from the drop 1705.

FIG. 20E depicts the base 1704, the slide 1706, and the slide 1712 straightened along an axis and the one or more bubbles 2015 removed from the drop 1705 or removed from a region of interest between the slides 1706 and 1712.

In some aspects, the angled closure of FIGS. 17-18 and 20 may occur in response to detecting a bubble (e.g., bubble 2015) within the drop 1705. Additionally or alternatively, the angled closures described herein may occur during each sandwiching of the slides (e.g., the slides 1706 and 1712). A sensor may be configured to detect a bubble in the liquid reagent drop 1705 responsive to a slide (e.g., the slide 1706) or a tissue sample (e.g., tissue sample 302) contacting at least a portion of the drop 1705.

In some embodiments, the drop (e.g., drop 1705) includes permeabilization reagents (e.g., any of the permeabilization reagents described herein). In some embodiments, the rate of permeabilization of the biological sample is modulated by delivering the permeabilization reagents (e.g., a fluid containing permeabilization reagents) at various temperatures.

In some embodiments, the permeabilization reagents are dried permeabilization reagents. In some embodiments, the dried permeabilization reagents are disposed on a substrate (e.g., the first substrate, the second substrate). In some embodiments, delivering the fluid (e.g., by any of the fluid delivery methods described herein) solubilizes the dried permeabilization reagents. In some embodiments, solubilizing the permeabilization reagents results in permeabilization of the biological sample. In some embodiments, delivering the fluid to solubilize dried reagents is delivered via an aperture in a gasket. In some embodiments, delivering the fluid to solubilize dried reagents is delivered through a via-hole. In some embodiments, the fluid solubilizing dried reagents includes the use of a syringe. In some embodiments, the fluid solubilizing dried reagents includes the capillary flow.

### Sample and Array Alignment Devices and Methods

Spatial analysis workflows generally involve contacting a sample with an array of features. Aligning a sample with a reagent medium (or, in some embodiments, the array) is an important step in performing spatialomic (e.g., spatial transcriptomic) assays. The ability to efficiently generate robust experimental data for a given sample can depend greatly on the alignment of the sample and the reagent medium (or the array). Traditional techniques require samples to be placed directly onto a reagent medium (or the array). For example, current methods of aligning biological samples with barcoded areas in spatial transcriptomics assays involve a user carefully placing the biological sample onto a substrate that includes a plurality of barcoded probes. This approach can require skilled personnel and additional experimental time to prepare a section of the sample and to mount the section of the sample directly on the reagent medium (or the array). Misalignment of the sample and the reagent medium (or the array) can result in wasted reagent medium (or a wasted array), extended sample preparation time, and inefficient use of samples, which may be limited in quantity.

The systems, methods, and computer readable mediums described herein can enable efficient and precise alignment of samples and arrays, thus facilitating the spatial transcriptomic imaging and analysis workflows or assays described herein. Thus, in some embodiments, an advantage of the devices described is providing an alignment tool for users to align a sample with a barcoded area. Samples, such as portions of tissue, can be placed on a first substrate. The first substrate can include a slide onto which a user can place a sample of the tissue. An array, (e.g., such as a reagent array, or such as a spatially barcoded array) can be formed on a second substrate. The second substrate can include a slide and the array can be formed on the second substrate. The use of separate substrates for the sample and the array can beneficially allow user to perform the spatialomic (e.g., spatial transcriptomic) assays described herein without requiring the sample to be placed onto an array substrate. The sample holder and methods of use described herein can improve the ease by which users provide samples for spatialomic (e.g., spatial transcriptomic) analysis. For example, the systems and methods described herein alleviate users from possessing advanced sample or tissue sectioning or mounting expertise. Additional benefits of utilizing separate substrates for samples and arrays can include improved sample preparation and sample imaging times, greater ability to perform region of interest (ROI) selection, and more efficient use of samples and array substrates. The devices of the disclosure can reduce user error during the assay analysis, thereby also reducing sample analysis costs. In some embodiments, another advantage of the devices of the disclosure is a reduction in the number of aberrations or imaging imperfections that may arise due to user error in aligning a biological sample with a barcoded area of the substrate. In some embodiments, the devices of the disclosure allow for pre-screening of samples for areas of interest. In some embodiments, the devices of the disclosure allow for archived samples to be examined.

The sample substrate and the array substrate, and thus, the sample and the array, can be aligned using the instrument and processes described herein. The alignment techniques and methods described herein can generate more accurate spatialomic (e.g., spatial transcriptomic) assay results due to the improved alignment of samples with an array (e.g., such as a reagent array, or such as a spatially barcoded array).

In some embodiments, a workflow described herein comprises contacting a sample disposed on an area of a first substrate with at least one feature array of a second substrate. In some embodiments, the contacting comprises bringing the two substrates into proximity such that the sample on the first substrate may be aligned with the barcoded array on the second substrate. In some instances, the contacting is achieved by arranging the first substrate and the second substrate in a sandwich assembly. In some embodiments, the workflow comprises a prior step of mounting the sample onto the first substrate.

Alignment of the sample on the first substrate with the array on the second substrate may be achieved manually or automatically (e.g., via a motorized alignment). In some aspects, manual alignment may be done with minimal optical or mechanical assistance and may result in limited precision when aligning a desired region of interest for the sample and the barcoded array. Additionally, adjustments to alignment done manually may be time-consuming due to the relatively small time requirements during the permeabilization step.

It may be desirable to perform real-time alignment of a tissue slide (e.g., the pathology slide 303) with an array slide (e.g., the slide 304 with barcoded capture probes 306). In some implementations, such real-time alignment may be achieved via motorized stages and actuators of a sample handling apparatus (e.g., the sample handling apparatus 400, the sample handling apparatus 1400, or the like).

An exemplary spatial analysis workflow disclosed herein is provided. In some instances, the methods include providing a first substrate that includes a biological sample and a second substrate that includes a plurality of capture probes. In some instances, the plurality of capture probes include oligonucleotide probes. In some instances, the plurality of capture probes includes analyte capture agents that can detect an analyte of interest (e.g., a protein) as described herein. In some instances, the plurality of capture probes includes analyte capture agents that can detect an analyte of interest (e.g., a protein) as described herein. In some instances, the plurality of capture probes includes a capture domain comprising a sequence complementary to a capture handle sequence present in an analyte capture agent. The methods can be performed in an order determined by a person skilled in the art. For example, as an exemplary overview, a first substrate include a biological sample. The biological sample then is stained using any of the methods described herein. In some instances, the biological sample is imaged, capturing the stain pattern created during the stain step. In some instances, the biological sample then is destained. After destaining, in some instances, a second substrate that includes capture probes as described herein is added to the first substrate. In some instances, the biological sample is permeabilized using methods disclosed herein (e.g., a solution that includes proteinase K and SDS). Permeabilization releases the analytes from the biological sample. Analytes then migrate from the first substrate and are captured by the second substrate. In some instances, after capture, the analytes and/or the probe can be amplified and the sequence can be determined using methods disclosed herein.

In some instances, the first substrate and the second substrate are arranged in a sandwich assembly, e.g., as described herein. It is noted that the terms first substrate and second substrate do not necessarily connote the particular order or location of the biological sample or capture probes. For example, in one instance, the first substrate includes the biological sample and the second substrate includes capture probes. In another instance, the first substrate includes capture probes and the second substrate includes the biological sample. In some embodiments, the tissue permeabilization process begins when the sample is contacted with the permeabilization buffer. During the permeabilization process, analytes are released from the sample. In some embodiments, analytes that are released from the permeabilized sample diffuse to the surface of the second substrate and are captured on the feature array (e.g., on barcoded probes). In some instances, there is a gap between the first and the second substrate. In some instances, the gap is about 1, 2, 2.5, 3, 4, 5, 6, 7, 8, 9, 10, 11, 12, 12.5, 13, 14, 15, 16, 17, 18, 19, 20, 21, 22, 23, 24, 25 μm or more. In some embodiments, second substrate is placed in direct contact with the sample on the first substrate ensuring no diffusive spatial resolution losses. In some embodiments, an alignment mechanism is configured to maintain a separation between the first and second substrates when the first and second substrates are aligned. In some embodiments, the alignment mechanism is configured to maintain the separation such that at least a portion of the sample on the first substrate contacts at least a portion of the reagent medium on the second substrate. In some embodiments, the separation between the first and second substrates is between 2 microns and 1 mm, measured in a direction orthogonal to a surface of the first substrate that supports the sample. In some instances, the first substrate and the second substrate are separated (e.g., pulled apart). In some embodiments, the sample analysis (e.g., cDNA synthesis) can be performed on the first substrate after the first substrate and the second substrate are separated. In some embodiments, the substrate comprising the biological sample can be discarded or archived after the first substrate and the second substrate are separated.

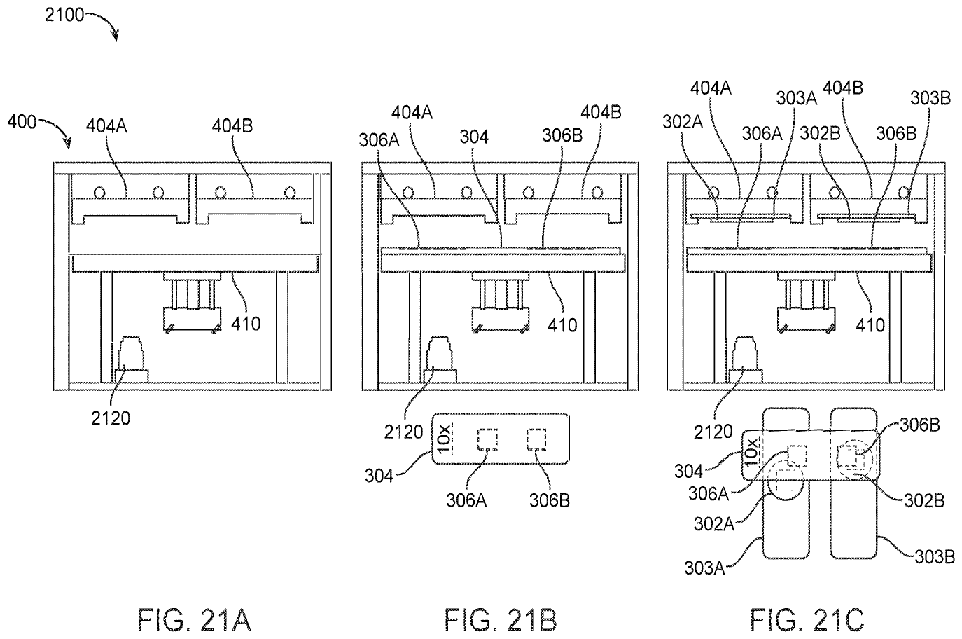

FIGS. 21A-21C depict a workflow 2100 for loading slides into a sample handling apparatus for later alignment in accordance with some example implementations.

FIG. 21A depicts the example sample handling apparatus 400 with no slides loaded into the apparatus 400. As shown, the sample handling apparatus 400 includes two first members 404, the second member 410, and an image capture device 2120. While two first members 404 and a single second member 410 are shown in the FIGS. 21A-21C, it will be appreciated that more or fewer first members 404 and/or second members 410 are possible. While the image capture device 2120 is shown in a position inferior to the second member 410, other locations for the image capture device 2120 are possible and more or fewer image capture devices 2120 are also possible.

FIG. 21B depicts the sample handling apparatus 400 with a gene expression slide (e.g., slide 304 with barcoded capture probes 306) loaded into the second member 410. A bottom portion of the FIG. 21B shows a top view of the slide 304. As shown, the slide 304 includes two regions with barcoded capture probes 306A and 306B, respectively.

FIG. 21C depicts the sample handling apparatus 400 with a pathology slide 303A and a pathology slide 303B loaded into first members 404A and 404B, respectively. As shown, the pathology slides 303A and 303B include tissue samples 302A and 302B, respectively. A bottom portion of FIG. 21C shows a top view of an initial alignment of the gene expression slide 304 with the pathology slides 303A and 303B after loading.

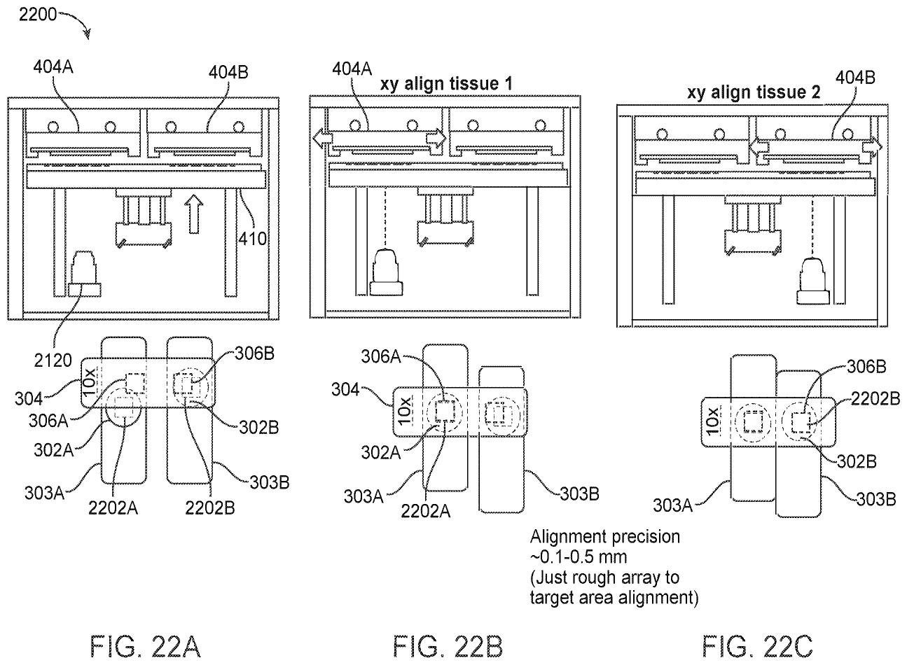

FIGS. 22A-22C depict a workflow 2200 for aligning the loaded slides of the sample handling apparatus 400. FIGS. 22A-22C are similar to and adapted from FIGS. 21A-21C and the workflow 2200 may occur after the workflow 2100.

FIG. 22A shows the sample handling apparatus 400 of FIG. 21C with the second member 410 moved up towards the first members 404A and 404B. In some aspects, bringing the second member 410 closer to the first members 404 may make alignment of the desired regions of the slides 303 and 304 easier to achieve. The movement of the second member 410 may be performed by an adjustment mechanism (e.g., adjustment mechanism 415) of the sample handling apparatus 400. The bottom portion of FIG. 22A shows a top view of the initial alignment of the slides 303A, 303B, and 304. As further shown, the tissue samples 302A and 302B include regions of interest 2202A and 2202B, respectively. The regions of interest 2202A and 2202B may be selected by a user prior to loading the slides 303 into the sample handling apparatus 400 or may be determined after imaging of the tissue samples 302A and 302B. In some aspects, the regions of interest 2202A and 2202B may be marked on the slides 303A and 303B, marked on an image of the tissue samples 302A and 302B, or otherwise identified by a user when aligning.

FIG. 22B depicts an alignment of the barcoded capture probe area 306A with the tissue sample region of interest 2202A. The alignment may occur in an xy plane and by moving the first member 404A in an xy direction to optically and vertically align the capture probes 306A with the region of interest 2202A. For example, as shown in the bottom portion of FIG. 22B, the top view of the slides 303A and 304 show that the capture probes 306A are aligned with the region of interest 2202A of the tissue sample 302A (e.g., dashed lines). In some aspects, the image capture device 2120 may aid in the alignment of the slides 303 and 304 by providing images of the capture probes 306A, the sample 302A, and/or the region of interest 2202A. In some aspects, the alignment precision may be within approximately 0.1-0.5 mm.

In some aspects, the movement of the first member 404A may be performed by an alignment mechanism configured to move the slide 303A (e.g., the first substrate 406, the first substrate 1406, the slide 1706, or the like) along a first plane (e.g., the xy plane of the slide 303A). In some implementations, the alignment mechanism may be configured to move the gene expression slide 304 (e.g., the second substrate 412, the second substrate 1412, the slide 1712, or the like) along a second plane (e.g., the xy plane of the slide 304).

FIG. 22C depicts an alignment of the barcoded capture probe area 306B with the tissue sample region of interest 2202B. The alignment may occur in an xy plane and by moving the first member 404B in an xy direction to optically and vertically align the capture probes 306B with the region of interest 2202B. For example, as shown in the bottom portion of FIG. 22C, the top view of the slides 303B and 304 show that the capture probes 306B are aligned with the region of interest 2202B of the tissue sample 302B. In some aspects, the image capture device 2120 may aid in the alignment of the slides 303 and 304 by providing images of the capture probes 306B, the sample 302B, and/or the region of interest 2202B.

In some aspects, the movement of the first member 404B may be performed by an alignment mechanism configured to move the slide 303B (e.g., the first substrate 406, the first substrate 1406, the slide 1706, or the like) along a first plane (e.g., the xy plane of the slide 303B). In some implementations, the alignment mechanism may be configured to move the gene expression slide 304 (e.g., the second substrate 412, the second substrate 1412, the slide 1712, or the like) along a second plane (e.g., the xy plane of the slide 304).

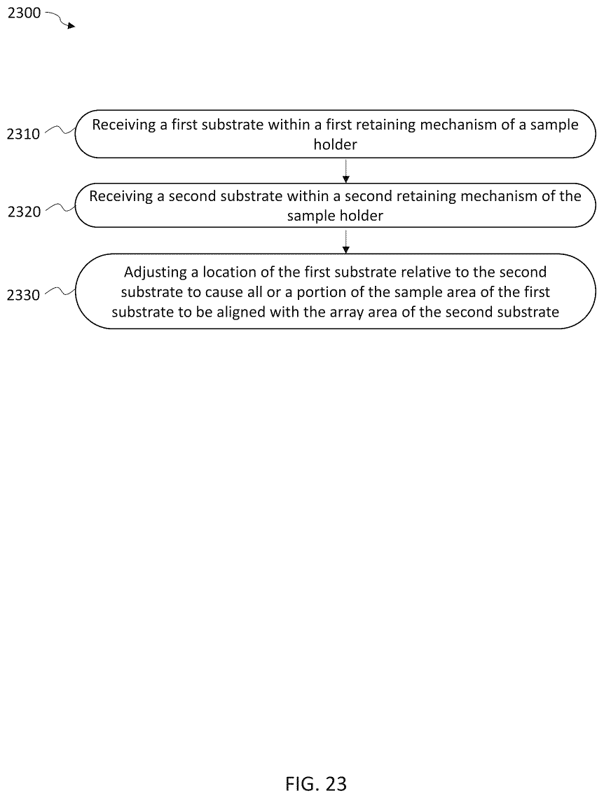

FIG. 23 is a process flow diagram illustrating an example process 2300 for aligning a sample area with an array area according to some implementations of the current subject matter. At 2310, a first substrate can be received within a first retaining mechanism of a sample handling apparatus, such as sample handling apparatuses 400, 1400, or 3500. A user can provide or position the first substrate within the first retaining mechanism of the sample handling apparatus 400. The first substrate can include a sample applied to the first substrate by a user. The first substrate can also include a sample area into which the sample is to be placed. The first substrate can further include a sample area indicator identifying the sample area. In some embodiments, the first substrate can include a fiducial mark. The first retaining mechanism can include one or more spring members configured to apply a force to the first substrate to maintain contact between the first substrate and a first member of the sample handling apparatus 400 on which the first retaining mechanism is configured.

At 2320, a second substrate can be received within a second retaining mechanism of the sample handling apparatus 400. The second substrate can include an array of reagent medium formed within an array area indicator identifying the array on the second substrate. In some embodiments, the array area indicator can be provided on the sample handling apparatus 400. A user can provide or position the second substrate within the second retaining mechanism of the sample handling apparatus 400. The second retaining mechanism can include one or more spring members configured to apply a force to the second substrate to maintain contact between the second substrate and a second member of the sample holder on which the second retaining mechanism is configured.

At 2330, a location of the first substrate can be adjusted relative to the second substrate to cause all or a portion of the sample area of the first substrate to be aligned with the array area of the second substrate. In some embodiments, adjusting the location of the first substrate relative to the second substrate can be performed to cause the sample area indicator to be aligned with the array area indicator. In some embodiments, the location of the first substrate relative to the second substrate can be adjusted by a user. For example, the user can manually manipulate the first member and/or the second member of the sample holder so as to adjust a location of the first substrate and/or the second substrate within the sample holder to cause the sample area to be aligned with the array area. In some embodiments, the location of the first substrate can be adjusted relative to the second substrate, which can be fixed in position within the sample handling apparatus 400. In some embodiments, the location of the second substrate can be adjusted relative to the first substrate, which can be fixed in position within the sample handling apparatus 400. In some embodiments, the second substrate can be fixed in place within the sample handling apparatus 400 and the first retaining mechanism can be adjusted to cause all or a portion of the sample area to be aligned with the array area.

In some embodiments, a user can adjust the location of the first substrate and/or the second substrate while viewing the first substrate and/or the second substrate within the sample handling apparatus 400. For example, the user can view the first substrate and the second substrate via a microscope of the instrument configured to provide the sample holder within a field of view of the microscope. In some embodiments, the instrument can include a display providing a view of the first substrate and the second substrate within the sample handling apparatus.

In some embodiments, adjusting the location of the first substrate relative to the second substrate can further include viewing the first substrate and the second substrate within the sample holder and adjusting the first retaining mechanism and/or the second retaining mechanism to cause all or a portion of the sample area to be aligned with the array area. In this way, the sample handling apparatus 400 can advantageously support efficient and precise alignment by providing multiple, different ways to perform the alignment. In some embodiments, the adjusting can be performed in the absence of a sample area indicator configured on the first substrate and/or in the absence of an array area indicator configured on the second substrate.

In some embodiments, the location of the first substrate and/or the second substrate can be adjusted within the sample holder by a user interacting with a physical positioning device configured on the sample handling apparatus 400, or on the instrument while viewing the first substrate and the second substrate. The physical positioning device can include a joy stick, a pointing stick, a button, or the like. In some embodiments, the instrument can be configured with computer-readable, executable instructions stored in a memory of the instrument. The instructions, when executed, can perform the adjusting automatically based on image data associated with the sample handling apparatus 400, the first substrate, and/or the second substrate. In some embodiments, the instrument can be configured with a display providing a graphical user interface (GUI). A user can interact with the GUI to adjust the location of the first substrate relative to the second substrate to cause all or a portion of the sample area indicator to be aligned with respect to the array area indicator.

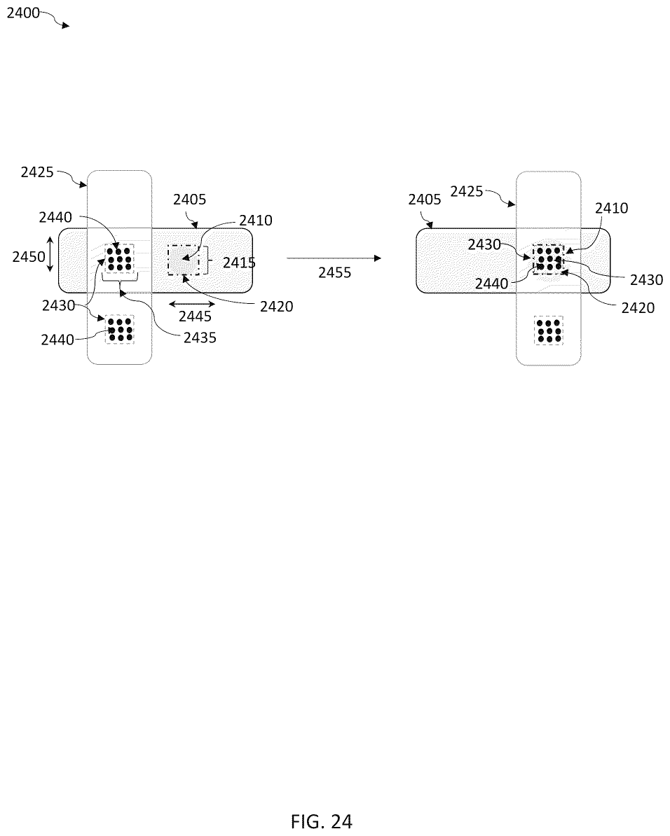

FIG. 24 is a diagram 2400 illustrating adjusting a location of the first substrate relative to the second substrate to align all or a portion of a sample area with an array area. As shown in FIG. 24, and with reference to operation 2330 described in relation to FIG. 23, a first substrate 2405 can include a sample 2410 positioned by a user within a sample area 2415 identified by a sample area indicator 2420 of the first substrate 2405. In some embodiments, the first substrate 2405 may not include the sample area indicator 2420. The second substrate 2425 can include one or more array area indicators 2430 indicating a location of an array area 2435. Each array area 2435 can include an array 2440 therein.

The sample handling apparatus 400 can be configured to enable adjustment of the first substrate 2405 and/or the second substrate 2425 along a first axis 2445 and a second axis 2450. The first axis 2445 can be considered a later axis within a transverse plane corresponding to the mounting surface in which the first substrate 2405 and the second substrate 2425 are received within the sample handling apparatus 400. The second axis 2450 can be considered a longitudinal axis within the transverse plane corresponding to the mounting surface in which the first substrate 2405 and the second substrate 2425 are received within the sample handling apparatus 400.

As shown in FIG. 24, adjusting 2455 the first substrate 2405 relative to the second substrate 2425 can be performed to cause all or a portion of the sample area 2415 to be aligned with the array area 2435. Additionally, or alternatively, the adjusting 2455 (e.g., operation 2330 of FIG. 23) can further cause the sample area indicator 2420 to be aligned with respect to the array area indicator 2430. In this way, the adjusting 2455 can cause the sample 2410 to be aligned with the array 2440.

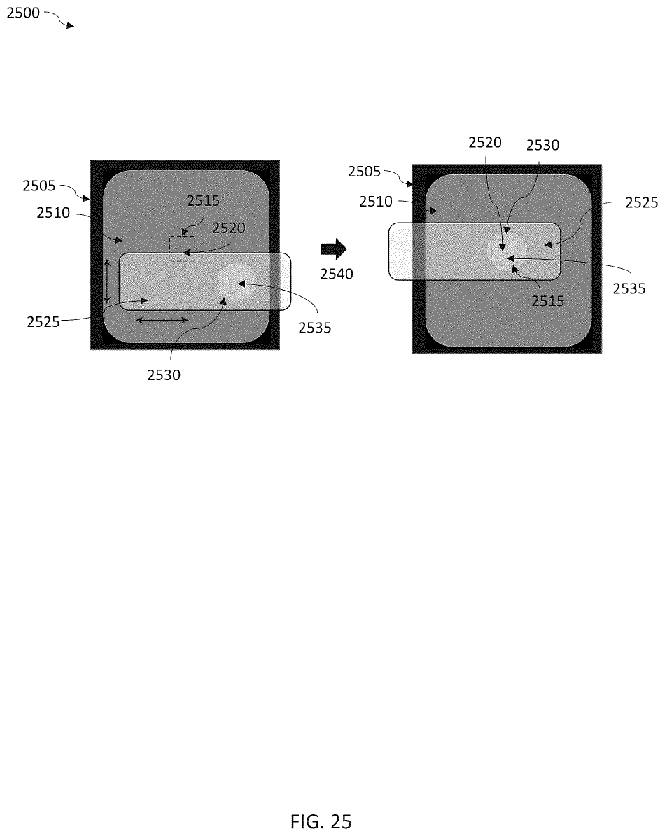

FIG. 25 is a diagram illustrating an exemplary embodiment for adjusting a location of the first substrate relative to the second substrate based on an array area indicator configured within a sample handling apparatus 400 according to some implementations of the current subject matter. As shown in FIG. 25, a sample handling apparatus 400 can include a retaining mechanism 2505 configured with a transparent surface 2510. The transparent surface 2510 can include an array area indicator 2515 identifying an array area 2520. The array area indicator 2515 can be configured on a first surface of the retaining mechanism 2505, for example a first surface corresponding to the transparent surface 2510. In some embodiments, the array area indicator 2515 can be configured on a second surface of the retaining mechanism 2505, the second surface opposite the transparent surface 2510. In some embodiments, the array area indicator 2515 can be disposed on a first retaining mechanism (e.g., the first retaining mechanism 1408) or a second retaining mechanism (e.g., the second retaining mechanism 609) of a sample holder (e.g., the sample holder 400, the sample holder 1400, or the like).

As shown in FIG. 25, a substrate 2525 including a sample 2530 positioned within a sample area 2535 can be received within the retaining mechanism 2505. Adjusting 2540 the substrate 2525 relative to the transparent surface 2510 can be performed to cause all or a portion of the sample area 2535 to be aligned with the array area 2520.

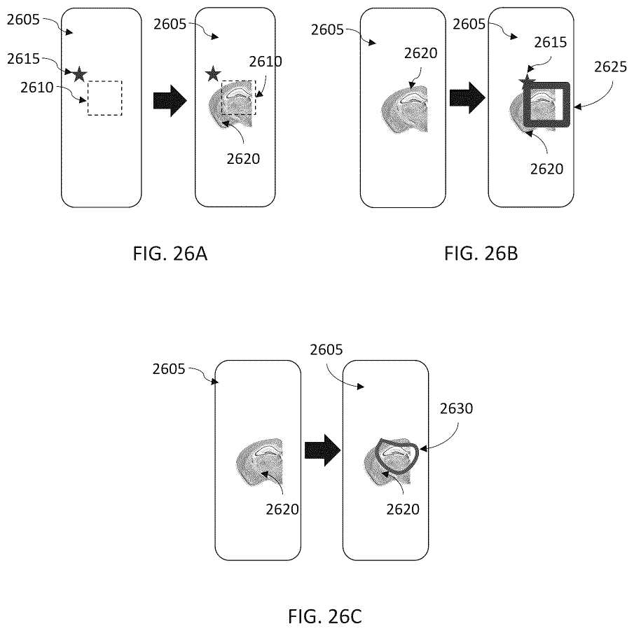

FIGS. 26A-26C are diagrams illustrating exemplary embodiments for indicating a sample area of a substrate according to some implementations of the current subject matter. The substrate described in relation to FIGS. 26A-26C can be equivalent to the first substrate described in relation to FIGS. 23 and 24. To indicate a sample area of a substrate on to which a sample is placed a variety of embodiments can be considered.

As shown in FIG. 26A, a substrate 2605 can include a sample area indicator 2610.

The sample area indicator 2610 can be provided by the manufacturer of the substrate such that the sample area indicator is provided on the substrate 2605 prior to a user placing a sample 2620 onto the substrate 2605. In some embodiments, the sample area indicator 2610 can be applied to a first side of the substrate 2605 prior to applying the sample 2620 to the first side of the substrate 2605. In some embodiments, the sample area indicator 2610 can be applied to a second side of the substrate 2605. The second side of the substrate 2605 can be opposite the first side of the substrate 2605. In some embodiments, the sample area indicator 2610 can be applied to the second side of the substrate 2605 after the sample 2620 has been applied to the first side of the substrate 2605.

As further shown in FIG. 26A, the substrate 2605 can include a fiducial mark 2615. The fiducial mark 2615 can be applied to the first side of the substrate 2605 or to the second side of the substrate 2605. The fiducial mark 2615 can be used to aid alignment of the sample area on a first substrate 2605 with an array area on second substrate, such as second substrate 2425 described in relation to FIG. 24. The fiducial mark 2615 can include a variety of non-limiting shapes and formats, such as variously shaped applied or embedded markings or etchings, suitable to provide a fiducial reference on the substrate 2605.

As shown in FIG. 26B, the sample area indicator can include a stamp or a sticker 2625. The stamp or sticker 2625 can be applied to the second side of the substrate 2605 after the sample 2620 has been applied to the first side of the substrate 2605 by a user.

As shown in FIG. 26C, the sample area indicator can be applied as a drawing 2630 on the second side of the substrate 2605 after the sample 2620 has been applied to the first side of the substrate 2605 by a user. In some embodiments, the drawing 2630 can be drawn by a user with a marker suitable for marking the substrate 2605.

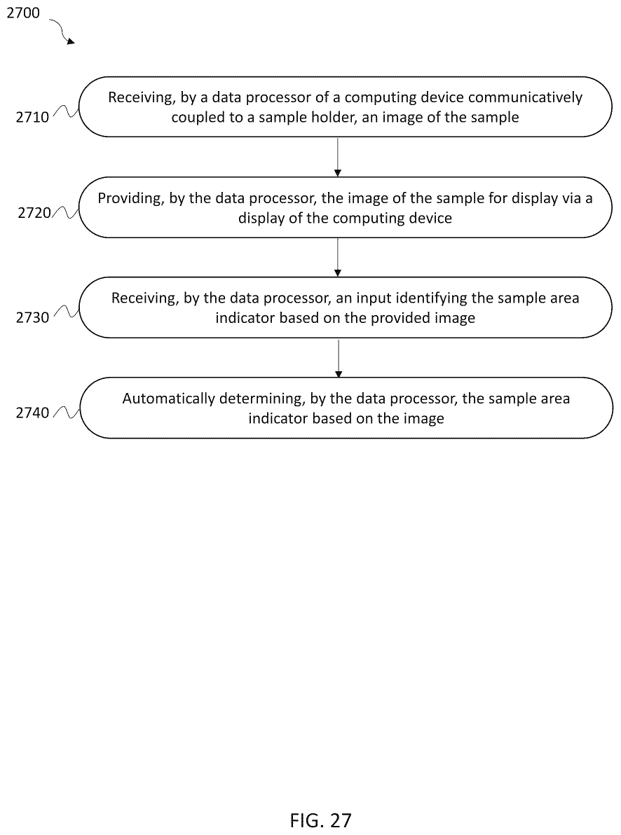

FIG. 27 is a process flow diagram illustrating an example process 2700 for automatically determining a sample area indicator based on a received image of the sample according to some implementations of the current subject matter. The system, methods, and mediums described herein can be configured to determine a sample area indicator based on an image of a sample. At 2710, an image of a sample can be received by a data processor of a computing device communicatively coupled to a sample handling apparatus 400. The sample handling apparatus 400 can receive and retain a substrate including the sample therein. The computing device can be further communicatively coupled to an image capture device 2120, such as a microscope, a camera, an optical sensor, an imaging device, or the like configured to acquire and provide an image of the sample to the computing device. In some embodiments, the computing device can be communicatively coupled to a focus motor associated with the image capture device 2120. In some embodiments, the data processor of the computing device can be configured to receive the image of the sample from a data processor of a remote computing device communicatively coupled to the computing device at which the process 2700 is performed.

At 2720, the data processor can provide the image of the sample for display via a display of the computing device. In some embodiments, the image of the sample can be provided for display via a GUI configured within the display of the computing device.

At 2730, the data processor can receive an input identifying the sample area indicator based on the provided image. For example, the display of the computing device can include a touch-screen display configured to receive a user input identifying the sample area indicator on the displayed image. In some embodiments, the GUI can be configured to receive a user provided input identifying the sample area indicator.

At 2740, the data processor can automatically determine the sample area indicator based on the image. The data processor can be configured to access and execute computer-readable, executable instructions configured to automatically determine the sample area indicator based on a variety of features included in the image. For example, the data processor can automatically determine the sample area indicator based on an outline of the tissue present within the image. This approach can be used when the sample area is smaller than the array area. In some embodiments, the data processor can automatically determine the sample area indicator based on a stamp or a sticker that is visible in the image and was applied to the first substrate by a user. In some embodiments, the data processor can automatically determine the sample area indicator based on a fiducial mark located on the first substrate that is visible in the image. In some embodiments, the data processor can automatically determine the sample area indicator based on a drawing that is visible in the image and was applied to the first substrate by a user.

In some embodiments, the data processor can access and execute computer-readable, executable instructions configured to automatically determine the sample area indicator based on sample area indicator data which can be stored in a memory of the computing device. In some embodiments, the sample area indicator data can be imported into the computing device from a second computing device that is remote from and communicatively coupled to the computing device automatically determining the sample area indicator associated with the sample in the image.

In some embodiments, the data processor can access and execute computer-readable, executable instructions configured to automatically determine the sample area indicator based on processing the sample image using image segmentation functionality. In some embodiments, the data processor can access and execute computer-readable, executable instructions configured to automatically determine the sample area indicator based on a type of sample, a size of sample, a shape of the sample, and/or an area of the sample.

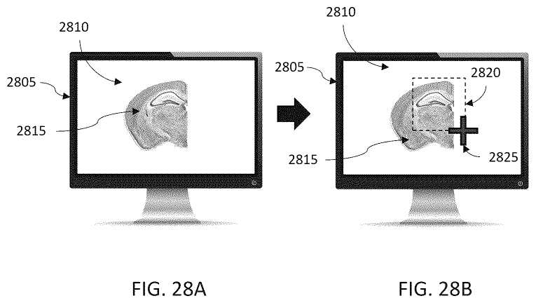

FIGS. 28A-28B are diagrams illustrating an exemplary embodiment for receiving an input identifying a sample area indicator based on an image of a sample as described in relation to operation 2730 of FIG. 27. As shown in FIG. 28A, a computing device 2805 can include a display 2810. The display 2810 can be configured to provide an image 2815 of a sample. As shown in FIG. 28B, a user may interact with the display 2810 to provide an input identifying the sample area indicator 2820. For example, the user can manipulate a mouse or other input device in relation to the image 2815 of the sample so as to provide an input identifying the sample area indicator 2820. The user input can be provided to select all or a portion of the image 2815 to be associated with the sample area indicator 2820. The selection can be provided by the user dragging a cursor 2825 over the image 2815 to form the sample area indicator 2820. In some embodiments, the input can be provided by a user cropping the image 2815 such that the perimeter of the cropped image forms the sample area indicator 2820.

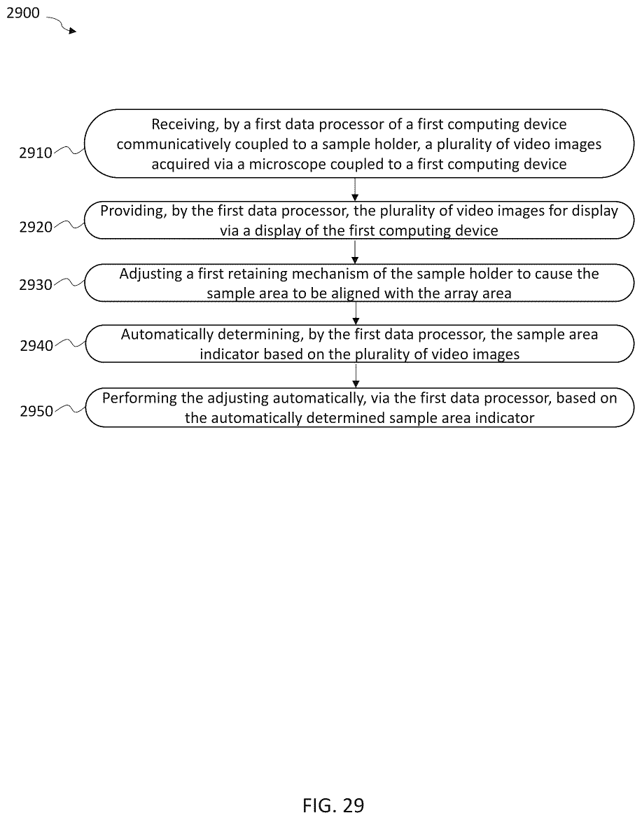

FIG. 29 is a process flow diagram illustrating an example process 2900 for automatically determining a sample area indicator based on a plurality of received video images according to some implementations of the current subject matter. At 2910, a data processor of a computing device communicatively coupled to a sample handling apparatus 400 can receive a plurality of video images. The plurality of video images can be acquired by and received from via an image capture device 2120, such as a microscope, a camera, an optical sensor, an imaging device, or the like, communicatively coupled to the data processor. The plurality of video images can include the sample positioned on a first substrate and the array located on the second substrate. The plurality of video images can include the second substrate overlaid atop the first substrate. In some embodiments, the data processor of the computing device can be configured to receive the image of the sample from a data processor of a remote computing device communicatively coupled to the computing device at which the process 2900 is performed.

At 2920, the data processor can provide the plurality of video images for display via a display of the computing device. In some embodiments, the plurality of video images can be provided for display via a GUI configured within the display of the computing device. In some embodiments, the plurality of video images can be provided to a data processor of a second computing device. The second computing device can be remote from the first computing device and can be communicatively coupled to the first computing device at which the plurality of video images were first received. The second computing device can be configured to provide the plurality of video images for display via a display of the second computing device. In some embodiments, the second computing device can be configured to receive an input from a user identifying a sample area indicator associated with the sample positioned on the first substrate. The user can provide the input identifying the sample area indicator to the second computing device as previously described above.

At 2930, a user can manually adjust a first retaining mechanism of the sample handling apparatus 400 to cause the sample area of the first substrate to be aligned with the array area of the second substrate. In some embodiments, the user can adjust the first retaining mechanism of the sample handling apparatus 400 to cause the sample area of the first substrate to be aligned with an array area configured within the sample handling apparatus 400. The user can adjust the first retaining mechanism based on viewing the plurality of video images provided by the first computing device or the second computing device.

At 2940, in addition, or in alternative, to the manual adjustment performed at 2930, the data processor of the first computing device can automatically determine the sample area indicator based on the plurality of video images. The data processor of the first computing device can be configured to access and execute computer-readable, executable instructions configured to automatically determine the sample area indicator based on a variety of features included in the plurality of video images. For example, the data processor can automatically determine the sample area indicator based on an outline of the tissue present within the plurality of video images. This approach can be used when the sample area is smaller than the array area. In some embodiments, the data processor can automatically determine the sample area indicator based on a stamp or a sticker that is visible in the plurality of video images and was applied to the first substrate by a user. In some embodiments, the data processor can automatically determine the sample area indicator based on a fiducial mark located on the first substrate that is visible in the plurality of video images. In some embodiments, the data processor can automatically determine the sample area indicator based on a drawing that is visible in the plurality of video images and was applied to the first substrate by a user.

In some embodiments, the data processor can access and execute computer-readable, executable instructions configured to automatically determine the sample area indicator based on sample area indicator data which can be stored in a memory of the computing device. In some embodiments, the sample area indicator data can be imported into the computing device from a second computing device that is remote from and communicatively coupled to the computing device automatically determining the sample area indicator associated with the sample in the plurality of video images.

At 2950, the data processor of the first computing device can perform the adjusting automatically based on the automatically determined sample area indicator. The computing device can be configured to automatically adjust the location of the first substrate relative to the second substrate to cause all or a portion of the sample area to be aligned with an array area of the second substrate via a controller that can be communicatively coupled to the sample handling apparatus 400 and to the first computing device. The controller can receive input signals from the data processor and can generate control signals causing the first retaining mechanism or the second retaining mechanism to translate within the sample handling apparatus 400 and there by adjust the location of the first substrate or the second substrate, respectively.

In some embodiments, the data processor of a second computing device, communicatively coupled to the data processor of the first computing device, can similarly be coupled to the controller and to the sample handling apparatus 400. The data processor of the second computing device can generate input signals to the controller and can cause the controller to generate control signals causing first retaining mechanism or the second retaining mechanism to translate within the sample handling apparatus 400. In this way, the location of the first substrate and/or the second substrate can be controlled and adjusted such that the sample area of the first substrate can be aligned with the array area of the second substrate.

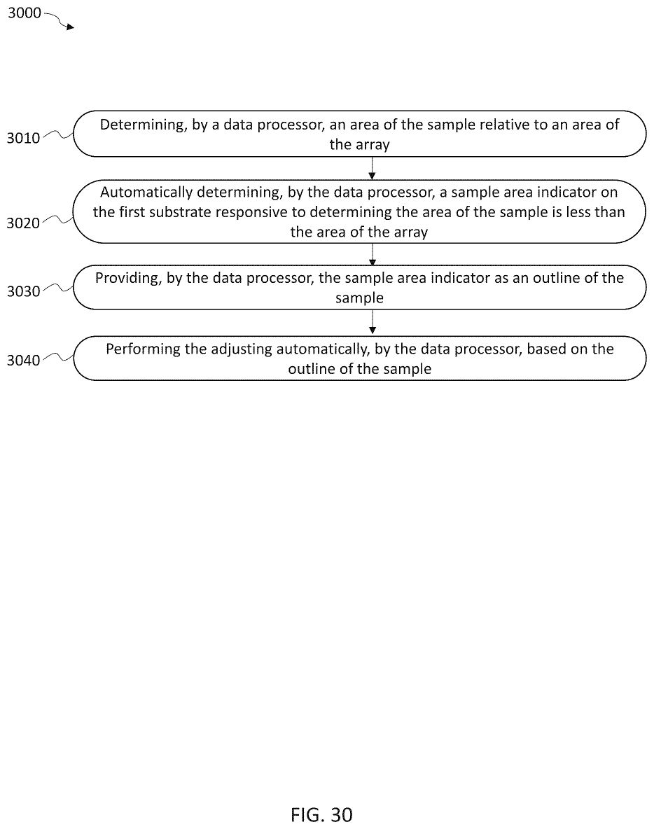

FIG. 30 is a process flow diagram illustrating an example process 3000 for automatically determining a sample area indicator responsive to determining an area of the sample according to some implementations of the current subject matter. At 3010, a data processor can determine an area of the sample relative to an area of the array. For example, during alignment of the outline of the tissue sample to the array area. When outline is not clear or tissue is larger, the slide could be annotated by indicating the target area on the tissue with a marker, sticker, etc. In some aspects, the alignment may utilize image processing in the instrument. For example, the tissue slide may be scanned first, then the outline of the tissue may be determined using image processing. If the tissue is larger than the array, the target area may be annotated. In this case, the annotation may include an annotation that the instrument can detect through image processing. The annotation may include a special marker blocking a specific wavelength or allowing a specific wavelength through.

At 3020, the data processor can automatically determine a sample area indicator on the first substrate responsive to determining the area of the sample is less than the area of the array. For example, after the tissue slide is scanned and the outline of the tissue is determined using image processing, the outline may be compared to the area of the array to determine the area of the sample is less than the area of the array.

At 3030, the data processor can provide the sample area indicator as an outline of the sample. For example, the sample area indicator can be provided in a display of the computing device.

At 3040, the data processor can perform the adjusting automatically based on the outline of the sample. As described above, the data processor of the computing device can be configured to automatically adjust the location of the first substrate relative to the second substrate to cause all or a portion of the sample area to be aligned with an array area of the second substrate via a controller that can be communicatively coupled to the sample handling apparatus 400 and to the computing device. For example, the data processor may be configured to fit the outline of the sample within the array area. The alignment of the outline may be to the array itself, a virtual outline on a UI, or some alignment reference marks elsewhere in the instrument (sample handling apparatus 400) that may indicate the array position. The controller can receive input signals from the data processor and can generate control signals causing the first retaining mechanism or the second retaining mechanism to translate within the sample handling apparatus 400 and thereby adjust the location of the first substrate or the second substrate, respectively to fit the outline of the sample within the array area.

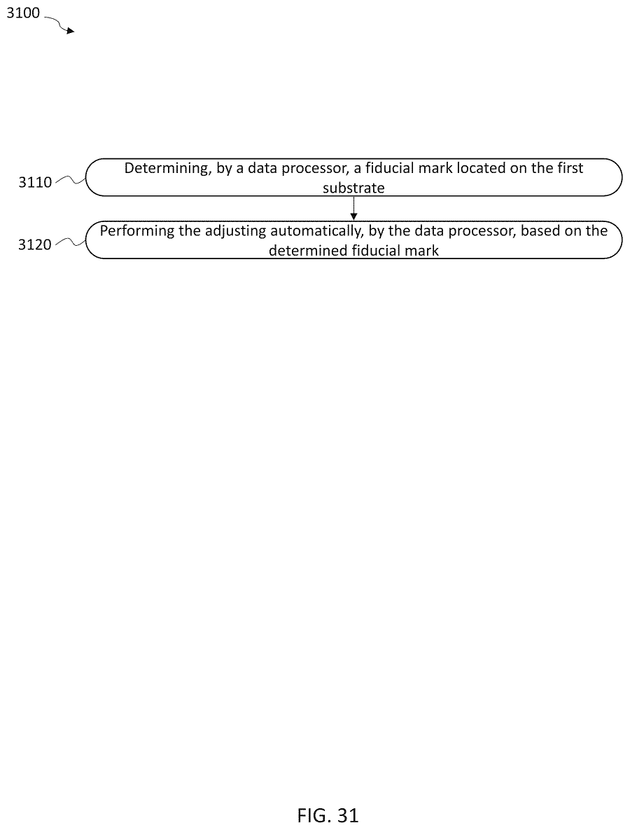

FIG. 31 is a process flow diagram illustrating an example process 3100 for determining a fiducial mark located on a first substrate according to some implementations of the current subject matter. At 3110, a data processor can determine a fiducial mark located on the first substrate. For example, the data processor may utilize computer vision or image processing techniques to identify the fiducial. The fiducial may include a high contrast or uniquely shaped mark to aid in determination of the fiducial via image processing or other methods.

At 3120, the data processor can perform the adjusting automatically based on the determined fiducial mark. As described above, the data processor of the computing device can be configured to automatically adjust the location of the first substrate relative to the second substrate to cause all or a portion of the sample area to be aligned with an array area of the second substrate via a controller that can be communicatively coupled to the sample handling apparatus 400 and to the computing device. In some aspects, the adjusting may be based on the location of the determined fiducial. For example, the fiducial may provide a reference point for aligning the first substrate with the second substrate. The controller can receive input signals from the data processor and can generate control signals causing the first retaining mechanism or the second retaining mechanism to translate within the sample handling apparatus 400 and there by adjust the location of the first substrate or the second substrate, respectively.

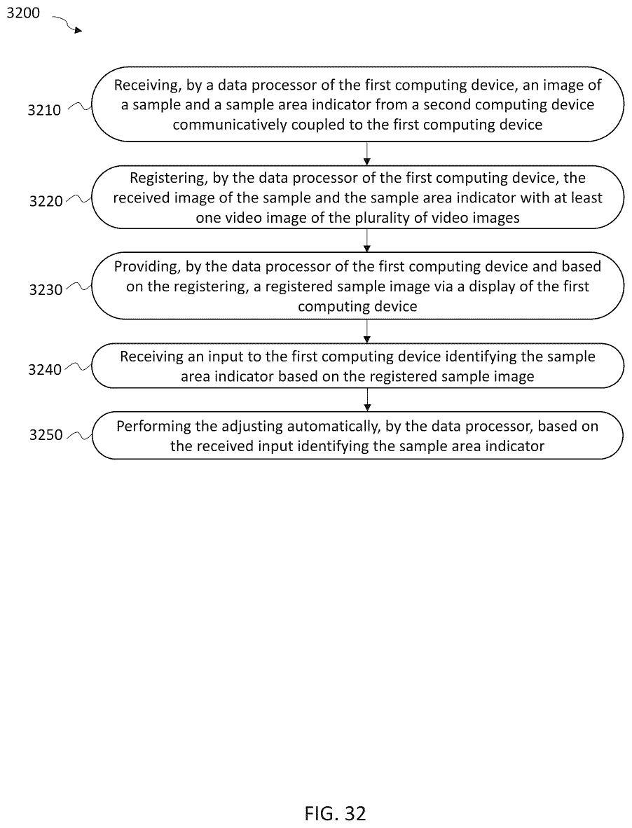

FIG. 32 is a process flow diagram illustrating an example process 3200 for identifying a sample area indicator based on a registered sample image according to some implementations of the current subject matter. At 3210, a data processor of a first computing device can receive an image of a sample and a sample area indicator from a second computing device communicatively coupled to the first computing device.

At 3220, the data processor of the first computing device can register the received image of the sample and the sample area indicator with at least a video image of a plurality of video images. The plurality of video images can be acquired via an image capture device 2120, such as a microscope, a camera, an optical sensor, an imaging device, or the like, communicatively coupled to the data processor of the first computing device.

At 3230, the data processor of the first computing device can provide, based on the image registration, a registered sample image via a display of the first computing device. For example, the registered sample image can be provided in a display of the first computing device.

At 3240, an input identifying the sample area indicator in the registered sample image can be received at the first computing device. For example, a user can provide an input to a GUI provided in a display of the first computing device. In some embodiments, the display can receive the input directly from the user or via an input device, such as a mouse or a stylus, coupled to the display.

At 3250, the data processor can perform the adjusting automatically based on the received input identifying the sample area indicator. The computing device can be configured to automatically adjust the location of the first substrate relative to the second substrate to cause all or a portion of the sample area to be aligned with an array area of the second substrate via a controller that can be communicatively coupled to the sample handling apparatus 400 and to the first computing device. The controller can receive input signals from the data processor and can generate control signals causing the first retaining mechanism or the second retaining mechanism to translate within the sample handling apparatus 400 and there by adjust the location of the first substrate or the second substrate, respectively.

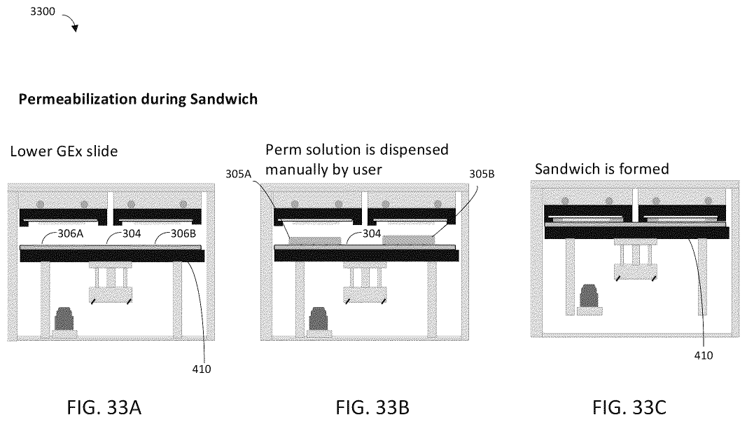

FIGS. 33A-33C depict a workflow 3300 for permeabilization of a sample (e.g., sample 302) of the sample handling apparatus 400. FIGS. 33A-33C are similar to and adapted from FIGS. 22A-22C and the workflow 3300 may occur after the workflow 2200. In some embodiments, the workflow 3300 can occur after one or more of process 2300 described in relation to FIG. 23, process 2700 described in relation to FIG. 27, process 2900 described in relation to FIG. 29, process 3000 described in relation to FIG. 30, process 3100 described in relation to FIG. 31, and process 3200 described in relation to FIG. 32.

After alignment of the slides 303 and 304 (e.g., as shown in FIG. 22C), a permeabilization solution (e.g., permeabilization solution 305) may be added. The permeabilization solution 305 may create a permeabilization buffer in the sandwich (e.g., within the gap 307) which permeabilizes or digests the tissue sample (e.g., sample 302). The analytes and/or mRNA transcripts of the tissue sample 302 may release, diffuse across the gap 307 toward the capture probes 306, and bind on the capture probes 306 (e.g., as shown in FIG. 3).

As shown in FIG. 33A, after alignment of the slides 303 and 304, the second member 410 may be lowered to facilitate in adding the permeabilization solution 305.

FIG. 33B depicts the permeabilization solution 305 dispensed on the slide 304. As shown, the permeabilization solution is dispensed in two volumes 305A and 305B located in proximity to the capture probes 306A and 306B, respectively. In some aspects, the permeabilization solution 305 may be dispensed manually by a user or automatically via a component of the sample handling apparatus 400.

FIG. 33C depicts a sandwich formed by the slide 303, the slide 304, and the sample 302. During the sandwiching of the slides and sample, the permeabilization solution 305 may begin to digest the sample 302 and release analytes and or mRNA transcripts of the sample 302 for capture by the capture probes 306A and 306B. In some aspects, the sandwich may be formed by moving the second member 410 up towards the first members 404A and 404B such that the sample 302 contacts at least a portion of the permeabilization solution 305 and the slides 303 and 304 are within a threshold distance along an axis orthogonal to the slides (e.g., along a z axis). The movement of the second member 410 may be performed by an adjustment mechanism (e.g., the adjustment mechanism 415) of the sample handling apparatus 400.

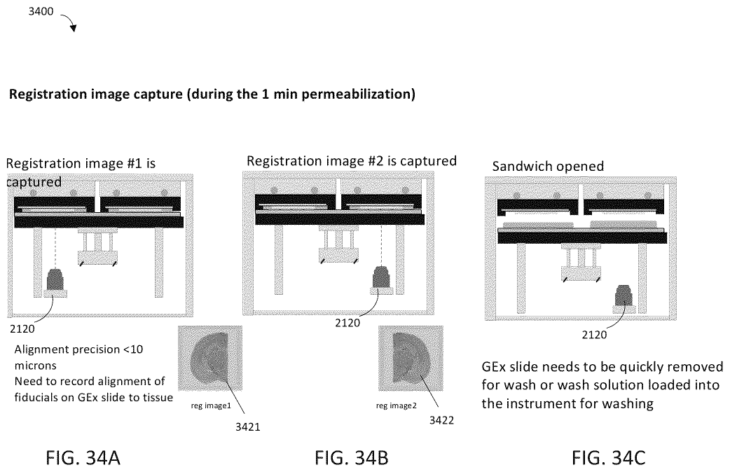

FIGS. 34A-34C depict a workflow 3400 for image capture of the sandwiched slides of the sample handling apparatus 400 during a permeabilization step in accordance with some example implementations. FIGS. 34A-34C are similar to and adapted from FIGS. 33A-33C and the workflow 3400 may occur after the workflow 3300. In some embodiments, the workflow 3400 can occur after one or more of process 2300 described in relation to FIG. 23, process 2700 described in relation to FIG. 27, process 2900 described in relation to FIG. 29, process 3000 described in relation to FIG. 30, process 3100 described in relation to FIG. 31, and process 3200 described in relation to FIG. 32.

After adding the permeabilization solution (e.g., permeabilization solution 305) to the aligned slides, it may be beneficial to capture images of the aligned tissue sample 302 and/or the barcoded capture probes 306 to aid in mapping gene expressions to locations of the tissue sample 302. As such, the image capture device 2120 may be configured to capture images of the aligned tissue sample 302, regions of interest 2202, and/or the barcoded capture probes 306 during a permeabilization step.

FIG. 34A depicts the image capture device 2120 capturing a registration image of the aligned region of interest 2202A and the capture probes 306A (e.g., alignment shown in FIGS. 22B-32) during permeabilization. The bottom portion of FIG. 34A shows an example registration image 3421 captured by the image capture device 2120 of the tissue sample 302A. As further shown, it may be desirable that an alignment precision of the slides 303 and 304 be less than 10 microns. The registration image 3421 may record alignment of any fiducial's on the gene expression slide 304 with respect to the tissue 302.

FIG. 34B depicts the image capture device 2120 capturing a second registration image of the aligned region of interest 2202B with the capture probes 306B (e.g., alignment shown in FIGS. 22C-32) during permeabilization. The bottom portion of FIG. 34B shows an example second registration image 3422 captured by the image capture device 2120 of the tissue sample 302B.

In some aspects, the permeabilization step may occur within one minute and it may be beneficial for the image capture device 2120 to move quickly between the different sandwiched slides and regions of interest. Although a single image capture device 2120 is shown, more than one image capture device 2120 may be implemented.

FIG. 34C depicts the sample handling apparatus 400 after any registration images (e.g., registration images 3421 and/or 3422) are captured and the permeabilization step may be completed. As shown, the sandwich may be opened and any of the slides 303 and 304 may be removed for washing or a wash solution may be loaded into the instrument for washing. For example, the gene expression slide 303 may be removed for washing, library prep, gene sequencing, image registration, gene expression mapping, or the like.

In some aspects, the sandwich may be opened by moving the second member 410 away from the first members 404, or vice versa. The opening may be performed by the adjustment mechanism 415 of the sample handling apparatus 400.

While workflows 2100, 2200, 3300, and 3400 are shown and described with respect to the sample handling apparatus 400, the workflows 2100, 2200, 3300, and 3400 may also be performed with respect to the sample handling apparatus 1400, the sample handling apparatus 3500, or another sample handling apparatus in accordance with the implementations described herein. In some embodiments, the processes 2300, 2700, 2900, 3000, 3100, and 3200 may also be performed with respect to the sample handling apparatus 1400, the sample handling apparatus 3500, or another sample handling apparatus in accordance with the implementations described herein.

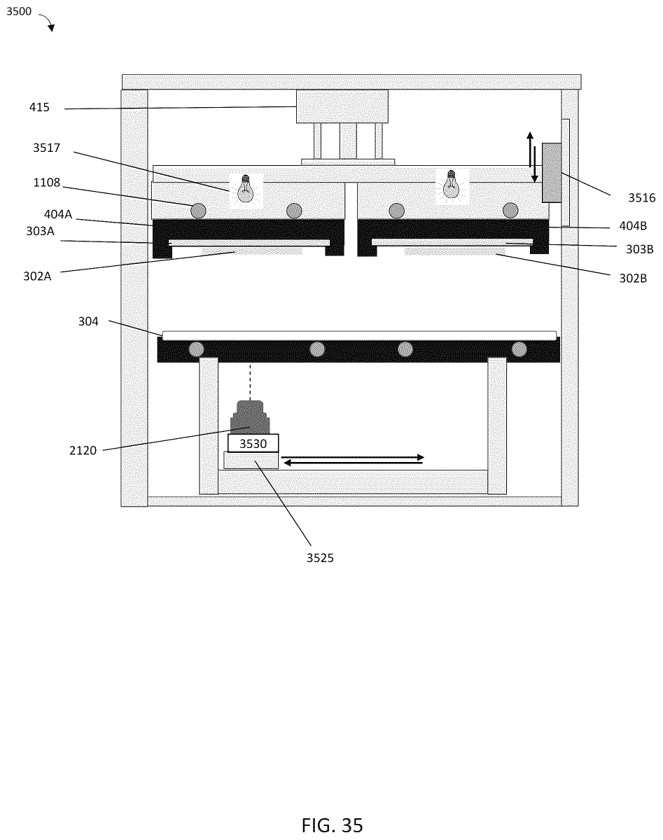

FIG. 35 is a diagram of an example sample handling apparatus 3500 in accordance with some example implementations. The sample handling apparatus 3500 is similar to and adapted from the sample handling apparatus 400 of FIGS. 4-13C.

As shown, the sample handling apparatus 3500 includes an adjustment mechanism 415, a linear guide 3516, a trans-illumination source 3517, one or more heaters 1108, first members 404A and 404B, tissue slides 303A and 303B, tissue samples 302A and 302B, a gene expression slide 304, and the image capture device 2120. In the example of FIG. 35, the adjustment mechanism 415 is configured to move one or more first members 404 along an axis orthogonal to the first members 404 (e.g., along a z axis). The linear guide 3516 may aid in the movement of the one or more first members 404 along the axis. As further shown, the image capture device 2120 may be mounted on a shuttle 3525 configured to move the image capture device 2120 laterally from a position inferior to the first member 404A to a position inferior to the first member 404B. The shuttle 3525 may allow the image capture device 2120 to capture images of the tissues 302A and 302B aligned with portions of the gene expression slide 304. The trans-illumination source 3517 may facilitate image capture of the aligned portions by providing sufficient illumination of the image capture area. In some embodiments, the image capture device 2120 can be coupled to a focus motor 3530. The focus motor 3530 can include one or actuators configured to adjust one or more aspects of focusing the image capture device 2120. For example, the focus motor 3530 can be configured to control a focal point, a focus, or a zoom setting of the image capture device 2120. In some embodiments, the focus motor 3530 can be manually controlled by a user. In some embodiments, the focus motor 3530 can be automatically controlled by a camera control, such as camera control 4610, configured in the sample handling apparatus described herein.

### Exemplary Fluid Delivery Schemes

In the example sandwich maker workflows described herein, a liquid reagent (e.g., the permeabilization solution 305) may fill a gap (e.g., the gap 307) between a tissue slide (e.g., slide 303) and a capture slide (e.g., slide 304 with barcoded capture probes 306) to warrant or enable transfer of target molecules with spatial information. Described herein are examples of filling methods that may suppress bubble formation and suppress undesirable flow of transcripts and/or target molecules or analytes. Robust fluidics in the sandwich making described herein may preserve spatial information by reducing or preventing deflection of molecules as they move from the tissue slide to the capture slide.

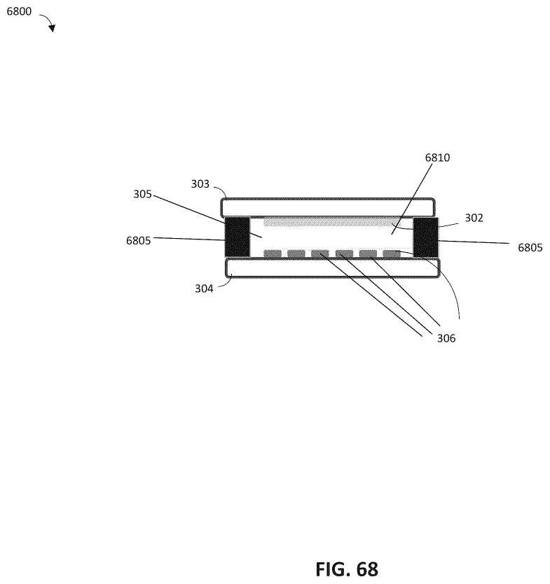

FIG. 68 shows an exemplary sandwich configuration 6800 where a first substrate (e.g., pathology slide 303), including a biological sample 302 (e.g., a tissue section), and a second substrate (e.g., slide 304 including spatially barcoded capture probes 306) are brought into proximity with one another in accordance with some example implementations. As shown in FIG. 68, a liquid reagent drop (e.g., permeabilization solution 305) is introduced on the second substrate in proximity to the capture probes 306 and in between the biological sample 302 and the second substrate (e.g., slide 304). The permeabilization solution 305 may release analytes that can be captured by the capture probes 306 of the array. As further shown, one or more spacers 6805 may be positioned between the first substrate (e.g., pathology slide 303) and the second substrate (e.g., slide 304). In some embodiments, the one or more spacers 6805 may can include one or more spacing members that assist in maintaining the spacing and/or approximately parallel arrangement of the first and second substrates. Spacing members can be connected to either or both of the first and the second substrates. In some aspects, the terms “spacer” and “gasket” are used herein to describe spacing members that may assist in maintaining the spacing and/or approximately parallel arrangement of the first and second substrates and the terms may be used interchangeably.

The one or more spacers 6805 may be configured to maintain a separation distance between the first substrate and the second substrate. The one or more spacers 6805 can be placed on the first substrate adjacent to the biological sample 302 and in between the first substrate and the second substrate. The one or more spacers 6805 can be placed on the second substrate adjacent to the array 306 and in between the first substrate and the second substrate. By doing so, the one or more spacers 6805 can create a chamber (e.g., chamber 6810) in which solutions (e.g., a buffer, a permeabilization solution 305) are contained throughout the permeabilization and analyte migration process. In some embodiments, more than one spacer is used. In some embodiments, the one or more spacers 6805 have a height of about 2 μm, about 12.5 μm, about 15 μm, about 17.5 μm, about 20 μm, about 22.5 μm, or about 25 μm. In some embodiments, the height of each spacer has a height of about 50 μm, about 100 μm, about 200 μm, about 300 μm, about 400 μm, about 500 μm, about 600 μm, about 700 μm, about 800 μm, about 900 μm, or about 1000 μm. The one or more spacers 6805 may be formed of a material having uniform thickness or of a material having a variable (e.g., beveled) thickness.

In some embodiments, the one or more spacers 6805 may create a fully or partially enclosed chamber around the biological sample (e.g., tissue sample 302 or a region of interest) and/or the array 306. The fully enclosed one or more spacers 6805 can be configured to any shape. In some embodiments, the fully enclosed (e.g., encompassed) chamber created by the one or more spacers 6805 is one of a square or a rectangle. In some embodiments, one or more spacers 6805 conform to the shape of the biological sample 302. For example, the one or more spacers 6805 are shown herein to have an example shape, an example height, and maintain an example separation distance (e.g., 12.5 μm), although other values and shapes are possible and may depend on the liquid reagent, the biological sample 302, the capture probes 306, or the like.

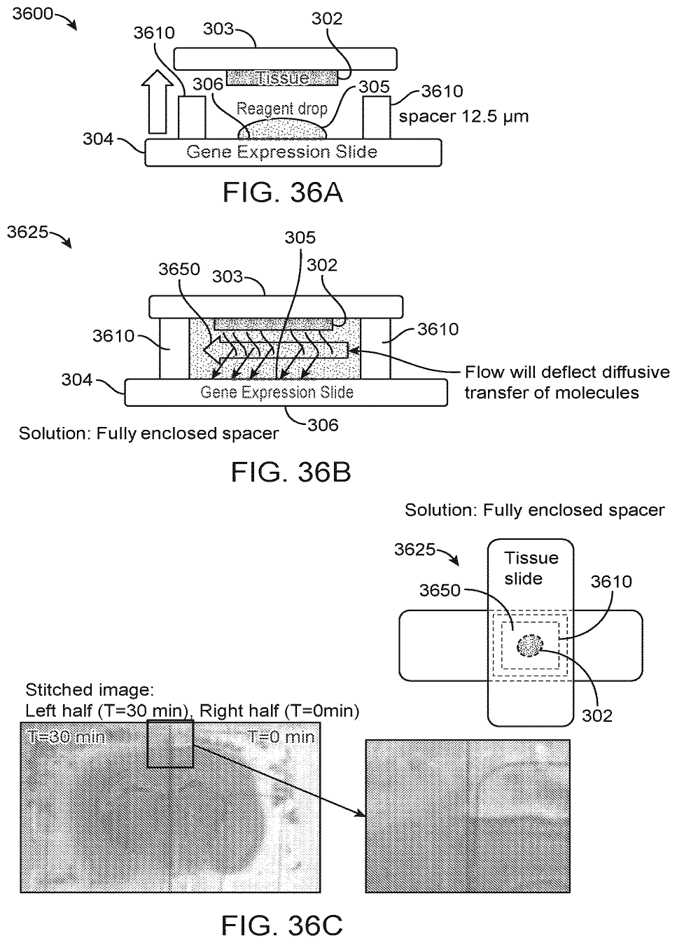

FIG. 68 shows an example of a fully formed sandwich creating a chamber 6810 formed from the one or more spacers 6805, the first substrate (e.g., the pathology slide 303), and the second substrate (e.g., the slide 304) in accordance with some example implementations. In the example of FIG. 68, the liquid reagent (e.g., the permeabilization solution 305) fills the volume of the chamber 6810 and may create a permeabilization buffer that allows mRNA transcripts and/or molecules to diffuse from the biological sample 302 toward the capture probes 306 of the slide 304. In some aspects, any flow of the permeabilization buffer may deflect transcripts and/or molecules from the biological sample 302 and may affect diffusive transfer of analytes for spatial analysis. A partially or fully sealed chamber 6810 resulting from the one or more spacers 6805, the first substrate, and the second substrate may reduce or prevent flow from undesirable convective movement of transcripts and/or molecules over the diffusive transfer from the biological sample 302 to the capture probes 306. FIG. 36A shows an exemplary sandwich configuration 3600 where a first substrate (e.g., pathology slide 303), including a biological sample 302 (e.g., a tissue section), and a second substrate (e.g., slide 304 including spatially barcoded capture probes 306) are brought into proximity with one another. As shown in FIG. 36A a liquid reagent drop (e.g., permeabilization solution 305) is introduced on the second substrate in proximity to the capture probes 306 and in between the biological sample 302 and the second substrate (e.g., slide 304). The permeabilization solution 305 may release analytes that can be captured by the capture probes 306 of the array. As further shown, one or more spacers 3610 may be positioned between the first substrate (e.g., pathology slide 303) and the second substrate (e.g., slide 304). The one or more spacers 3610 may be configured to maintain a separation distance between the first substrate and the second substrate. For example, the one or more spacers 3610 are shown to have a height and maintain a separation distance of 12.5 μm, although other values are possible and may depend on the liquid reagent, the biological sample 302, the capture probes 306, or the like.

FIG. 36B shows a fully formed sandwich creating a chamber 3650 formed from the one or more spacers 3610, the first substrate (e.g., the pathology slide 303), and the second substrate (e.g., the slide 304) in accordance with some example implementations. In the example of FIG. 36B, the liquid reagent (e.g., the permeabilization solution 305) fills the volume of the chamber 3650 and may create a permeabilization buffer that allows mRNA transcripts and/or molecules to diffuse from the biological sample 302 toward the capture probes 306 of the slide 304. In some aspects, any flow of the permeabilization buffer may deflect transcripts and/or molecules from the biological sample 302 and may affect diffusive transfer of analytes for spatial analysis. A partially or fully sealed chamber 3650 resulting from the one or more spacers 3610, the first substrate, and the second substrate may reduce or prevent flow from undesirable convective movement of transcripts and/or molecules over the diffusive transfer from the biological sample 302 to the capture probes 306.

FIG. 36C depicts a top view of the configuration 3625 of FIG. 36B. As shown, the one or more spacers 3610 may fully enclose and surround the biological sample 302 and form the chamber 3650 when sandwiched between the first substrate and the second substrate. The right hand side of FIG. 36C depicts an example of reduced convection during by capturing images of the sample 302 at the start of the sandwich and at the end of the sandwich. Half of such images may be stitched together to pronounce the dominant diffusion and suppressed convection during sandwiching.

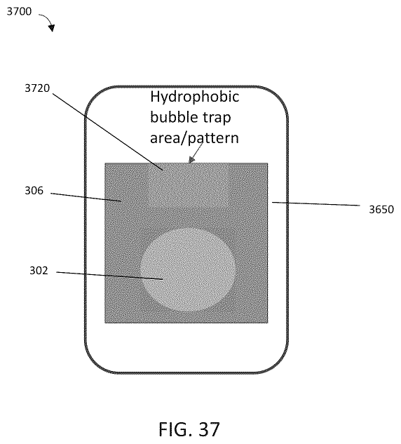

FIG. 37 depicts an example configuration 3700 for venting or removing bubbles from the chamber 3650 in accordance with some example implementations. FIG. 37 depicts a top view of the chamber 3650 where the square portion includes the capture probes 306, the circular portion includes the biological sample 302, and the rectangular portion includes a hydrophobic area 3720. The hydrophobic area 3720 may include a hydrophobic pattern that does not wet and is disposed in a portion of the chamber 3650 that is located away from an area of interest (e.g., an area where the biological sample 302 and the capture probes 306 overlap). The hydrophobic area 3720 may be configured to remove bubbles (e.g., bubbles 2015) from the chamber 3650 during the permeabilization step.

In some aspects, any combination of bubble venting or bubble removing features may be applied to the chamber, the first substrate, and/or the second substrate. For example, air permeable spacers (e.g., spacers 3610) may be configured to vent out trapped bubbles. Further, bubble venting holes disposed on the first substrate, the second substrate, and/or a spacer may be placed at strategic locations to vent bubbles. In some aspects, a sonication or vibration device may be configured to generate vibration on the first substrate and/or the second substrate during closing of the sandwich to reduce the chance of a bubble sticking to a surface of the first substrate or the second substrate. Additionally, it may be possible to increase a humidity of the chamber during sandwich closing to facilitate the filling process of the permeabilization solution or liquid reagent. Further, it may be possible to generate a vacuum in the chamber during closing to reduce or eliminate the chance of bubble trapping.

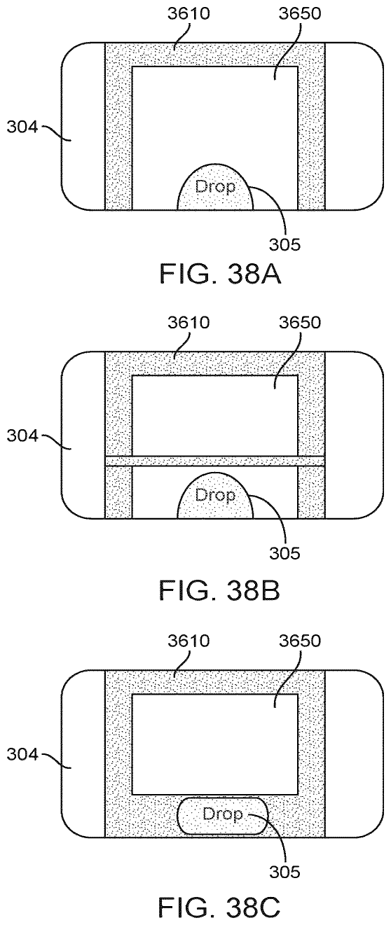

FIGS. 38A-38C show example configurations for that one or more spacers 3610 disposed on the first substrate (e.g., the pathology slide 303) and/or the second substrate (e.g., the slide 304) in accordance with some example implementations. While the slide 304 (e.g., the second substrate) is shown in FIGS. 38A-38C, the example spacer configurations may apply equally to the first substrate (e.g., the pathology slide 303) in accordance with example embodiments. In some aspects, the example spacer configurations of FIGS. 38A-38C may be combined with an angled closure workflow as described herein (e.g., workflow 1700 of FIGS. 17A-18B).

FIG. 38A is a top view of an example chamber 3650 having a partial enclosure with three sides of the one or more spacers 3610 closed. As shown, a drop of the permeabilization solution 305 is disposed along the open side of the chamber 3650 and on a surface of the slide 304. In some aspects, the angled closure of the first substrate (e.g., the pathology slide 303) contacting the drop 305 may urge the permeabilization solution toward the one or more spacers 3610 partially surrounding the drop 305. In some implementations, the three sides of the one or more spacers 3610 may at least partially surround capture probes 306 of the second substrate (e.g., slide 304) and/or the biological sample 302 of the first substrate (e.g., pathology slide 303).

FIG. 38B depicts a top view of another example chamber 3650 having a full enclosure. As shown, the one or more spacers 3610 fully surround and enclose the chamber 3650. As further shown, the drop of the permeabilization solution 305 is positioned outside of the chamber 3650 on a surface of the slide 304. As described above, an angled closure workflow (e.g., workflow 1700) of the first substrate (e.g., the pathology slide 303) over the second substrate (e.g., slide 304) may result in a dropped side of the first substrate contacting the drop 305 and urging the permeabilization solution 305 toward and within the chamber 3650. In the example of FIG. 38B, the one or more spacers 3610 may at least partially surround capture probes 306 of the second substrate (e.g., slide 304) and/or the biological sample 302 of the first substrate (e.g., pathology slide 303).

FIG. 38C depicts a top view of another example chamber 3650 having a full enclosure. As shown, the one or more spacers 3610 fully surround and enclose the chamber 3650. As further shown, the drop of the permeabilization solution 305 is positioned outside of the chamber 3650 and on a surface of the one or more spacers 3610. As described above, an angled closure workflow (e.g., workflow 1700) of the first substrate (e.g., the pathology slide 303) over the second substrate (e.g., slide 304) may result in a dropped side of the first substrate contacting the drop 305 and urging the permeabilization solution 305 toward and within the chamber 3650. In the example of FIG. 38C, the one or more spacers 3610 may at least partially surround capture probes 306 of the second substrate (e.g., slide 304) and/or the biological sample 302 of the first substrate (e.g., pathology slide 303).

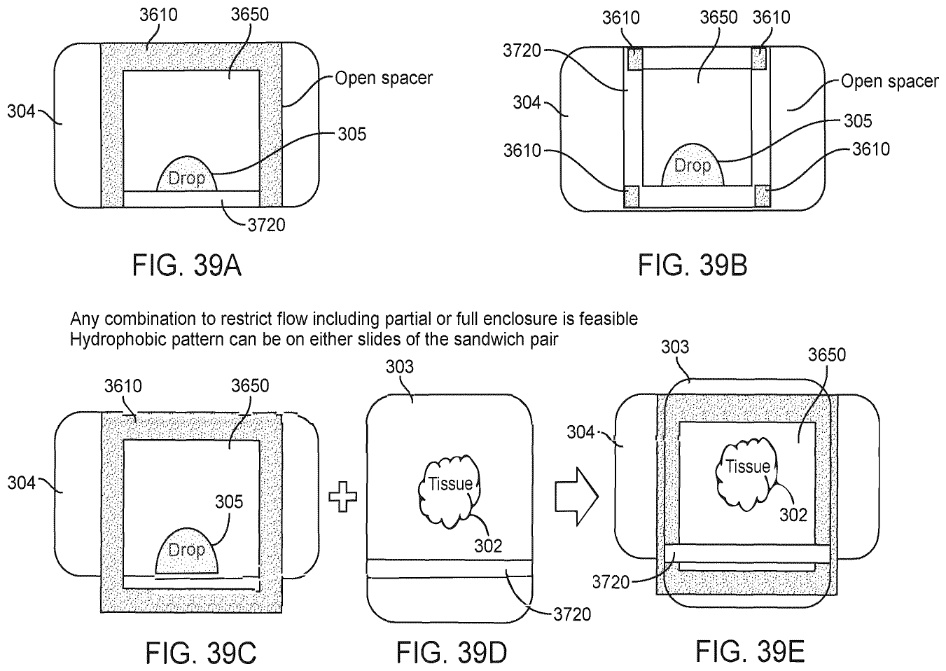

FIGS. 39A-39E depict example configurations of the one or more spacers 3610 combined with one or more hydrophobic areas 3720 in accordance with some example implementations. Any or all of the example configurations shown may be combined with an angled closure workflow (e.g., workflow 1700) for sandwiching the first substrate and the second substrate and for forming the chamber 3650.

FIG. 39A depicts a top view of an example chamber 3650. As shown, the chamber 3650 comprises three sides of the one or more spacers 3610 a fourth side comprising the hydrophobic area 3720. As further shown, the drop of the permeabilization solution 305 is located on the slide 304 proximate to the hydrophobic area 3720. As described above, an angled closure workflow (e.g., workflow 1700) of the first substrate (e.g., the pathology slide 303) over the second substrate (e.g., slide 304) may result in a dropped side of the first substrate contacting the drop 305 and urging the permeabilization solution 305 toward the opposite side and within the chamber 3650 having the three sides of the one or more spacers 3610.

FIG. 39B depicts a top view of another example chamber 3650. As shown, the chamber 3650 includes four spacers 3610 placed at the four corners of the chamber 3650 and the hydrophobic area 3720 comprising the sides of the chamber 3650. In the example of FIG. 39B, the spacers 3610 placed at the corners of the chamber 3650 may retain a minimum spacing between a first substrate (e.g., the pathology slide 303) and the second substrate (e.g., the slide 304) during sandwiching. The hydrophobic area 3720 of FIG. 39B may retain the permeabilization solution 305 within the chamber 3650 during the permeabilization step. During sandwiching, the permeabilization solution 305 may fill the volume of the chamber 3650.

In some aspects, any combination of the one or more spacers 3610, the hydrophobic area 3720, or the like may be implemented to achieve flow and/or bubble suppression. In some embodiments, the one or more spacers 3610 and/or the hydrophobic area 3720 may be disposed on either the first substrate (e.g., the pathology slide 303) or the second substrate (e.g., the slide 304).

FIG. 39C depicts a top view of an example configuration for the one or more spacers 3610 on the second substrate (e.g., the slide 304). As shown, the one or more spacers 3610 surround the drop of permeabilization solution 305 on three sides of the chamber 3650.

FIG. 39D depicts a top view of the first substrate (e.g., the pathology slide 303) including the biological sample 302 and the hydrophobic area 3720.

FIG. 39E depicts atop view of the first substrate (e.g., the pathology slide 303 of FIG. 39D) sandwiched with the second substrate (e.g., the slide 304 of FIG. 39C). As shown, the combination of the one or more spacers 3610 of FIG. 39C and the hydrophobic area 3720 of FIG. 39D form the fully enclosed chamber 3650 of FIG. 39E.

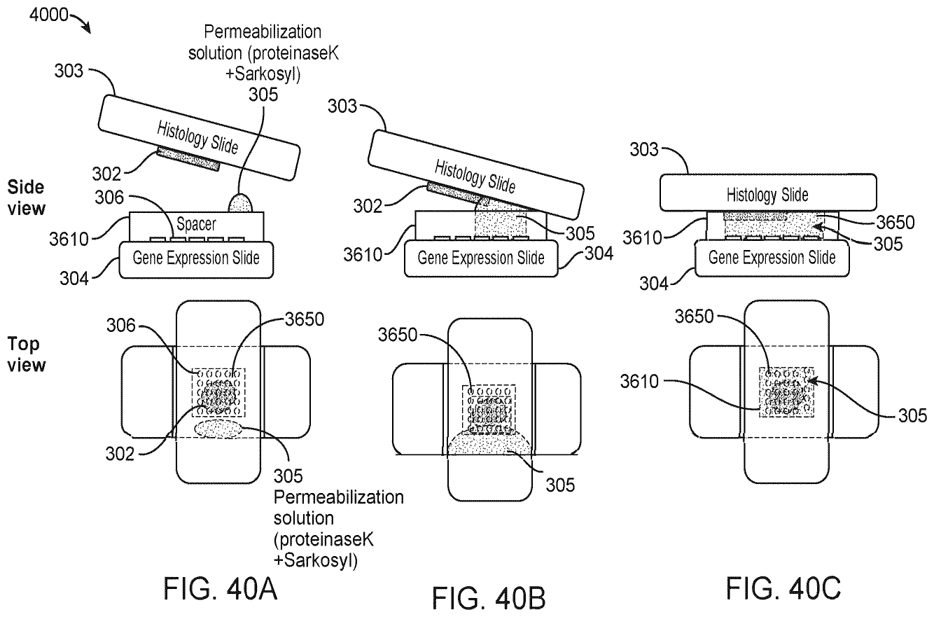

FIGS. 40A-40C depict a side view and a top view of an angled closure workflow 4000 for sandwiching a first substrate (e.g., pathology slide 303) having a tissue sample 302 and a second substrate (e.g., slide 304 having capture probes 306) in accordance with some example implementations.

FIG. 40A depicts the first substrate (e.g., the pathology slide 303 including sample 302) angled over (superior to) the second substrate (e.g., slide 304). As shown, a drop of the permeabilization solution 305 is located on top of the spacer 3610 toward the right-hand side of the side view in FIG. 40A.

FIG. 40B shows that as the first substrate lowers, or as the second substrate rises, the dropped side of the first substrate (e.g., a side of the slide 303 angled inferior to the opposite side) may contact the drop of the permeabilization solution 305. The dropped side of the first substrate may urge the permeabilization solution 305 toward the opposite direction. For example, in the side view of FIG. 40B the permeabilization solution 305 may be urged from right to left as the sandwich is formed.

FIG. 40C depicts a full closure of the sandwich between the first substrate and the second substrate with the spacer 3610 contacting both the first substrate and the second substrate and maintaining a separation distance between the two. As shown in the top view of FIGS. 40B-40C, the spacer 3610 fully encloses and surrounds the tissue sample 302 and the capture probes 306, and the spacer 3610 forms the sides of chamber 2650 which holds a volume of the permeabilization solution 305.

In some aspects, the alignment of the tissue sample 302 with the capture probes 306 shown in FIGS. 40A-40C may be performed by an alignment mechanism of a sample handling apparatus (e.g., sample handling apparatus 400, sample handling apparatus 1400, sample handling apparatus 3500, or the like).

Also provided herein are methods for delivering a fluid to a biological sample disposed on an area of a first substrate and an array disposed on a second substrate, including, delivering a fluid to the area on the first substrate, where a virtual gasket surrounds the area on the first substrate and contains the fluid within the area and assembling the second substrate with the first substrate, thereby delivering the fluid to the array and the biological sample.

Also provided herein are methods for delivering a fluid to a biological sample disposed on an area of a first substrate and an array disposed on a second substrate, including, delivering a fluid to the area on the second substrate, where a virtual gasket surrounds the area on the second substrate and contains the fluid on the array and assembling the first substrate with the second substrate, thereby delivering the fluid to the array and the biological sample.

In some embodiments, the biological sample is disposed on a first substrate. In some embodiments an array (e.g., a substrate including capture probes) is on a second substrate. In some embodiments, the first substrate including the biological sample and the second substrate including the array (e.g., a spatial array) are brought in proximity to one another such that the first substrate and the second substrate are disposed proximally to each other.

As used herein, a “partially sealed chamber” is a chamber between a first substrate and a second substrate, where a gasket is disposed between the first substrate and the second substrate.

In some embodiments of any of the methods for delivery a fluid described herein, the first substrate, the second substrate, or both, can be any of the substrates described herein. In some embodiments, the first substrate is a glass surface. In some embodiments, the second substrate is a glass surface. In some embodiments, the first substrate and the second substrate are both glass surfaces. In some embodiments, the glass surface is a glass slide. In some embodiments, the first substrate, the second substrate, or both are glass slides.

In some embodiments, the first substrate and the second substrate can be axially aligned. For example, the second substrate can be placed on top of the first substrate, or vice versa, in substantially the same orientation as the first substrate. In some embodiments, the first substrate and the second substrate are aligned in a cross-configuration. For example, the second substrate can be placed on top of the first substrate, or vice versa, at approximately a 90° angle to the first substrate. In some embodiments, the second substrate can be placed on top of the first substrate, or vice versa, at about a 40°, about 45°, about 50°, about 55°, about 60°, about 65°, about 70°, about 75°, about 80°, or about 850 angle relative to the first substrate.

In some embodiments, a gasket is disposed on the first substrate prior to aligning the first substrate and second substrate (e.g., axially, cross-configuration). In some embodiments, the gasket surrounds (e.g., encompasses) the biological sample. In some embodiments, a gasket is disposed on the second substrate prior to aligning the first substrate and the second substrate. In some embodiments, the gasket surrounds (e.g., encompasses) the array on the substrate. In some embodiments, the gasket has no apertures (e.g., an opening). In some embodiments, the gasket includes one or more apertures and a hydrophobic coating is disposed at one or more apertures in the gasket to help prevent overflow after delivery of the fluid (e.g., a permeabilization solution).

In some embodiments, the gasket can be made of rubber, silicone, or a similar material to create a seal with the first substrate. In some embodiments, the gasket can be made of a material that is hydrophobic. Accordingly, different fluids, including a permeabilization solution, can be delivered to the various apertures of the gasket. In some embodiments, the engagement of the bottom of the gasket and top of the gasket in contact with the first substrate and the second substrate creates ample pressure to maintain a partially sealed chamber where fluid (e.g., a buffer, a permeabilization solution) is delivered. In some embodiments, the gasket is a virtual gasket.

As used herein, a “virtual gasket” is a hydrophobic coating that functions similar to a physical gasket such that fluid delivered within the virtual gasket is contained within the perimeter of the virtual gasket. In some embodiments, the hydrophobic coating helps localize the fluid (e.g., permeabilization solution) over the biological sample, including a region of interest. In some embodiments, the hydrophobic coating controls the volume between the first substrate and the second substrate after alignment (e.g., axially, cross-configuration) assembly. In some embodiments, the hydrophobic coating is applied with a stamp. For example, the hydrophobic coating can be applied with a stamp to the first substrate, the second substrate, or both. In some embodiments, the virtual gasket is drawn. In some embodiments, the virtual gasket is drawn with a wax or a paraffin-based crayon. In some embodiments, the virtual gasket is patterned. For example, the hydrophobic coating can be applied in a pattern to the first substrate, the second substrate, or both. In some embodiments, the hydrophobic coating encompasses the biological sample, the array, or both. In some embodiments, the hydrophobic coating is applied in a similar pattern to the physical gaskets described herein. For example, the hydrophobic coating can have no apertures, one aperture, or two or more apertures. In some embodiments, the hydrophobic coating is extended beyond the encompassed biological sample, array, or both to prevent capillary flow between the first substrate and the second substrate. In some embodiments, the hydrophobic coating is applied patterned in a grid. For example, the hydrophobic coating can be applied (e.g., applied by any of the methods described herein) to encompass one or more biological samples, one or more arrays (e.g., spatial array), or both on a substrate. In some embodiments, the hydrophobic coating can be applied to encompass 2, 3, 4, 5, 6, 7, 8, 9, 10, 11, 12, or more biological samples, one or more arrays (e.g., spatial array), or both on a substrate (e.g., a first substrate, a second substrate). In some embodiments, the hydrophobic coating is patterning is controlled dynamically via electro wetting.

In some embodiments, a spacer is used to separate the two substrates (e.g., the first substrate and the second substrate). Spacers can be placed adjacent to the biological sample and in between the first substrate and the second substrate. By doing so, spacers can create a chamber in which solutions (e.g., a buffer, a permeabilization solution) are contained throughout the permeabilization and analyte migration process. In some embodiments, more than one spacer is used. In some embodiments, a spacer has a height of about 10 μm, about 12.5 μm, about 15 μm, about 17.5 μm, about 20 μm, about 22.5 μm, or about 25 μm. In some embodiments, the height of each spacer has a height of about 50 μm, about 100 μm, about 200 μm, about 300 μm, about 400 μm, about 500 μm, about 600 μm, about 700 μm, about 800 μm, about 900 μm, or about 1000 μm. In some embodiments, the spacer creates a fully enclosed chamber around the biological sample (e.g., tissue sample or a region of interest) and/or the array. The fully enclosed spacer can be any shape. In some embodiments, the fully enclosed (e.g., encompassed) spacer is one of a square or a rectangle. In some embodiments, the spacer conforms to the shape of the biological sample.

In some embodiments, the spacer partially encloses the biological sample (e.g., tissue or region of interest) or the array. In some embodiments, the spacer surrounds the biological sample on one, two, or three sides. In some embodiments, the spacer partially encloses the biological sample, enclosing approximately at least about 60%, at least about 70%, at least about 80%, at least about 90%, or at least about 95% of the surrounding biological sample.

In some embodiments, no spacer is used in the system and/or methods disclosed herein. In some embodiments, a spacer functions as a gasket as disclosed herein.

In some embodiments of any of the fluid delivery methods described herein, the gasket (e.g., any of the gaskets described herein, a spacer), including a virtual gasket, can be applied to a region of interest in a biological sample. In some embodiments, two or more gaskets, including two or more virtual gaskets, can be applied to 2, 3, 4, or more regions of interest in the biological sample. In some embodiments, a fluid (e.g., a permeabilization solution) can be delivered to 2, 3, 4, or more regions of interest where a gasket, including a virtual gasket, substantially encompasses a region of interest in the biological sample.

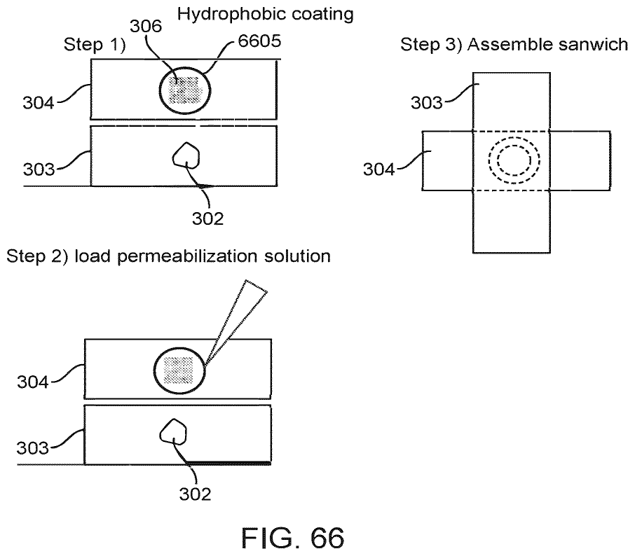

FIG. 66 shows an exemplary fluid delivery scheme including the use of a virtual gasket, (e.g., a hydrophobic coating 6605 as indicated by the arrow). As shown in Step 1 a hydrophobic coating 6605 can be applied to surround (e.g., encompass) the array 306, however, the hydrophobic coating 6605 can also be applied to surround the biological sample 302, or both. The hydrophobic coating 6605 can be applied via a stamp or drawn, such as for example, with a wax crayon or a paraffin-based crayon. As shown in Step 2 a fluid (e.g., permeabilization solution) is loaded on to the array 306 via capillary flow. While not shown, the fluid can be loaded via a syringe. Alternatively, dried permeabilization reagents can be disposed on the first substrate and/or the second substrate and can be solubilized by the delivered fluid. When the first substrate, the second substrate, and the virtual gasket (e.g., the hydrophobic coating 6605) are assembled (e.g., in a sandwich assembly, any of the configurations described herein) a partially sealed chamber if formed where fluid can be delivered (e.g., through an aperture, through a via-hole) to the partially enclosed volume of the chamber.

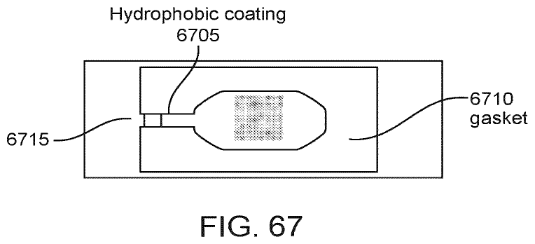

FIG. 67 shows a configuration combining a hydrophobic coating 6705 and a gasket 6710. As shown in FIG. 67 the gasket 6710 includes an aperture 6715. A hydrophobic coating 6705 can be applied to the substrate not covered by the gasket 6710 (e.g., in the aperture 6715), to retain the delivered fluid (e.g., a buffer, the permeabilization solution 305). The hydrophobic coating 6705 can be applied to the aperture (e.g., one or more apertures) prior to delivering the fluid.

In some embodiments, the fluid includes permeabilization reagents (e.g., any of the permeabilization reagents described herein). In some embodiments, the rate of permeabilization of the biological sample is modulated by delivering the permeabilization reagents (e.g., a fluid containing permeabilization reagents) at various temperatures. For example, the fluid (e.g., a permeabilization solution, a buffer) can be delivered from about 5° C. to about 80° C., from about 10° C. to about 75° C., from about 15° to about 70° C., from about 20° C. to about 65° C., from about 25° C. to about 60° C., from about 30° C. to about 55° C., from about 35° C. to about 50°, and from about 40° C. to about 45° C. In some embodiments, the permeabilization solution can be about 5° C., about 6° C., about 7°, about 8° C., 9°, about 10° C., about 11° C., about 12° C., about 13° C., about 14° C., about 15° C., about 16° C., about 17° C., about 18° C., about 19° C., about 20° C., about 21° C., about 22° C., about 23°, 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., or about 80° C.

In some embodiments, the permeabilization reagents are dried permeabilization reagents. In some embodiments, the dried permeabilization reagents are disposed on a substrate (e.g., the first substrate, the second substrate). In some embodiments, delivering the fluid (e.g., by any of the fluid delivery methods described herein) solubilizes the dried permeabilization reagents.

In some embodiments, controlling the temperate of the first substrate, the second substrate, or both, modulates permeabilization of the biological sample. For example, the first substrate, the second substrate, or both, can be disposed in a substrate holder (e.g., any of the substrate holders described herein). In some embodiments, heating the first substrate, the second substrate, or both includes heating the permeabilization solution (e.g., the fluid comprising permeabilization reagents, solubilized dried permeabilization reagents) and modulating permeabilization of the biological sample. For example, permeabilization can be actuated by heating once the system has equilibrated (e.g., after fluid delivery) and there is no flow present in the system. In some embodiments, cooling the first substrate, the second substrate, or both includes cooling the permeabilization solution (e.g., the fluid including permeabilization reagents, solubilized dried permeabilization reagents) and modulating permeabilization of the biological sample.

In some embodiments, the temperature of the first and second members is lowered to a first temperature that is below room temperature (e.g., 25 degrees Celsius) (e.g., 20 degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower). In some embodiments, the sample holder includes a temperature control system (e.g., heating and cooling conducting coils) that enables a user to control the temperature of the sample holder. Alternatively, in other embodiments, the temperature of the sample holder is controlled externally (e.g., via refrigeration or a hotplate). In a first step, the second member, set to or at the first temperature, contacts the first substrate, and the first member, set to or at the first temperature, contacts the second substrate, thereby lowering the temperature of the first substrate and the second substrate to a second temperature. In some embodiments, the second temperature is equivalent to the first temperature. In some embodiments, the first temperature is lower than room temperature (e.g., 25 degrees Celsius). In some embodiments, the second temperature ranges from about −10 degrees Celsius to about 4 degrees Celsius. In some embodiments, the second temperature is below room temperature (e.g., 25 degrees Celsius) (e.g., 20 degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower).

In some embodiments, controlling the temperate of the first substrate, the second substrate, or both modulates permeabilization of the biological sample includes heating to about 25° C. to about 55° C., to about 30° C. to about 50° C., to about 35° C. to about 45° C., or to about 40° C. In some embodiments, controlling the temperate of the first substrate, the second substrate, or both modulates permeabilization of the biological sample includes heating to about 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., or 55° C.

In some embodiments, the biological sample is permeabilized for a period of time. For example, the biological sample can be permeabilized from about 1 minute to about 90 minutes or any length of time in between, from about 5 minutes to 85 minutes, from about 10 minutes to about 80 minutes, from about 15 minutes to about 75 minutes, from about 20 minutes to about 70 minutes, from about 25 minutes to about 65 minutes, from about 30 minutes to about 60 minutes, from about 35 minutes to about 55 minutes, from about 40 minutes to about 50 minutes, or about 45 minutes. In some embodiments, the biological sample is permeabilized for about 30 minutes at 40° C.

In some embodiments, analytes that are released from the permeabilized tissue of the sample diffuse to the surface of the first substrate and are captured on the feature array (e.g., barcoded probes) of the second substrate. In a subsequent step, the first substrate and the second substrate are separated (e.g., pulled apart) and temperature control is stopped.

In some embodiments, the biological sample is imaged. In some embodiments, the biological sample is imaged on the first substrate prior to alignment with the second substrate. In some embodiments, the biological sample is a tissue section. In some embodiments, the biological sample is a fresh frozen biological sample. In some embodiments, the fresh frozen biological sample is a fresh frozen tissue section. In some embodiments, the biological sample is a fixed biological sample. In some embodiments, the fixed biological sample is a formalin-fixed paraffin-embedded biological sample.

In some embodiments, the array includes a plurality of features. In some embodiments, the array includes about 5,000 features. In some embodiments, a feature of the plurality of features is a bead. In some embodiments, a plurality of capture probes are attached to the bead. In some embodiments, the capture probe comprises a capture domain, and a spatial barcode unique to the feature. In some embodiments, the capture domain of the capture probe includes a poly(T) sequence. In some embodiments, the capture probe includes one or more functional domains, a cleavage domain, a unique molecular identifier, and combinations thereof.

### Fluid Delivery Kits

Also provided herein are kits including a first substrate including a coating (e.g., any of the coatings described herein) for adhering a biological sample, a second substrate comprising an array, and a spacer. Also provided herein are kits including a first substrate, the first substrate comprising a surface for adhering a biological sample, a second substrate comprising an array, and a spacer. In some kits, the spacer is disposed on the first substrate and/or the second substrate. In some kits, the spacer at least partially surrounds the biological sample and/or the array. In some kits, the spacer may be disposed between the first substrate and second substrate and configured to maintain a fluid within a chamber comprising the first substrate, the second substrate, the biological sample, and the spacer. The spacer may be further configured to maintain a separation distance between the first substrate and the second substrate. In some kits, the kit includes a paraffin-wax crayon. In some kits, the kit includes a hydrophobic coating stamp. In some kits, the kit includes a reverse transcriptase and a nuclease.

### Exemplary Workflows for Multiple Sandwich Closings and Multiple Permeabilization Steps

In some embodiments, the example workflows and the example sample handling apparatuses described herein may allow for quick and efficient spatial analysis for multiple tissue/biological samples. The workflows and the example workflows and the example sample handling apparatuses described herein may facilitate multiple sandwich closings between a first and a second substrate. In some aspects, the sandwich closings may be performed in series or in parallel to achieve spatial analysis of multiple tissue/biological samples.

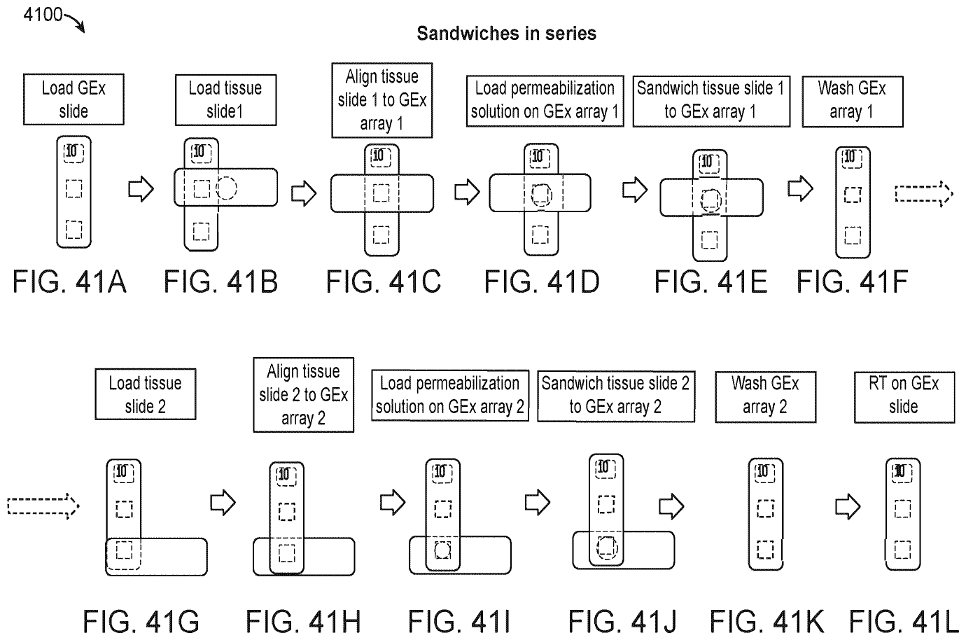

FIGS. 41A-41L depict an example workflow 4100 for providing multiple sandwich closings in series in accordance with example implementations. The sandwich closings described in FIGS. 41A-41L may be performed via any of the example sample handling apparatuses described herein (e.g., sample handling apparatus 400, sample handling apparatus 1400, sample handling apparatus 3500, or the like).

FIG. 41A depicts a second substrate (e.g., the slide 304, the second substrate 412, the second substrate 1412, a gene expression slide, or the like) loaded into a sample handling apparatus.

FIG. 41B depicts a first substrate (e.g., the pathology slide 303, the first substrate 406, the first substrate 1406, a tissue slide, or the like) loaded into the sample handling apparatus.

FIG. 41C depicts aligning the first substrate with the second substrate. The alignment may be performed by an alignment mechanism, an adjustment mechanism, a controller, or the like of the sample handling apparatus.

FIG. 41D depicts loading a permeabilization solution (e.g., permeabilization solution 305) on the second substrate. The permeabilization solution 305 may be positioned proximate to the capture probes 306 of the second substrate or any other region of the second substrate.

FIG. 41E depicts sandwiching the first substrate and the second substrate together with the permeabilization solution 305 disposed between the substrates. During the sandwiching, analytes and/or mRNA transcripts may release from the tissue sample 302 and be captured by the capture probes 306 of the second substrate.

FIG. 41F depicts washing the second substrate. Washing the second substrate may remove any residue sample material and/or permeabilization solution 305.

FIG. 41G depicts loading a subsequent first substrate into the sample handling apparatus. As shown, the second substrate may remain within the sample handling apparatus.

FIG. 41H depicts aligning the subsequent first substrate with the second substrate. The aligning may be performed by any means described herein.

FIG. 41I depicts loading the permeabilization solution 305 onto the second substrate. As shown, the permeabilization solution 305 may be loaded onto a second set of capture probes 306 of the second substrate.

FIG. 41J depicts sandwiching the subsequent first substrate with the second substrate. During this second sandwiching, the permeabilization solution 305 may cause analytes and/or mRNA transcripts to release from the tissue sample of the subsequent first substrate and be captured by the second set of capture probes 306 of the second substrate.

FIG. 41K depicts washing the second substrate. Washing the second substrate may include washing the second set of capture probes 306 to remove any residue tissue material and/or permeabilization solution.

FIG. 41L depicts performing reverse transcription or second strand synthesis on the second substrate for further analysis.

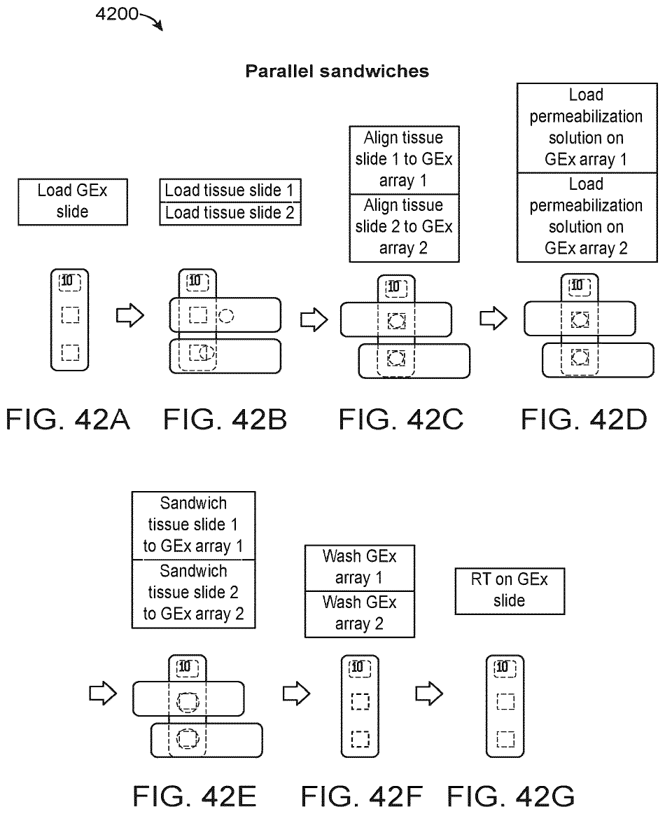

FIGS. 42A-42G depict an example workflow 4200 for providing multiple sandwich closings in parallel in accordance with example implementations. The sandwich closings described in FIGS. 42A-42G may be performed via any of the example sample handling apparatuses described herein (e.g., sample handling apparatus 400, sample handling apparatus 1400, sample handling apparatus 3500, or the like).

FIG. 42A depicts loading a second substrate (e.g., the slide 304, the second substrate 412, the second substrate 1412, a gene expression slide, or the like) into a sample handling apparatus. As shown, the second substrate includes a first array of capture probes 306 and the second array of capture probes 306.

FIG. 42B depicts loading a first tissue slide (e.g., a first substrate) and a second tissue slide (e.g., a second first substrate) into the sample handling apparatus. The first tissue slide and the second tissue slide may be arranged in parallel such as within a first member of the sample handling apparatus.

FIG. 42C depicts aligning the first tissue slide with the first array of capture probes 306 and aligning the second tissue slide with the second array of capture probes 306.

FIG. 42D depicts loading a permeabilization solution (e.g., permeabilization solution 305) onto the first array of capture probes 306 and onto the second array of capture probes 306 of the second substrate.

FIG. 42E depicts sandwiching the first tissue slide to the first array of capture probes 306 and sandwiching the second tissue slide to the second array of capture probes 306. During sandwiching, the first array may capture analytes and/or transcripts released from the first tissue slide and the second array may capture analytes and/or transcripts released from the second tissue slide.

FIG. 42F depicts washing the first array and the second array of the second substrate.

FIG. 42G depicts performing reverse transcription or second strand synthesis on the second substrate for further analysis.

In some embodiments, the workflows 4100 and/or 4200 can include applying permeabilization solutions more than once. In some embodiments, the permeabilization solution may be the same permeabilization solution that is applied more than once. In some embodiments, the permeabilization solution may include different permeabilization solutions that are each applied in a sequence of multiple permeabilization steps. Different assays can require different chemistries or experimental conditions, which can necessitate the need for different permeabilization solutions.

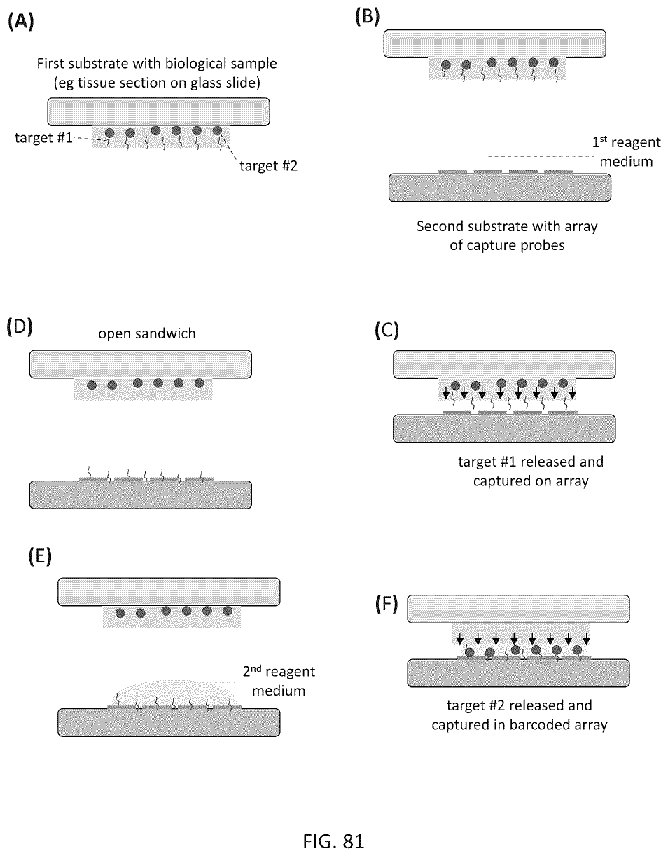

FIG. 81 depicts an exemplary method of capturing analytes from a biological sample with aid of a sample handling device disclosed herein. The method may comprise a step (A) mounting a first substrate comprising the biological sample on a first member of the sample handling device (not shown). The method may comprise a step (B) of mounting a second substrate on a second member of the sample handling device (not shown). The second substrate may comprising an array of capture probes, wherein a first capture probe of the array of capture probes comprises a first spatial barcode sequence and a first capture domain and wherein a second capture probe of the array of capture probes comprises a second spatial barcode sequence and a second capture domain. While (B) depicts the second substrate with a first reagent medium deposited, it is understood that the second substrate may in some cases not comprise the reagent medium deposited thereon. In such cases the method may further comprise the step of applying the first reagent medium to the first substrate and/or the second substrate. The first reagent medium may be configured to release a first analyte from the biological sample. The method may comprise a step (C) of moving the first member and/or the second member such that at least a first area of the biological sample and a first capture probe of the array are fluidically coupled via the first reagent medium (e.g., when the first and second substrates are arranged in a sandwich configuration in a first sandwiching step). As shown in (C), the first analyte is released and then captured on the array. The method may comprise a step (D) of moving the first member and/or the second member to fluidically decouple the first area of the biological sample and the first capture probe (e.g., by opening the sandwich). The method may comprise a step (E) of applying a second reagent medium to the first substrate and/or the second substrate, the second reagent medium configured to release a second analyte from the biological sample. The method may comprise a step (F) of moving the first member and/or the second member such that at least the first area of the biological sample and the second capture probe are fluidically coupled via the second reagent medium (e.g., when the first and second substrates are arranged in a sandwich configuration in a second sandwiching step. As shown in (F), the second analyte is released and captured on the array.

In some embodiments, the alignment mechanism of the sample handling apparatus 400, 1400, 3500 can maintain alignment between sample substrates and array substrates during workflows requiring multiple permeabilization steps, thus ensuring that the same spatial area of the biological sample is aligned with the same array location during the multiple permeabilizations. In some embodiments, referring to FIG. 81, the first spatial barcode sequence and the second spatial barcode sequence are identical. In some embodiments, the first capture probe is located in the same array feature as the second capture probe. Further details on the features of an exemplary array are disclosed in WO2020123320, which is incorporated by reference herein.

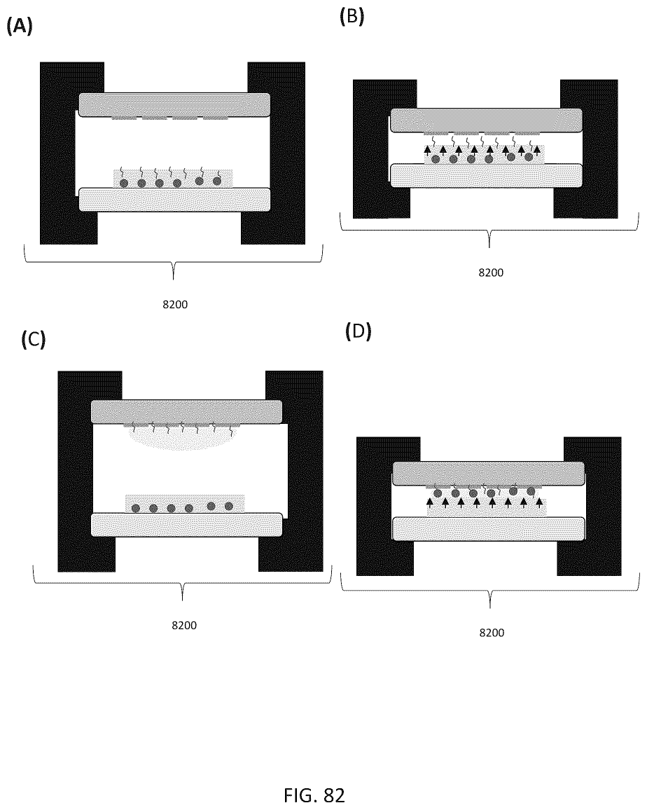

FIG. 82 depicts another exemplary method wherein the first and second substrates are mounted in the sample handing device (A), the first reagent medium is applied (not shown), the first and/or second substrates are moved toward one another to fluidically couple the at least first area of the biological sample with the first capture probe of the array (B), the first and/or second substrates are moved to fluidically decouple the first area of the biological sample and the first capture probe (C), applying the second reagent medium, and the first member and/or the second member are moved such that the first area of the biological sample and the second capture probe are fluidically coupled via the second reagent medium (D), thereby releasing the second analyte from the first area of the biological sample, wherein the released second analyte binds to the second capture domain. As shown in FIG. 82, a mechanical fixture can prevent or minimize XY movement during the decoupling step (C) and the moving step (D). In some embodiments, the mechanical fixture 8200 is manually adjustable. In some embodiments, the mechanical fixture 8200 is adjustable via a controller of the sample handling device.

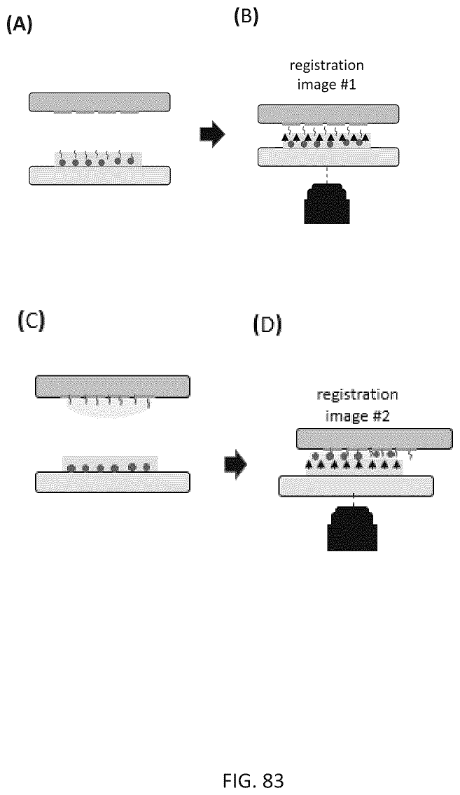

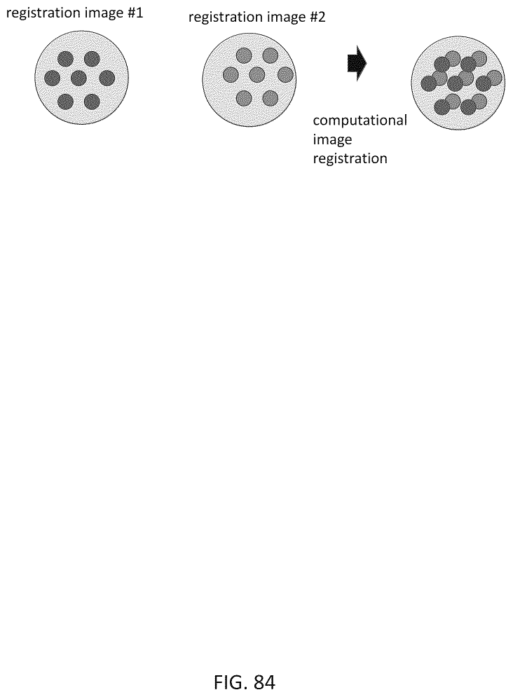

In some cases, the first and second substrates might not maintain XY alignment during the multiple permeabilizations. In such cases, the image capture device 2120 of the sample handling apparatus can be utilized in conjunction with image registration techniques to correct the alignment computationally. Referring to FIG. 83, the first and second substrates are mounted in the sample handing device (A), the first reagent medium is applied (not shown), the first and/or second substrates are moved toward one another to fluidically couple the at least first area of the biological sample with the first capture probe of the array (B), the first and/or second substrates are moved to fluidically decouple the first area of the biological sample and the first capture probe (C), applying the second reagent medium, and the first member and/or the second member are moved such that the first area of the biological sample and the second capture probe are fluidically coupled via the second reagent medium (D), thereby releasing the second analyte from the first area of the biological sample, wherein the released second analyte binds to the second capture domain. In some embodiments, responsive to (B), an image capture device of the sample handling device acquires first image data comprising a first overlay of the first area of the biological sample with the first area of the array. The image capture device may, responsive to (D), further acquire second image data comprising a second overlay of the first area of the biological sample with the second area of the array within the capture domain. As shown in FIG. 83, a shift in XY alignment between the first and second substrate occurred in (D). In such circumstances, the first spatial barcode sequence of the first capture probe and the second spatial barcode sequence of the second capture probe are different. A first area of the array (e.g., a first feature) may comprise the first capture probe and a second area of the array (e.g., a second feature) may comprise the second capture probe. Referring to FIG. 84, an image registration approach can advantageously correct the alignment computationally such that both the first and second spatial barcode sequences are associated with the same first area of the biological sample. The image registration can be used to register the first image data and the second image data, and generate an aligned imaged based on the registering, the aligned image comprising an overlay of the first area of the array with the second area of the array.

In some embodiments, moving the first member/and or the second member to fluidically couple the first area of the biological sample and the first capture probe of the array comprises moving the first member towards the second member, moving the second member towards the first member, or moving the first and second members towards each other until a separation distance between first and second substrates is achieved and maintained.

Exemplary separation distances are disclosed herein.

In some embodiments, moving the first member/and or the second member to fluidically decouple the first area of the biological sample and the first capture probe of the array comprises moving the first member away from the second member, moving the second member away from the first member, or moving the first and second members away from each other such that the separation distance is no longer maintained.

In some embodiments, the first and/or second reagent medium may comprise a permeabilization agent. The first reagent medium may comprise a first agent for releasing a first analyte from the biological sample, and the second reagent medium may comprise a second agent for releasing a second analyte from the biological sample. The first analyte may be a different type of analyte than the second analyte. Analyte types can include, without limitation, RNA, DNA, protein or polypeptide, RNA templated ligation products (described in, e.g., WO2021133849A1, which is hereby incorporated by reference in its entirety), and analyte capture agents (e.g., oligo-conjugated antibodies (or portions thereof), as described in, e.g., WO2021133849A1).

In particular embodiments, the first reagent medium does not comprise a permeabilization agent and the second reagent medium comprises a permeabilization agent. In other cases, the first reagent medium comprises a lower concentration of the permeabilization agent as compared to the second reagent medium. For example, in cases where the first analyte type is an analyte capture agent (e.g., oligo-conjugated antibody), the oligonucleotide conjugated to the antibody may comprise a cleavage site, e.g., a site specific cleavage site, such as a restriction enzyme recognition sequence. In particular embodiments wherein the oligonucleotide comprises a restriction enzyme recognition sequence, the first agent may be the cognate restriction enzyme. In such cases, the first sandwiching step releases the oligonucleotide from the antibody for capture by the array. The second sandwiching step with the second reagent medium may then permeabilize the sample thereby releasing the second analyte type (e.g., RNA, DNA, RNA templated ligation product).

In some aspects, the example sample handling apparatuses described herein may implement software to provide some of the functions of the sample handling apparatus. For example, software may be used to control aspects of image processing, substrate alignment, substrate temperature control, instrument safety, or the like.

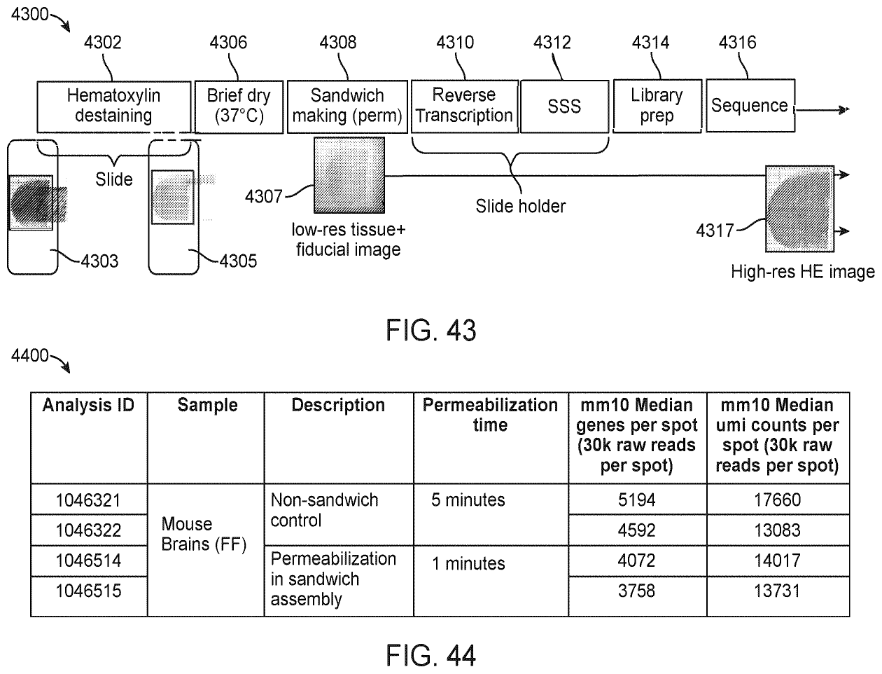

FIG. 43 depicts a workflow 4300 for performing sample analysis in accordance with example implementations. As shown, the workflow 4300 includes a destaining step 4302, a drying step 4306, a sandwich making (e.g., permeabilization) step 4308, a reverse transcription step 4310, a second strand synthesis (SSS) 4312, a library prep step 4314, and a sequence step 4316. The destaining step 4302 may include a hematoxylin destaining of a tissue slide. As further shown, the step 4302 may include an initial stained tissue slide 4303 and, after a destaining process, the destained tissue slide 4305. At the drying step 4306, the tissue slide 4305 may be dried for a duration at 37° C. or another temperature. At step 4308, the tissue slide 4305 may be sandwiched in a sample handling apparatus with a gene expression slide (e.g., slide 304 with barcoded array capture probes 306) and permeabilization medium.

In some aspects, during the step 4308, the sample handling apparatus may capture an image 4307 of the tissue section. The image may include a low resolution image of the tissue section and any fiducial on the tissue slide. At step 4310, the workflow 4300 may include performing reverse transcription on the second substrate. Step 4312 may include performing second strand synthesis on the second substrate. At step 4314, the workflow 4300 may include generating a cDNA library associated with a particular spatial barcode of the gene expression slide. The sequence step 4316 may include library amplicons may be sequenced and analyzed to decode spatial information. Barcoded cDNA libraries may be mapped back to a specific spot on a capture area of the capture probes 306. This gene expression data may be subsequently layered over a high-resolution microscope image of the tissue section. In some aspects, during or before sequencing, a high resolution image 4317 of the tissue section may be captured for the layering described above.

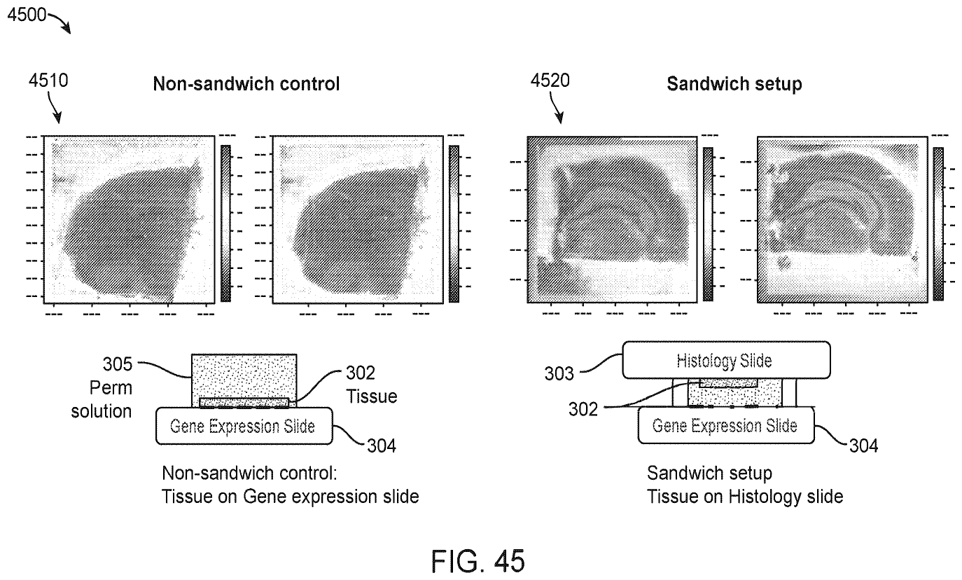

FIG. 44 depicts a table 4400 of example parameters of the workflow 4300 in accordance with example implementations. As shown, the table 4400 includes six columns and four rows depicting various characteristics of a tissue sample and analysis. For example, the table 4400 identifies the tissue sample as mouse brains, includes a description of the sandwich making step 4308 (e.g., the foreman sandwich configuration versus the control non-sandwich configuration), includes a permeabilization time, a media genes per spot (e.g., capture probe), and a median unique molecular identifiers (UMI) counts per spot.

FIG. 45 depicts a comparison between a non-sandwich control permeabilization step 4510 and a sandwich configuration permeabilization step 4520. As shown in the bottom left of FIG. 45, in the non-sandwich configuration, a tissue sample 302 is disposed on a gene expression slide 304 and a permeabilization solution 305 is applied on top of the slide 304. As shown in the bottom right hand side of FIG. 45, in the sandwich configuration, a tissue sample 302 is disposed on a slide 303 and sandwiched between the gene expression slide 304. As shown, the permeabilization solution 305 creates a permeabilization buffer between the slides 303 and 304 and facilitates target molecule and analyte capture on the gene expression slide 304. The top portion of FIG. 45 depicts example images captured of the tissue sample 302.

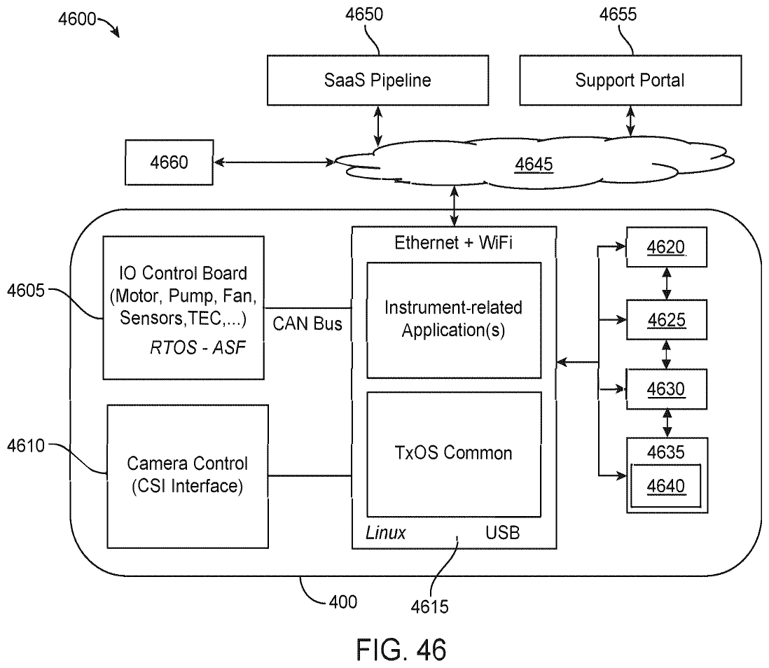

FIG. 46 is a diagram of an example system architecture 4600 in accordance with some example implementations described herein. For example, the system architecture 4600 can be configured to perform one or more of workflows and processes described herein. As shown in FIG. 46, the sample handling apparatus 400 may include an input/output control board 4605, a camera control 4610, and a network interface 4620. As shown, the input/output control board 4605, the camera control 4610, and the network interface 4615 may be connected via a controller area network (CAN) bus. The input/output control board 4605 may be configured to control aspects or components of the sample handling apparatus 400. For example, the input/output control board 4605 can include a controller and may be configured to control a pump, a fan, a motor of a linear actuator, one or more sensors, a heater, a TEC, or the like. In some embodiments, the input/output control board 4605 can control operation of one or more actuators, illumination sources, fluid sources, or the like that can be configured within the sample handling apparatus 400. The camera control 4610 may be configured to control aspects or components of a camera (e.g., the image capture device 2120). For example, the camera control 4610 may control a focus, a zoom, a position of the camera, an image capture, or the like. In some embodiments, the camera control 4610 can control a focus motor 3530 that is coupled to or integrated within the image capture device 2120.

The sample handling apparatus 400 also includes a processor 4620, a memory 4625, an input device 4630, and a display 4635. The processor 4620 can be configured to execute computer-readable instructions stored the memory 4625 to perform the workflows and processes described herein. The processor 4620 can also execute computer-readable instructions stored in the memory 4625, which cause the processor 4620 to control operations of the sample handling apparatus 400 via the I/O control board 4605 and/or the image capture device 2120 via the camera control 4610. In this way, the processor 4620 can control an operation of the sample handling apparatus 400 to align a sample with an array. For example, the processor 4620 can execute instructions to cause either of the first retaining mechanism or the second retaining mechanism to translate within the sample handling apparatus 400 so as to adjust their respective locations and to cause a sample area of a first substrate to be aligned with an array area of a second substrate.

The input device 4630 can include a mouse, a stylus, a touch-pad, a joy stick, or the like configured to receive user inputs from a user. For example, a user can use the input device 4630 to provide an input indicating a sample area indicator for a first substrate. The display 4635 can include a graphical user interface 4640.

The network interface 4615 may be configured to provide wired or wireless connectivity with (e.g., via Ethernet, Wi-Fi, or the like) a network 4645, such as the Internet, a local area network, a wide area network, a virtual private network, or the like. The network 4645 may be connected to one or more distributed computing resources, such as a cloud computing environment, a software as a service (SaaS) pipeline 4650, and/or a support portal 4655. The SaaS pipeline 4650 may be configured to aid or control automated image alignment or other alignment. The support portal 4655 may be configured to send images/videos/logs to the support portal and for issues to debug. The sample handling apparatus 400 can also be communicatively coupled via the network 4645 to a second computing device 4660 located remotely from the location of the sample handling apparatus 400.

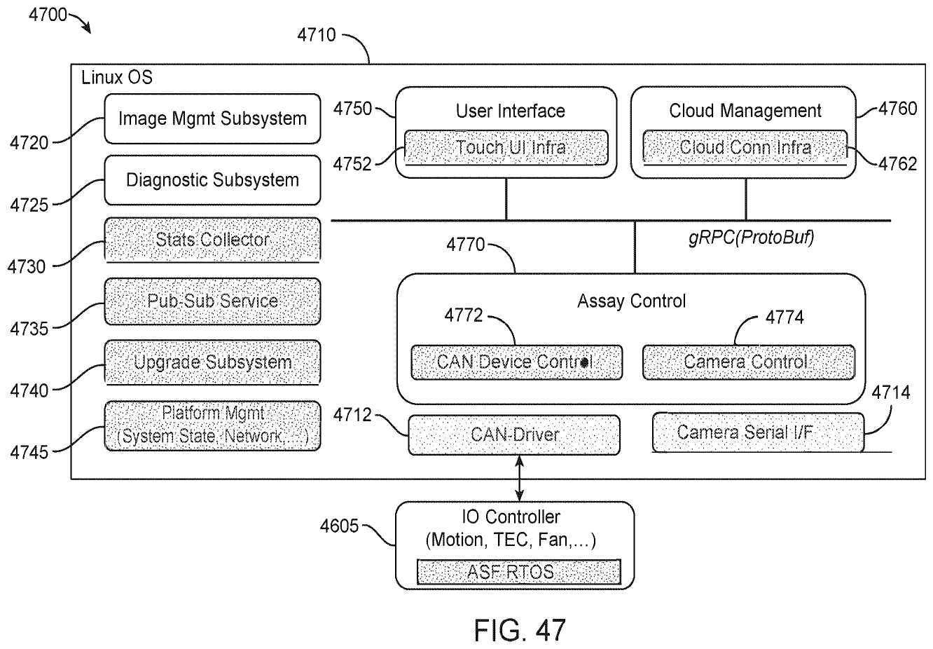

FIG. 47 is a diagram of a sample handling apparatus software building block 4700 in accordance with some example implementations. As shown, the input/output controller 4605 may be connected to an operating system (e.g., Linux OS) 4710. The operating system 4710 may include an image management subsystem 4720, a diagnostic subsystem 4725, a statistics collector 4730, a publication and subscription service 4735, and upgrade subsystem 4740, a platform management subsystem 4745, a user interface subsystem 4750, a cloud management subsystem 4760, and an assay control subsystem 4770. The user interface subsystem 4750 may include a touchscreen user interface infrastructure 4752. The cloud management subsystem 4760 may include a cloud connectivity infrastructure 4762. The assay control subsystem 4770 may include a controller area network (CAN) device control subsystem 4772 and a camera control subsystem 4774. The operating system 4710 may further include a CAN driver 4712 and a camera serial interface 4714. The CAN device control subsystem 4772 may connect to other boards controlling other sensors, actuators, or other components. The camera serial interface 4714 may be configured to control and record images/videos using the image capture device(s). In some embodiments, one or more portions of the assay control subsystem 4770 can be configured with computer readable and executable instructions controlling an illumination source provided in (or otherwise coupled to) the sample handle apparatus described herein.

### Exemplary Aspects of Sample Handling Apparatuses

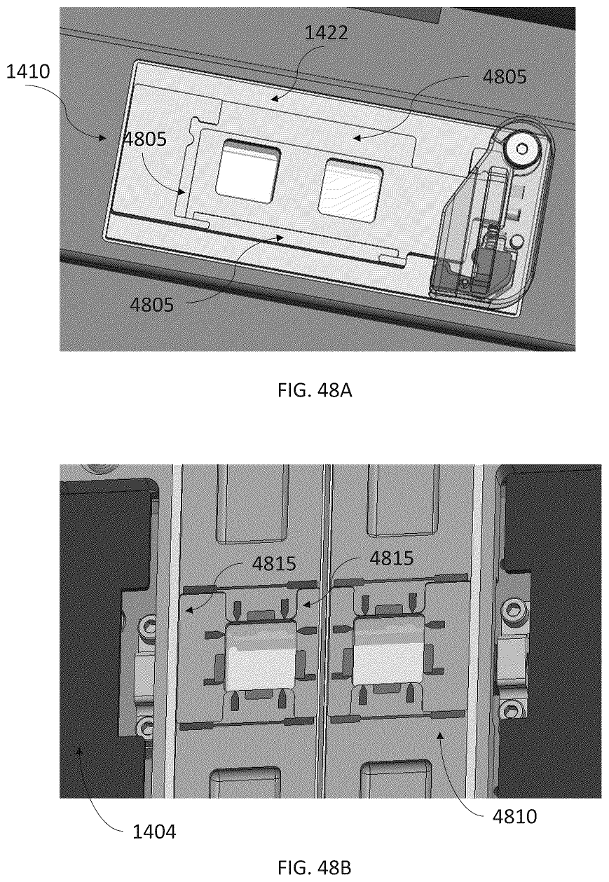

FIG. 48A depicts a top view of one embodiment of troughs configured in a retaining mechanism of a sample handling apparatus described herein in accordance with some example implementations. With reference to FIGS. 14A-B, the sample handling apparatus 1400 can include a second member 1410. The second member 1410 can include a retaining mechanism, such as the second retaining mechanism 1422 onto which an array substrate can be received and secured. The retaining mechanism 1422 can include a trough or cavity 4805 extending around an area of the retaining mechanism 1422 upon which a substrate is received. The trough 4805 can be a step-down trough such that it is configured on and below a surface at which the second substrate is received in the retaining mechanism 1422.

The trough 4805 can prevent excess media supplied during permeabilization from entering gaps between a slide and the retaining mechanism 1422. The trough 4805 can form a gap that can suppress capillary flow pumping and reduce fluid movement during application of a reagent or a permeabilization solution and the entire assay time as described herein. The trough 4805 can advantageously suppress and manage the flow of fluid reagents used in the sample handling apparatus 1400.

FIG. 48B depicts a top view of another embodiment of troughs configured in a retaining mechanism of a sample handling apparatus described herein in accordance with some example implementations. The retaining mechanism 4810 shown in FIG. 48B can correspond to the first retaining mechanism 1408 shown and described in relation to FIG. 14B. One or more substrates, such as substrates 1406, can be received and secured within the retaining mechanism 1408. A trough 4815 can be configured on the retaining mechanism 1408. The trough 4815 can extend around an area of the retaining mechanism upon which the one or more substrates 1406 are received. The trough 4815 can be a step-down trough such that it is configured on and below a surface at which the substrates 1406 are received in the retaining mechanism 1408.

The trough 4815 can prevent excess media supplied during permeabilization from entering gaps between a slide and the retaining mechanism 1408. The trough 4815 can form a gap that can suppress capillary flow pumping and reduce fluid movement during application of a reagent or a permeabilization solution and the entire assay time as described herein. The trough 4815 can advantageously suppress and manage the flow of fluid reagents used in the sample handling apparatus 1400.

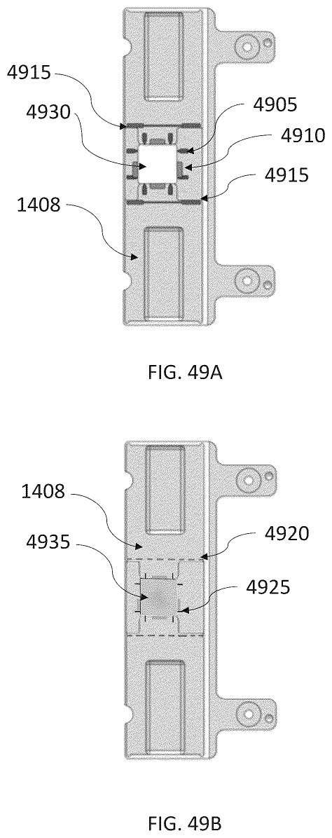

FIG. 49A depicts a top view of an embodiment of alignment marks configured on a retaining mechanism of a sample handling apparatus described herein in accordance with some example implementations. The retaining mechanism 1408 shown in FIG. 49A can correspond to the first retaining mechanism 1408 shown and described in relation to FIG. 14B. The retaining mechanism 1408 can include one or more alignment marks, such as alignment marks 4905, 4910 and 4915. The alignment marks can be provided to aid a user in aligning a substrate, such as a substrate including a sample thereon. The alignment marks can provide a visual cue to a user for engaging the substrate with the retaining mechanism 1408 so that a portion of a sample is correctly located on the retaining mechanism 1408.

In some embodiments, the retaining mechanism 1408 can include alignment marks 4905. The alignment marks 4905 can be provided for substrates with larger sample areas, such as an 11 mm×11 mm sample area. In some embodiments, the retaining mechanism can include alignment marks 4910. The alignment marks 4910 can be provided for a second substrate with a second sample area size, such as a 6.5 mm×6.5 mm sample area size. In some embodiments, the retaining mechanism can include alignment marks 4915. The alignment marks 4915 can indicate an exclusion zone in which a sample on a substrate should not be located. In some embodiments, the alignment marks 4915 can define an exclusion zone associated with raised or elevated substrate features, such as a label or one or more frosted portions of a substrate. In some embodiments, the alignment marks 4905, 4910, and/or 4915 can include various colors, shapes, and/or line patterns.

FIG. 49B depicts of another embodiment of alignment marks configured on a retaining mechanism of a sample handling apparatus described herein in accordance with some example implementations. As shown in FIG. 49B, a variety of visual designs can be applied to represent alignment markers. For example, alignment marker 4920 can include a dashed line to indicate a no-go zone. The alignment marks can vary in size and shape. In some embodiments, the alignment markers 4925 can be oriented relative to geometric features of a viewing window 4930 formed in the retaining mechanism 1408, as shown FIG. 49A (and which is obscured by sample 4935 in FIG. 49B). In some embodiments, the alignment markers 4920 and/or 4925 can include various colors, shapes, and/or line patterns.

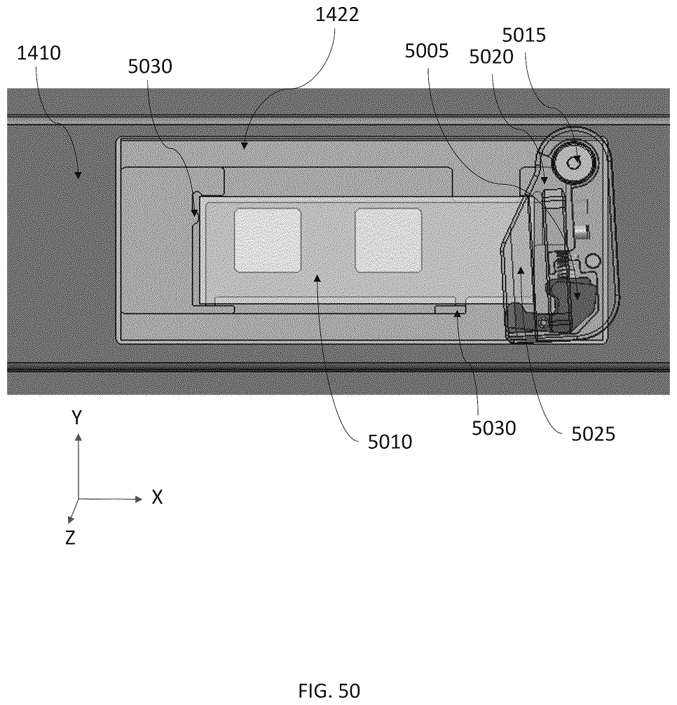

FIG. 50 depicts an alignment clip configured on a retaining mechanism (array substrate holder) of a sample handling apparatus described herein in accordance with some example implementations. As shown in FIG. 50, a member, such as second member 1410 can include a retaining mechanism, such as a second retaining mechanism 1422. The retaining mechanism 1422 can include an alignment clip 5005 configured within a clip housing 5020. The alignment clip 5005 can be actuated by a user to secure a substrate 5010 to or within the retaining mechanism 1422. The alignment clip 5005 can be configured to initially align the substrate 5010 within the retaining mechanism 1422 along the Y-axis and can subsequently align the substrate 5010 along the X-axis. In some embodiments, the alignment clip 5005 can be configured to initially align the substrate 5010 within the retaining mechanism 1422 along the X-axis and can subsequently align the substrate 5010 along the Y-axis. The alignment clip 5005 can be actuated with respect to a pivot axis point 5015. In some embodiments, the pivot axis point 5015 can be configured in any corner or portion of the retaining mechanism 1422 and is not limited to the configuration shown in FIG. 50.

As further shown in FIG. 50, the alignment clip 5005 includes an overhang portion 5025. The overhang portion 5025 can overhang or cover a portion of the substrate 5010. The overhang portion 5025 can limit the substrate 5010 from moving with respect to the Z-axis.

The retaining mechanism 1422 can include one or more datums 5030 at one or more locations on the retaining mechanism 1422. The datums 5030 can be protrusions configured to abut an edge of the substrate 5010 when placed within the retaining mechanism 1422. The datums 5030 can be configured with respect to the retaining mechanism 1422 and the substrate 5010 such that an equal amount of force is applied to each datum when the alignment clip 5005 engages the substrate 5010. In some embodiments, the retaining mechanism 1422 can include one or more datums 5030 on the X-axis (e.g., the longer, horizontally oriented side of the substrate 5010 as shown in FIG. 50) and/or one or more datums 5030 on the Y-axis (e.g., the short, vertically oriented side of the substrate 5010 as shown in FIG. 50).

In some embodiments, the retaining mechanism 1422 and the alignment clip housing 5020 can include one or more magnets. The magnets can be arranged in an offset manner, such that the magnets affect a split bias force as the alignment clip 5005 is articulated about the pivot axis point 5015. In some embodiments, a first magnet in the retaining mechanism 1422 can be vertically offset (e.g., along the Z-axis shown in FIG. 50) from a second magnet included in the alignment clip housing 5020. In some embodiments, the first magnet can be offset from the second magnet by 1-45 degrees relative to the pivot axis point 5015. In a preferred embodiment, the first magnet can be offset from the second magnet by 1-15 degrees relative to the pivot axis point 5015. For example, the first magnet can be offset from the second magnet by about 1 degree, about 2 degrees, about 3 degrees, about 4 degrees, about 5 degrees, about 6 degrees, about 7 degrees, about 8 degrees, about 9 degrees, about 10 degrees, about 11 degrees, about 12 degrees, about 13 degrees, about 14 degrees, or about 15 degrees. The first magnet can exert a repelling force against the second magnet when the substrate 5010 is retained by the retaining mechanism 1422. In this way, due to the offset arrangement, the magnets of the retaining mechanism 1422 and the alignment clip housing 5020 are repelled from one another when closing the alignment clip 5005 onto the substrate 5010 and are attracted to one another when the alignment clip 5005 is disengaged from the substrate 5010. The split bias design can provide enhanced usability for users manipulating the alignment clip 5005 with regard to the substrate 5010 and can also aid to cause the alignment clip 5005 to come into engagement with the substrate 5010.

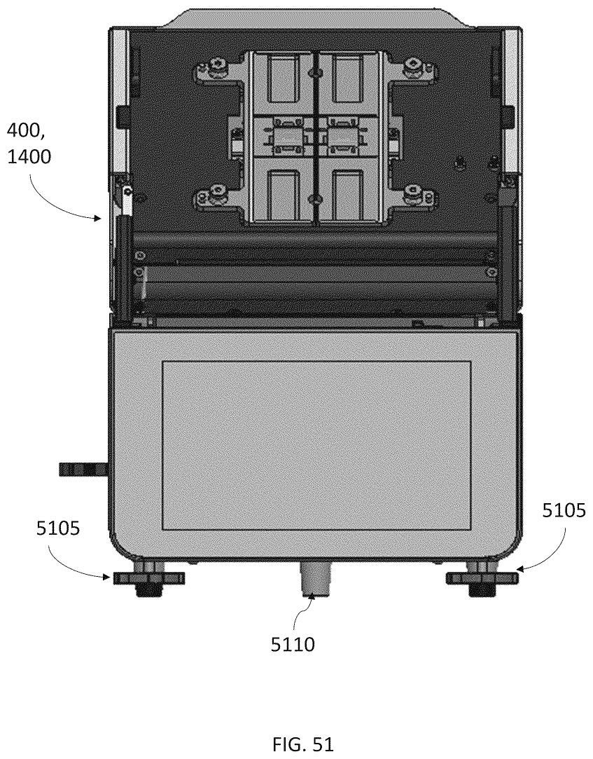

FIG. 51 depicts one or more leveling mechanisms configured on a sample handling apparatus described herein in accordance with some example implementations. As shown in FIG. 51, leveling mechanisms 5105 can be configured on a bottom surface of a sample handling apparatus, such as the sample handling apparatus 400 or 1400 described herein. The leveling mechanisms 5105 can be adjusted by a user to level the sample handling apparatus 400, 1400 with respect to a surface on which it is placed. For example, the leveling mechanisms 5105 can include a threaded, screw-type leveling mechanisms that can be rotated to raise or lower a portion of the sample handling apparatus 400, 1400. In some embodiments, the sample handling apparatus 440, 1400, and 3500 can include one or more non-adjustable feet 5110. The leveling mechanisms 5105 can be configured to adjust the sample handling apparatus 400, 1400, 3500 within +/−1.5 degrees of level relative to a surface on which it is located.

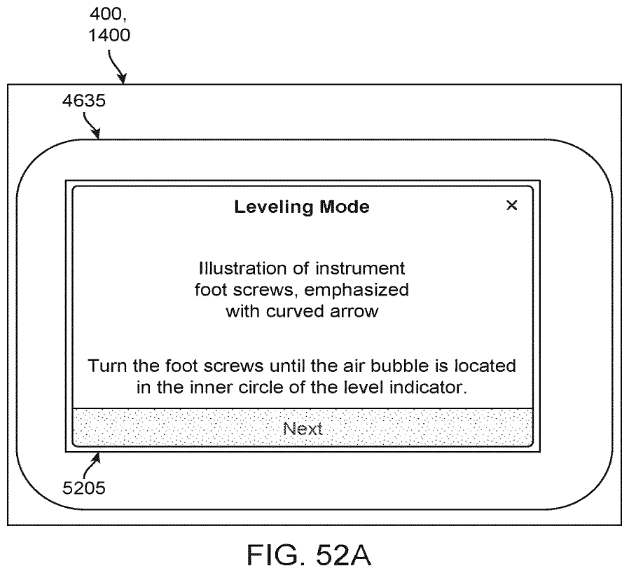

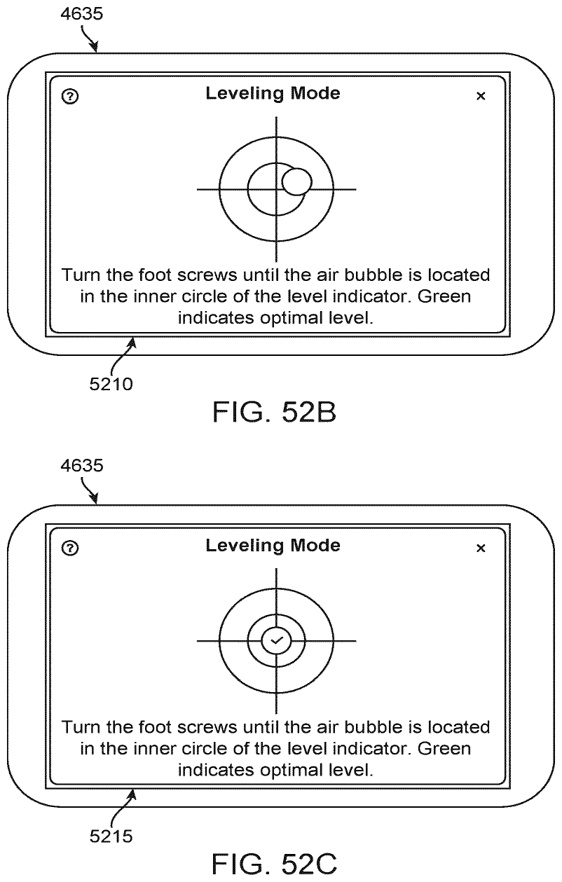

FIGS. 52A-52C depict user interfaces associated with the one or more leveling mechanism configured on a sample apparatus described herein in accordance with some example implementations. As shown in FIG. 52A, the sample handling apparatus 400, 1400 can include a display, such as display 4635 described in relation to FIG. 46. The display 4640 can be configured to provide one or more graphical user interfaces 4640 for display. In some embodiments, the display 4635 can provide a leveling mode interface 5205. The leveling mode interface 5205 can instruct and provide feedback to a user when leveling the sample handling apparatus 400, 1400 using the leveling mechanisms 5105. As shown in FIG. 52B, the display 4635 can provide a leveling adjustment interface 5210. The leveling adjustment interface 5210 can include a visualization of an air bubble that can move along a level indicator (e.g., a cross-hair surrounded by one or more concentric circles) when adjusting the level via the leveling mechanisms 51505. The user can receive instructions to adjust the leveling mechanisms 5105 until the air bubble is located in the inner circle of the level indicator. As shown in FIG. 52C, the display 4635 can provide a level confirmation interface 5215. The level confirmation interface 5215 can indicate that the leveling performed by the user has achieved a desired state and that the sample handling apparatus 400, 1400 is level with respect to a surface on which it is located.

The leveling interfaces 5205, 5210, and 5215 can be provided in the display 4635 when the sample handling apparatus is in leveling mode.

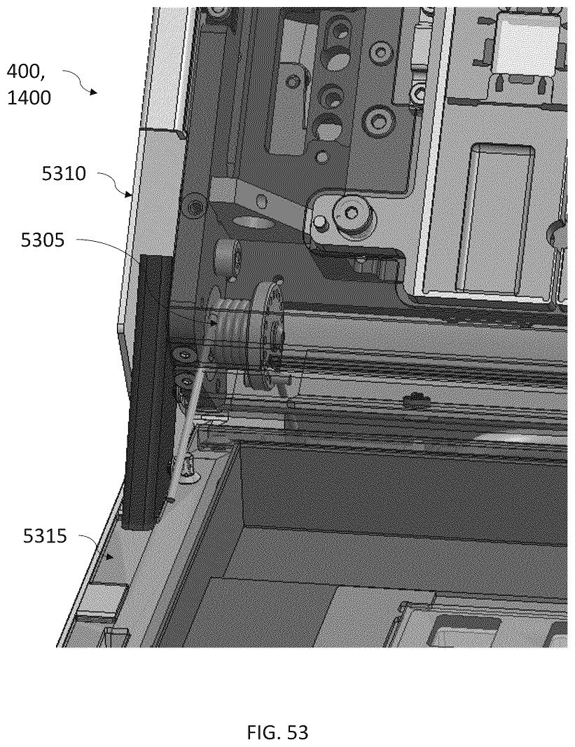

FIG. 53 depicts a hinge resistance mechanism configured on a sample handling apparatus described herein in accordance with some example implementations. The hinge resistance mechanism 5305 can be configured with respect to hinge 1415 described in relation to FIG. 14. The hinge resistance mechanism 5305 can be configured as a spring located on one or both sides of atop portion 5310 of the sample handling apparatus 400, 1400 described herein. The hinge resistance mechanism 5305 can provide resistance when closing the top portion 5310 (e.g., first member) of the sample handling apparatus onto the bottom portion 5315 (e.g., second member of the sample handling apparatus). The hinge resistance mechanism 5305 can provide assistance when opening the top portion 5310 of the sample handling apparatus from the bottom portion 5315. In some embodiments, a friction hinge damper can be configured for operation with the hinge resistance mechanism 5305. For example, the friction hinge damper can be configured opposite to a spring hinge forming the hinge mechanism 5305. In some embodiments, a plurality of friction dampers can be configured in the sample handling apparatus 400, 1400, 3500 described herein. The friction hinge damper can be configured to provide resistance when closing atop portion 5310 with the bottom portion 5315. The friction hinge damper can also provide assistance when opening the top portion 5310 from the bottom portion 5315. In this way, the friction hinge damper can prevent the top portion 5315 from uncontrolled or excessive travel when closing or opening the top portion 5310 relative to the bottom portion 5315.

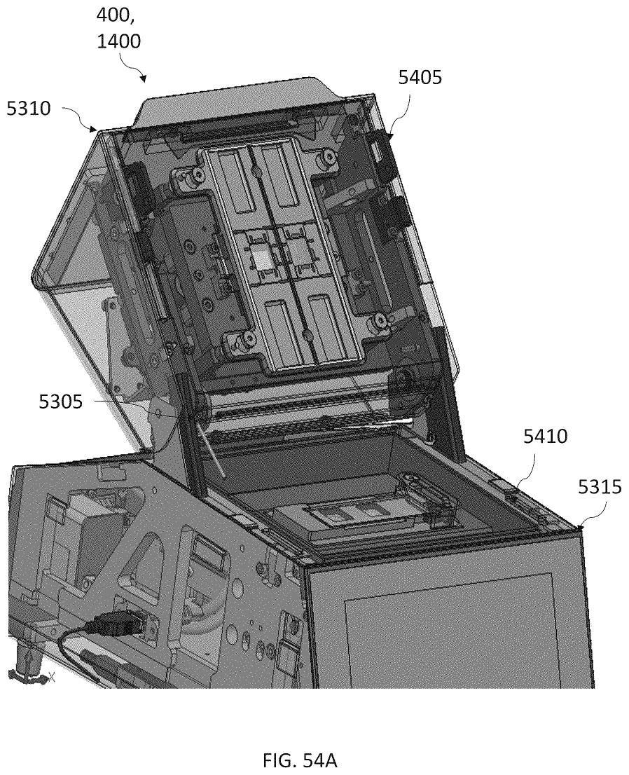

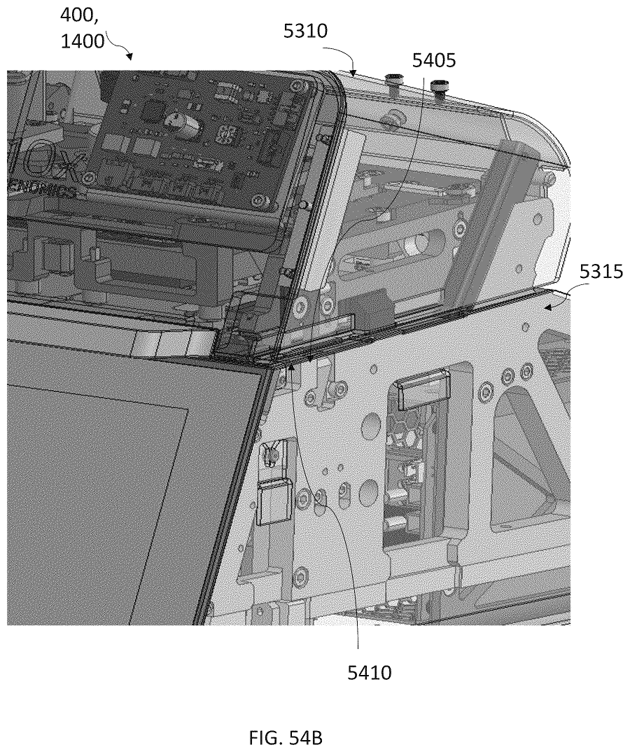

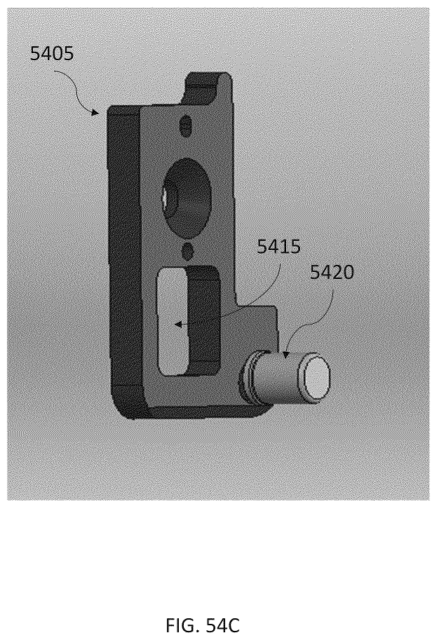

FIGS. 54A-54C depict a closure feedback mechanism configured on a sample handling apparatus described herein in accordance with some example implementations. As shown in FIG. 54A, the top portion 5310 of the sample handling apparatus 400, 1400 can include a closure feedback mechanism 5405. As shown in FIG. 54B, the closure feedback mechanism 5405 can be received in a receiving portion 5410 configured in the bottom portion 5315 of the sample handling apparatus 400, 1400. The receiving portion 5410 can include a slot to receive the closure feedback mechanism 5405. As shown in FIG. 54C, the closure feedback mechanism 5405 can include a hole 5415 that can be received within and engage with the receiving portion 5410 to secure the top portion 5310 and the bottom portion 5315 together. The closure feedback mechanism 5405 can be secured to the top portion 5310 via a pin 5420.

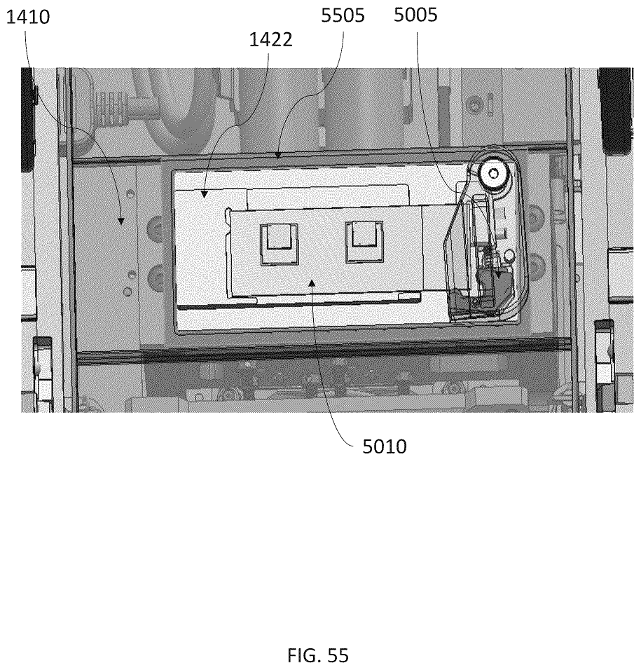

FIG. 55 depicts a gasket configured on a retaining mechanism of a sample handling apparatus described herein in accordance with some example implementations. As shown in FIG. 55, the retaining mechanism 1422 can retain a substrate, such as substrate 5010 on which an array can be provided. A gasket 5505 can be configured around the retaining mechanism 1422. The gasket 5505 can prevent fluid, such as reagent fluid or permeabilization solution, from flowing off the retaining mechanism 1422 and into the sample handling apparatus 400, 1400. In some embodiments, the gasket 5505 can be formed from a rubber material or a polymer material.

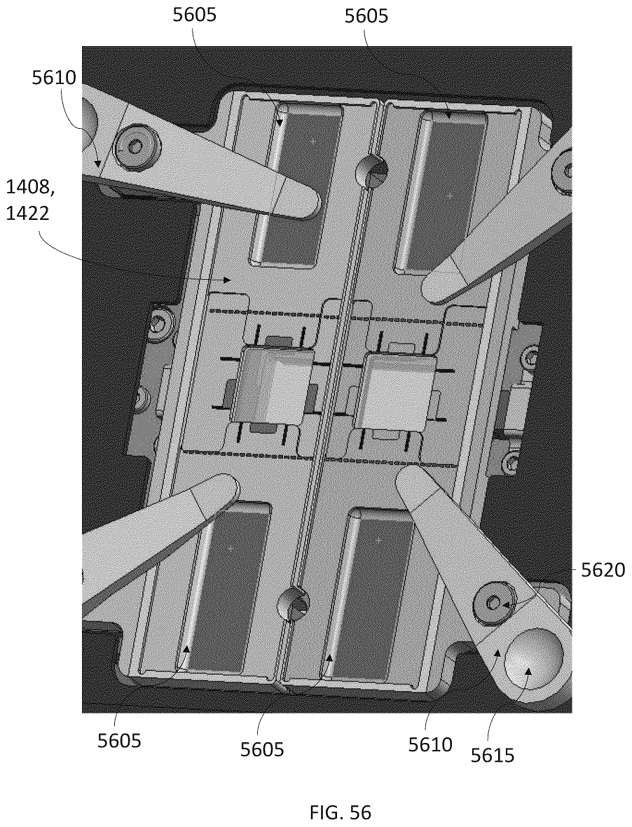

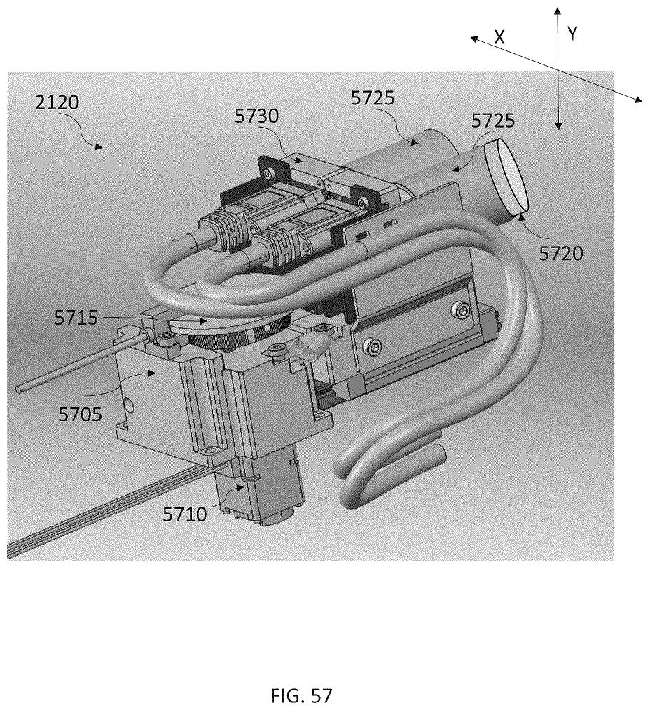

FIG. 56 depicts a recess in a retaining mechanism of a sample handling apparatus described herein in accordance with some example implementations. As shown in FIG. 56, the retaining mechanism 1408, 1422 can include one or more recesses 5605. The recesses 5605 can enable a user to insert or remove a slide more easily into the retaining mechanism 1408 and 1422. With reference to FIG. 14, and the sample handling apparatus 1400, either of the first retaining mechanism 1408 and/or the second retaining mechanism 1422 can include clips configured to hold substrates in place within the first retaining mechanism 1408 or the second retaining mechanism 1422. The first retaining mechanism or second retaining mechanism of the sample handling apparatus 400 or 3500 can also include clips as shown in regard to those configured with respect to the sample handling apparatus 1400. As shown in FIG. 56, the second retaining mechanism 1422 can include one or more clips 5610. The clips 5610 can be secured to the first retaining mechanism 1408 and/or the second retaining mechanism 1422 via a screw or bolt 5620. The clip 5610 can pivot around the bolt 5620 in a defined travel path. A cutout can be configured on a bottom surface (e.g., the surface directly opposite the mounting surface of the retaining mechanism 1422) to limit lateral or pivotal travel of clip 5610 about the bolt 5620 with respect to the surface of the retaining mechanism 1422. The retaining mechanism 1408 or 1422 can include a pin around which the cutout can be arranged so as to limit travel of the clip 5610 when the pin abuts either side of the cutout.

The clip 5610 can include a number of features configured to ease manipulation and grip by a user. For example, the clip 5610 can include a beveled edge extending around the perimeter of the clip 5610 at the upper and/or lower surfaces. The clip 5610 can include a depression 5615 at which a user can apply force with a finger or thumb to cause the end of the clip 5610 to raise such that substrates can be added or removed from the retaining mechanisms 1408 or 1422. In some embodiments, the clip 5610 can be solid. In some embodiments, the clip 5610 can include a cut-out portion. In some embodiments, a profile of the bottom surface of the clip 5610 can have a thinner portion toward the tip of the clip 5610. In this way, a user can more easily maneuver the clip 5610 over the substrate and secure the substrate in the clip 5610 on the retaining mechanism 1408 or 1422.

FIG. 57 depicts a focus motor of a sample handling apparatus described herein in accordance with some example implementations. The focus motor shown in FIG. 57 can correspond to the focus motor 3530 described in relation to FIG. 35. As shown in FIG. 57, the focus motor 5705 can be coupled to an image capture device, such as the image capture device 2120 described herein. The focus motor 5705 can include an actuator 5710 configured to adjust one or more settings of the image capture device 2120.

As further shown in FIG. 57, the focus motor 5705 can include a cam 5715. The cam 5715 can be configured to a mechanical device that can be operatively coupled to the image capture device 2120. The cam 5715 can actuate the image capture device 2120 along a linear axis, such as an X axis as shown in FIG. 57. For example, the cam 5715 can actuate the image capture device in a horizontal manner relative to a field of view, such that actuation of the cam 5715 can cause the image capture device to move from side to side or back and forth relative to an object being imaged. The cam 5715 can include a profile or shape such that when the cam 5715 rotates actuation of the image capture device 2120 is achieved. Advantageously, the cam 5715 can provide finer, more precise control of the actuation of the image capture device 2120 along a linear axis compared to existing actuation devices. In some embodiments, the cam 5715 can actuate a distance between 0 mm and 20 mm, 0 mm and 14 mm, 0 mm and 10 mm, 0 mm and 5 mm. A tolerance of the cam actuation distance can be between 2.0 mm to 2.5 mm.

The focus motor 5705 can be coupled to a spring, such that rotation of the focus motor can cause the spring to extend or retract as the cam 5715 rotates. For example, the spring can be configured to provide about 1.3 lbs of force at a maximal cam position and about 0.9 lbs of force in a minimal cam position.

As further shown in FIG. 57, the image capture device 2120 can include a phase contrast objective 5720. The phase contrast objective 5720 can be configured for use when imaging transparent tissues or tissues which may require Eosin staining to increase contrast of the tissues prior to imaging. Eosin staining can cause reduced data sequencing quality. For example, staining with an Eosin dilution of 1:20 has shown sequencing data quality to be reduced. The use of a phase contrast objective 5720 can advantageously improve contrast of imaged tissues and reduce the time needed to apply contrast stains to tissues in experimental workflows.

The phase contrast objective 5720 can include objectives configured for phase contrast microscopy in dry, dipping, and immersion imaging modalities. The phase contrast objective 5720 can be used in multiple illumination modalities, such as brightfield and epi-fluorescence. In some embodiments, a plurality of phase contrast objectives 5720 can be configured on the image capture device 2120. In some embodiments, the phase contrast objective 5720 can include a magnification between 10× and 30×, a numerical aperture between 0.20 and 0.60, and a working distance between 6.0 mm and 18.0 mm. In some embodiments, the phase contrast objective 5720 can be coupled to the image capture device 2120 via a threaded connection. In some embodiments, the phase contrast objective 5720 can include a condenser phase annulus or a parfocal length extender.

In some embodiments, the image capture device 2120 can include one or more cameras 5725 mounted in a stage or a bracket 5730. In some embodiments, the stage or bracket 5730 can include a unibody construction configured to hold one or more cameras 5725. The unibody construction of the stage 5730 can allow improved serviceability of the image capture device 2120. In some embodiments, printed circuit boards (PCBs) associated with the cameras 5720 can be configured with respect to the shape of the stage 5730 to improve manufacturing quality and image alignment, and to reduce clocking. In some embodiments, one or more electrical components of the sample handling apparatus 400, 1400 described herein can include EMI shielding.

In some embodiments, the camera stage 5730 can be secured to the bottom portion 5315 by a shipping bolt and a hard stop to secure the image capture device 2120 in place during transit.

In some embodiments the cameras 5725 can have a working distance between 56 mm and 60 mm. In some embodiments, a tolerance associated with the working distance can be between +/−0.8 mm to +/−1.95 mm.

### EXAMPLES

### Example 1: Efficient Analyte Capture from Slide-Mounted Fresh Frozen Mouse Brain Sections onto Spatial Array Slides

Analyte capture onto spatially barcoded arrays and subsequent sequencing was demonstrated under sandwich and non-sandwich conditions. For the test (sandwiching) condition, archived tissue-mounted standard glass slides containing hematoxylin/eosin stained fresh frozen mouse brain sections were used. For control (non-sandwich) condition, array substrate slides (e.g., GEx array slides) with hematoxylin/eosin stained fresh frozen mouse brain sections mounted directly onto the array area were used. Under both conditions, tissue sections were subjected to a hematoxylin destaining step. Slides processed according to the “sandwiching” condition were briefly dried at 37° C., then mounted in an instrument along with a GEx slide and a permeabilization buffer comprising sarkosyl and proteinase K. Upon sandwich closure in the instrument, the tissue sections were permeabilized for 1 minute. For the tissue-mounted GEx slides processed according to the non-sandwich condition, sections were permeabilized for 5 minutes using the same permeabilization buffer without sandwiching. For both conditions, following permeabilization, captured polyA-containing mRNA transcripts on the GEx slides were reverse transcribed into cDNA, followed by standard sequencing library preparation and sequencing.

Results depicting median genes per spot and median UMI counts per spot are shown in FIG. 44.

Visual heat map results showing Log 10 UMIs are shown in FIG. 45. Spatial patterns of the Log 10 UMI counts were similar across the sandwich and non-sandwich conditions.

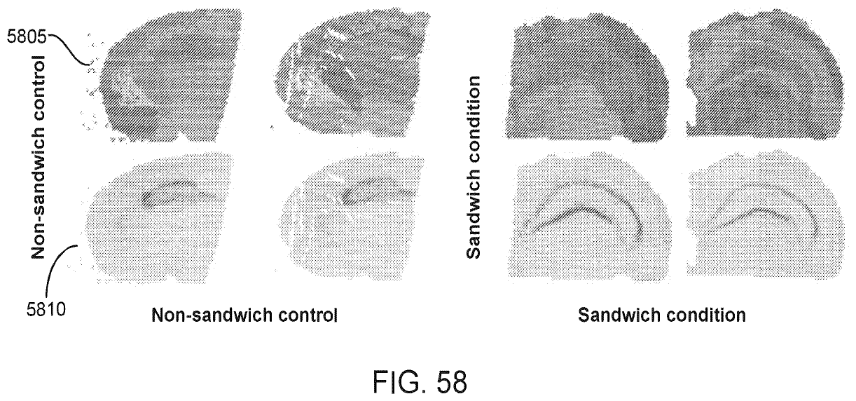

Spatial clustering analysis (top row 5805) and analysis of hippocampal transcript Hpca (bottom row 5810) are depicted in FIG. 58. Spatial patterns were comparable across the sandwich and non-sandwich conditions.

In some embodiments, instead of sandwiching the substrate including a sample of tissue with the substrate including the array of spatial barcodes described herein, the substrate including a sample of tissue can be sandwiched with a tissue optimization assay substrate which has a distributed area containing a plurality of PolyT capture probes. mRNA transcripts can be reverse transcribed in the presence of fluorescently labeled nucleotides, which can result in fluorescent cDNA linked to the capture probes. The linked cDNA can then be imaged. Image brightness and image sharpness can provide indications of the degree to which permeabilization and transcript capture was accomplished.

### Example 2: Sandwich Assembly Using Angled Closure and Closing Speed for Minimal Bubble Generation/Trapping During Permeabilization

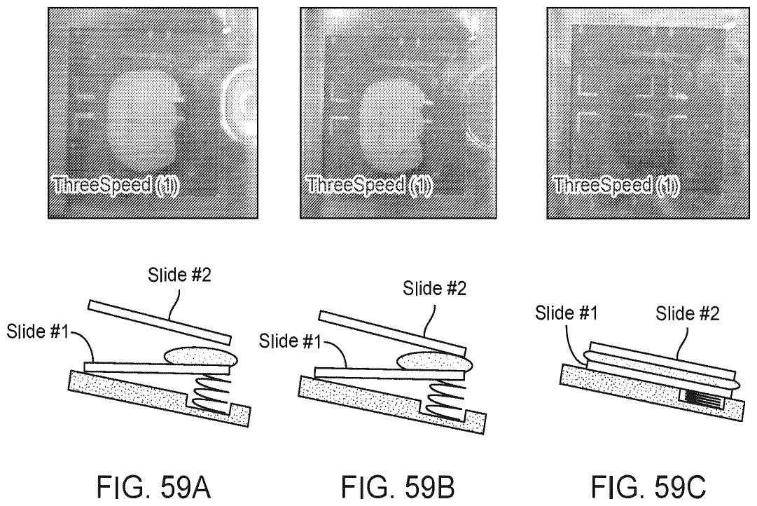

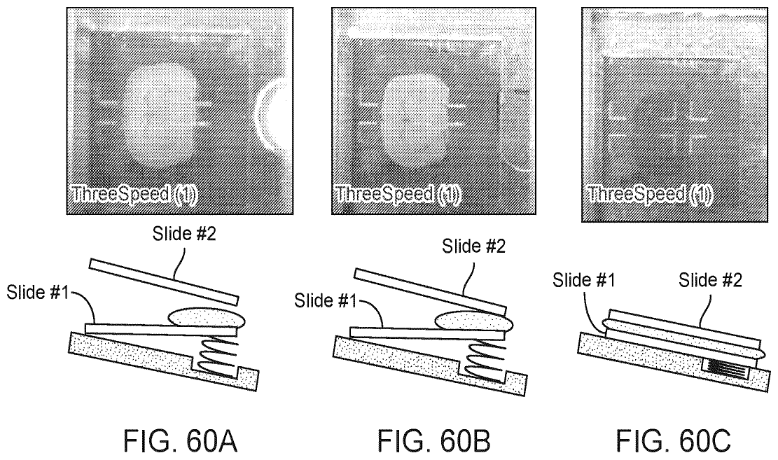

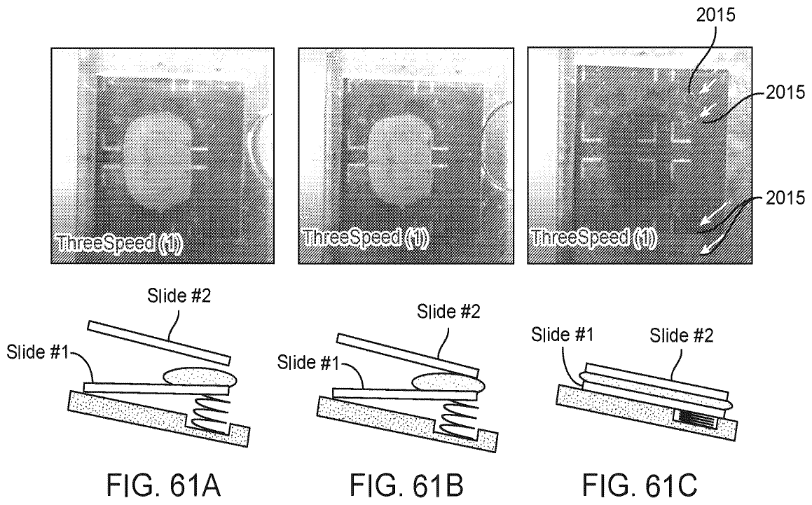

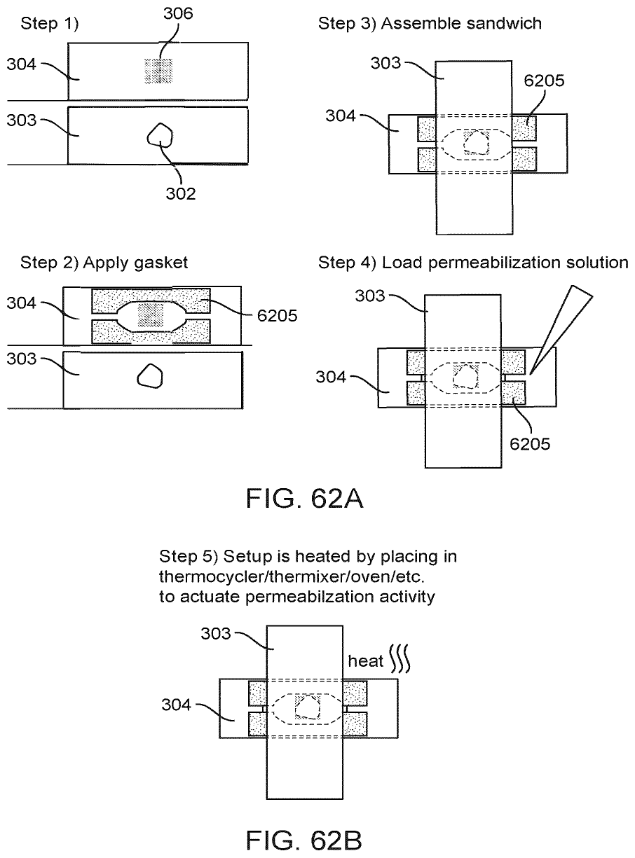

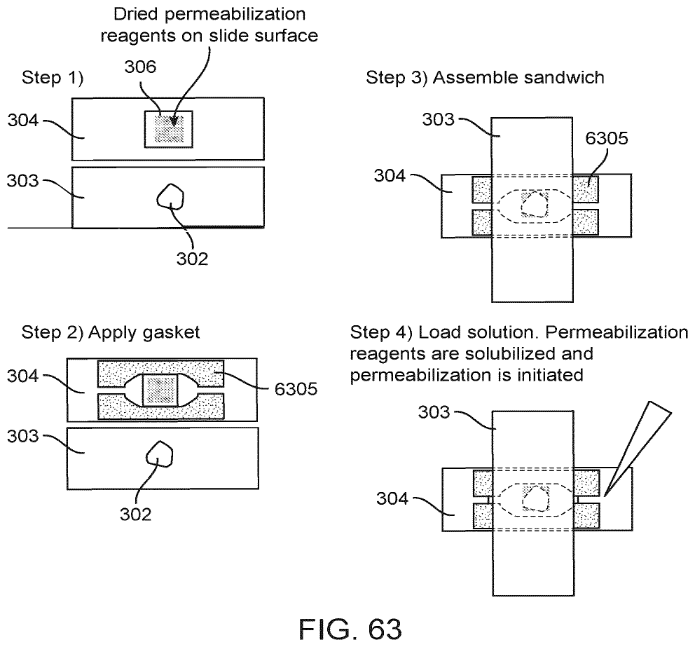

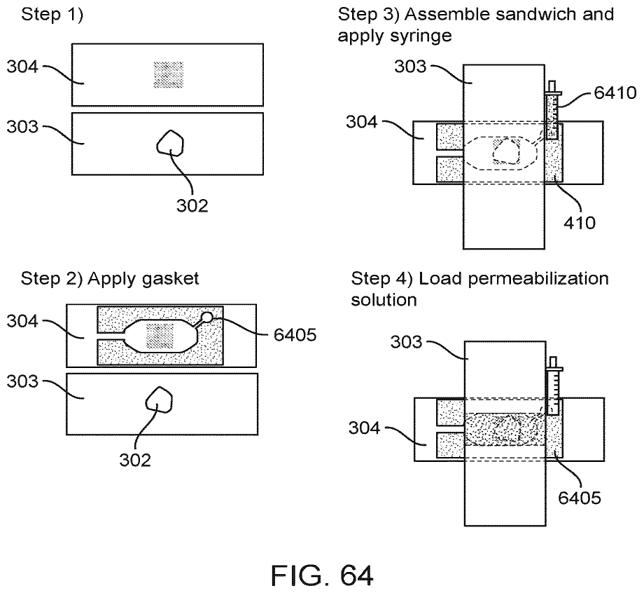

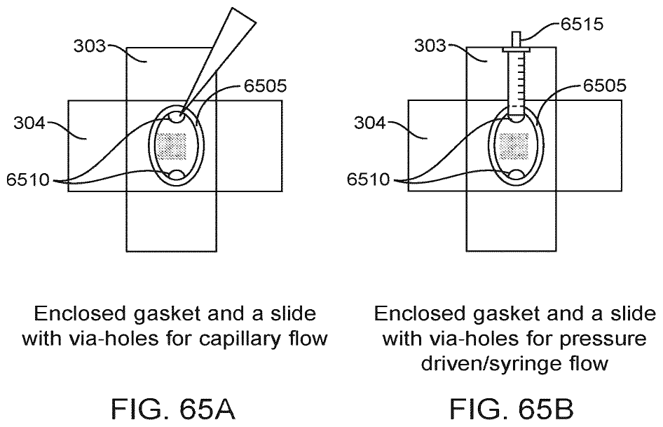

In one example aspect, an angled closure (e.g., see workflow 1700 and FIGS. 17A-18B) for the sandwich configuration was tested at three different closing speeds resulting in closing times of approximately 370 ms (fast), approximately 550 ms (medium), and approximately 1100 ms (slow), respectively. For each of the closing speed conditions, slide 1706 was a glass slide with a coronal mouse brain tissue section mounted thereon and slide 1712 was a GEx slide comprising an array of spatially encoded capture probes. For the fast closing speed condition (e.g., closing occurring within approximately 370 ms), it was observed that a liquid reagent drop (e.g., drop 1705) filled the gap (e.g., gap 307, FIG. 3) between the two substrates without any bubbles trapped within the reagent medium and between the substrates. See FIGS. 59A-59C. For the medium closing speed condition (e.g., closing occurring within approximately 550 ms), it was observed that a liquid reagent drop filled the gap between the two substrates without any bubbles trapped within the reagent medium in between the substrates. See FIGS. 60A-60C. For the slow closing speed condition (e.g., closing occurring within approximately 1100 ms), it was observed that a liquid reagent drop filled the gap between the two substrates with a few bubbles 2015 trapped within the reagent medium and between the substrates. See FIGS. 61A-61C. However, even with the slow closing speed, there were no bubbles observed between the tissue section on slide 1706 and array area on slide 1112. Accordingly, efficient analyte capture is enabled at all three closing speeds, with fast and medium speeds reducing the incidence of bubble trapping anywhere in the sandwich area. The observation that fast and medium closing speeds minimized bubble generation/trapping were consistent across multiple rounds. While example closing speed values are described above, other closing speeds are possible.

### Example 3. Immunostaining in Conjunction with a Sandwich Assembly Workflow

This example provides an exemplary method for integrating immunostaining into a sandwich assembly workflow as described herein. In a non-limiting example, a biological sample is sectioned and placed on a first slide. After fixing (e.g., with 2% formalin or with methanol) and blocking (e.g., with Triton-X), the biological sample is subjected to an antibody incubation step. Following the antibody incubation step, the method further comprises an antibody staining step. The antibody staining step can include a direct method of immunostaining in which a labelled antibody binds directly to the analyte being stained for. Alternatively, the antibody staining step can include an indirect method of immunostaining in which a first antibody binds to the analyte being stained for, and a second, labelled antibody binds to the first antibody. Following the antibody staining step, the sample is imaged, e.g., by immunohistochemistry or immunofluorescence. Following imaging, a second slide comprising a feature array is placed in proximity to the first slide, creating a sandwich configuration. Permeabilization occurs while the slides are in the sandwich configuration.

Exemplary permeabilization conditions can include permeabilization with pepsin, or permeabilization with proteinase K and SDS. Analytes migrate to and are captured by probes on the second slide, then extended to capture the complementary sequence of the captured oligonucleotides and analytes. Following permeabilization, the sandwich is disassembled and the extended capture probe is then amplified and sequenced according to any one of the methods described herein. Subsequent sequence analysis is then used to determine spatial information regarding the analytes captured from the tissue sample.

### Example 4: Additional Embodiments

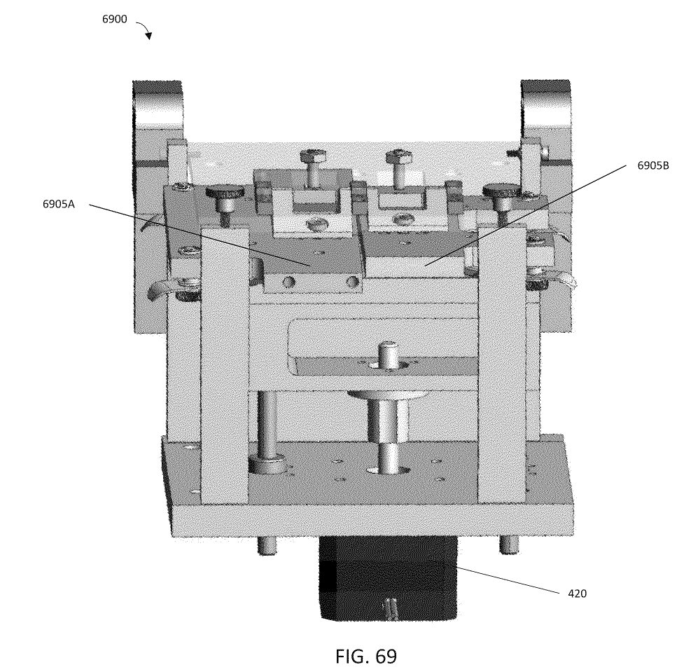

In some aspects, alignment of the first substrate and the second substrate may be improved with an articulating first member 404. FIG. 69 is a front view of an example sample handling apparatus 6900 in accordance with some example implementations. As shown, the sample handling apparatus 6900 includes independent first members 6905A and 6905B. In some aspects, the first members 6905A and 6905B may be configured to independently move/articulate (e.g., in three directions or the like). The independent movement of the first members 6905A and 6905B may be beneficial to compensate for any differences in thickness of substrates (e.g., slides 303) and/or tissue samples (e.g., sample 302) retained in the first members 6905A and 6905B. The independent movement may also maintain a uniform gap height or separation distance between a first substrate and a second substrate (e.g., first substrate 303 and second substrate 304 in a sandwich configuration). In some aspects, the first members 6905A and/or 6905B may include an array position indicator (not shown) to aid in an x-y alignment between substrates (e.g., first substrate 303 and second substrate 304 in a sandwich configuration).

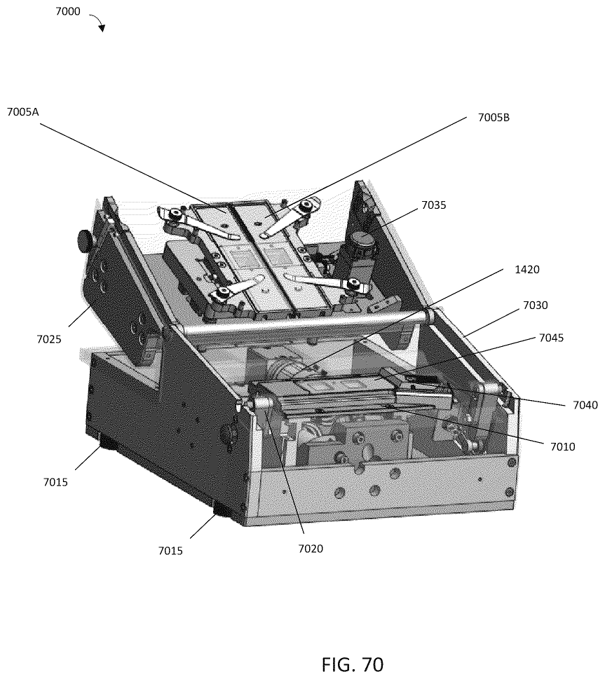

In some aspects, alignment of the first substrate and the second substrate may be improved with a fixed second member 410. FIG. 70 is a perspective view of an example sample handling apparatus 7000 in accordance with some example implementations. As shown, the sample handling apparatus 7000 includes independent first members 7005A and 7005B, the image capture device 1420, and a second member 7010. In some aspects, the first members 7005A and 7005B may be coupled to a linear actuator (e.g., the linear actuator 420) configured to move the first members 7005A and/or 7005B along a z axis orthogonal to a plane of the fixed second member 7010.

In some aspects, the movement of the first members 7005A and/or 7005B may occur after a user manually closes the first members 7005A and/or 7005B over the second member 7010. As further shown, the image capture device 1420 may be positioned inferior to (e.g., below) the second member 7010. As further shown, the image capture device 1420 may be positioned inferior to (e.g., below) the second member 7010. This position may allow the image capture device 1420 to capture consistent in-focus images of the first substrate 303, the tissue sample 302, the second substrate 304, and/or the capture probes 306 at different time periods of a sandwich configuration analysis. In some aspects, a lighting source may positioned on, or superior to (e.g., above) the first members 7005A and/or 7005B to aid in image capture. Further, the fixed second member 7010 may allow the reagent droplet (e.g., permeabilization solution 305) to remain on the second substrate 304 more easily than if the second member articulated and/or changed angles. In some aspects, moving the first members 7005A and/or 7005B along the z axis as opposed to the second member 7010 may allow single cell analysis and a thinner spacer (e.g., spacers 3610, 6805). In some aspects, an array indicator (not shown) may be disposed on the first member 7005A and/or 7005B and/or the second member 7010.

The array indicator may be installed using laser engraving.

As further shown in FIG. 70, the sample handling apparatus 7000 also includes a plurality of adjustable feet 7015, which may be adjusted to level the sample handling apparatus relative to a surface on which it is located. The sample handling apparatus 7000 can also include a lock 7020 to constrain or secure the top portion 7025 relative to the bottom portion 7030 (e.g., when the two portions are closed together relative to one another to form a sandwich configuration as described herein). As further shown in FIG. 70, the sample handling apparatus 7000 includes a motor 7035 to drive the first members 7005A and 7005B vertically (e.g., downward) relative to the second member 7010. The sample handling apparatus 7000 also includes a clip 7040 configured to constrain the second substrate 7045 within the second member 7010. The clip 7040 can constrain movement of the second substrate 7045 in planes of motion associated with x-, y-, and z-axes of a Cartesian coordinate system.

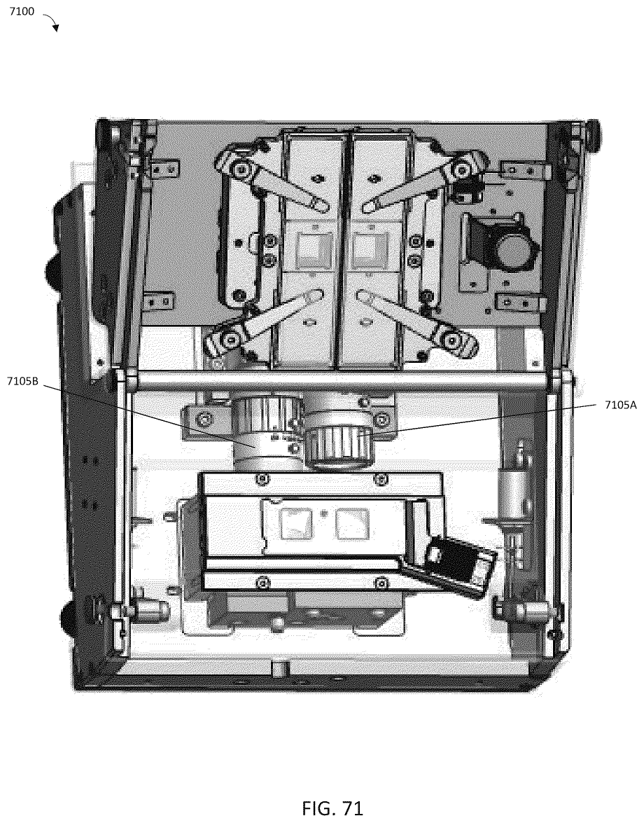

FIG. 71 is a perspective view of a sample handling apparatus 7100 including multiple image capture devices in accordance with some example implementations. A non-limiting number of embodiments of sample handling apparatuses described herein can include multiple image capture devices. As shown in FIG. 71, in some embodiments, the sample handling apparatus 7100 can include more than one image capture devices. For example, the sample handling apparatus 7100 includes a first image capture device 7105A and a second image capture device 7105B, each of which can correspond to image capture device 1420 described in relation to FIG. 14.

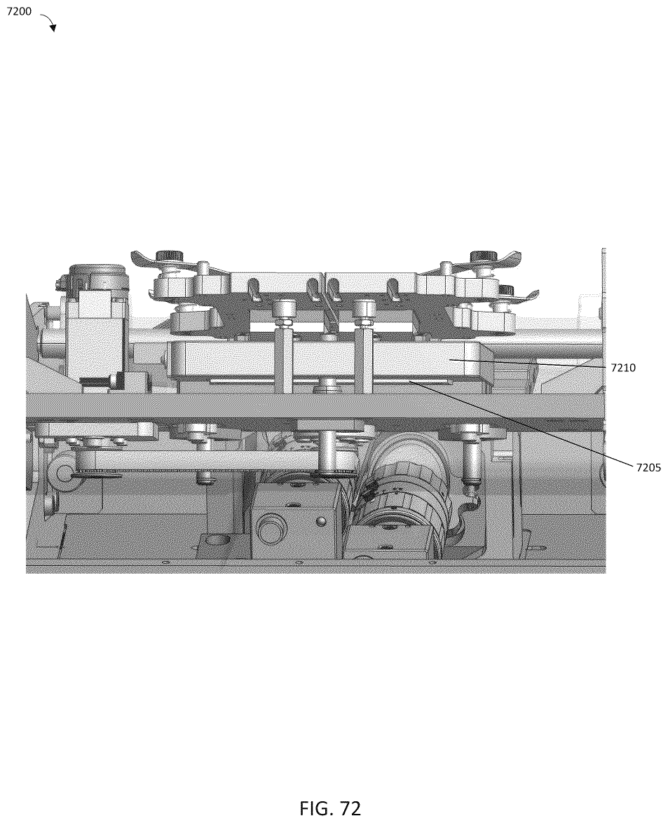

FIG. 72 is a perspective view of a portion 7200 of the sample handling apparatus described herein including a light emitting diode (LED) in accordance with some example implementations. In some embodiments, multiple diodes can be used, for example one for each color. The sample handling apparatus portion 7200 shown in FIG. 72 is shown in a closed or sandwich configuration. The LED illumination source 7205 can be configured in an inferior position relative to the second member 7210. For example, the LED 7205 is shown in FIG. 72, below the second member 7210. The second member 7210 can correspond to the second member 7010 described in relation to FIG. 70 and the second member 410 described in relation to FIG. 4 and elsewhere herein. In some embodiments, the LED 7205 can be operatively controlled by a data processor configured within the sample handling apparatus described herein. For example, processor 4620 described in relation to FIG. 46 can execute computer readable instructions stored in memory 4626 and/or the sample handling apparatus software building block 4700 described in relation to FIG. 47 to control operation and illumination of the LED 7205.

In some embodiments, the sample handling apparatus described herein can include components and functionality configured to provide accurate control and positioning of the top portion of the sample handling apparatus (e.g., the top portion 7025 described in relation to FIG. 70) relative to the bottom portion of the sample handling apparatus (e.g., the bottom portion 7030 described in relation to FIG. 70) as the top portion is brought into proximity and contact with the bottom portion during formation of the closed or sandwich configuration described herein. In embodiments of the sample handling apparatuses described herein, optical homing can be performed by acquiring sensor data and processing the sensor data to determine a location of the top portion relative to the bottom portion so that a closed, unclosed, or an incompletely closed configuration of the sample handling apparatus can be determined. In some embodiments, a motor can be configured to control closure of the top portion relative to the bottom portion of the sample handling apparatus. Data acquired via a sensor coupled to the motor data can be used to determine a location of the top portion relative to the bottom portion so that a closed, unclosed, or an incompletely closed configuration of the sample handling apparatus can be determined. A variety of other non-limiting components can be included in some embodiments of the sample handling apparatus described herein to perform the optical homing by controlling the closure of the top portion and bottom portion of the sample handling apparatus. In some embodiments, determining the location of the top portion relative to the bottom portion can be performed using electro-mechanical sensing methods. For example, determining that the sample handling apparatus is in a closed, unclosed, or an incompletely closed configuration can be achieved using motor sensors. Using these methods, the sample handling apparatus described herein can advantageously mitigate incomplete closures of the top portion and bottom portion during formation of the closed or sandwich configuration. A variety of embodiments of the sample handling apparatuses described herein can be configured to perform optical and/or electro-mechanical homing without limitation.

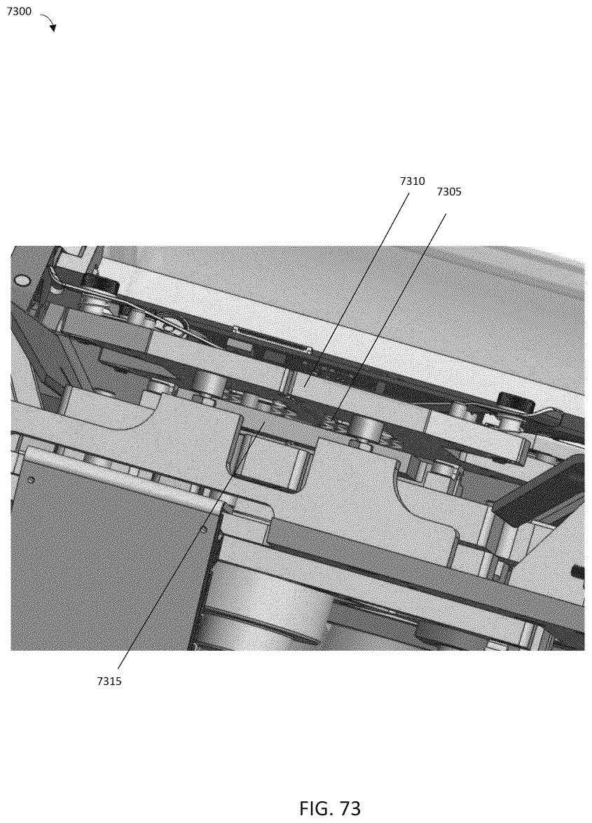

FIG. 73 is a perspective view of a portion 7300 of the sample handling apparatus described herein including a spring configured to aid closure of the sample handling apparatus in accordance with some example implementations. For example, as shown in FIG. 73, the portion 7300 shows the sample handling apparatus in a closed or sandwich configuration. A spring 7305 can be configured between a first member 7310 (corresponding to first member 7005A or 7005B described in relation to FIG. 70) and second member 7315 (corresponding to the second member 7010 described in relation to FIG. 70 and the second member 410 described in relation to FIG. 4 and elsewhere herein). The spring 7305 can absorb excess force during closure and can improve the consistent application of closure forces over the lifetime of the sample handling apparatus. The spring 7305 can also reduce vibration sensitivity and minor motor movements within the sample handling apparatus described herein. For example, vibration and minor motor movements may cause bubbles to be formed during the closed or sandwiched configuration, which can adversely affect permeabilization and imaging techniques when performing spatial assays described herein.

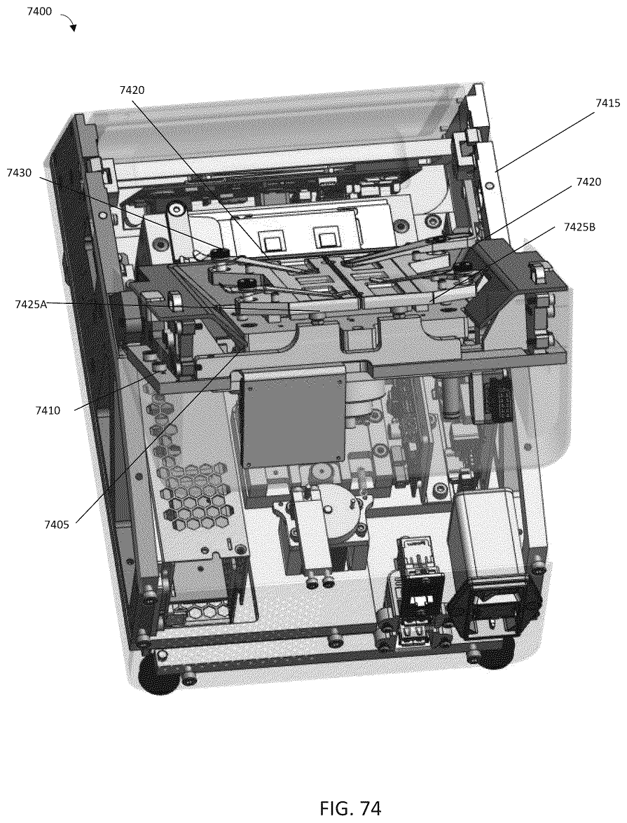

FIG. 74 is a perspective view of a portion of a sample handling apparatus 7400 including a sensor 7405 in accordance with some example implementations. As shown in FIG. 74, the top-down perspective view of the portion 7400 shows the sample handling apparatus in an open configuration. The top portion 7410 of the sample handling apparatus 7400 can include the sensor 7405. In some embodiments, the sensor 7405 can include an optical beam-break sensor for use in performing optical homing as the top portion 7410 is closed relative to the bottom portion 7415. As an optical sensor, the sensor 7405 can emit an optical beam and can detect when the beam has been broken, such as when the beam is broken by a corresponding component in the bottom portion 7415. In this way, a “home” position of the top portion 7410 of the sample handling apparatus 7400 relative to the bottom portion 7415 can be determined. It may be desirable to determine a position beyond the “home” position, for example, to “park” or rest the top portion 7410 in the sample handling apparatus 7400, such as when the top portion 7410 is completely opened. In the “park” or rest position, a user can be provided with a more rigid configuration of the top portion 7410 to allow placement of substrate slides within the substrate receiving clips 7420 coupled to the first members 7425A and 7425B via a screw 7430. The substrate receiving clips 7420 can include features to reduce the chance of a user's glove or skin becoming pinched between the substrate receiving clip 7420 and the screw 7430.

In some embodiments, the sensor 7405 can include a trigger feature covering an aperture of the sensor 7405. The trigger feature can be configured as portion of a frame of the bottom portion 7415. The trigger feature can enable the sensor 7405 to sense and thus trigger transmission of sensor data indicative of movement of the top portion 7410 relative to the bottom portion 7415. In this way, early or premature triggering of the sensor 7405 can be reduced. In some embodiments, the sensor 7405 can be a reed sensor configured in a frame of the top portion 7410. As a reed sensor, the sensor 7405 can engage with a magnet configured in a frame of the bottom portion 7415. The configuration of the reed sensor and the magnet can reduce early or premature triggering of the sensor 7405 as the top portion 7410 moves relative to the bottom portion 7415.

In some embodiments, the “home” position of the top portion 7410 of the sample handling apparatus 7400 can be additionally or alternatively determined using an impedance sensor coupled to a motor controlling the closure of the top portion 7410 with respect to the bottom portion 7415. The impedance sensor can measure the impedance of the motor at the start of closing the top portion 7410 toward the bottom portion 7415 and can assign a “zero” value, corresponding to the “home” position when the top portion 7410 contacts a physical bump or stop configured in the bottom portion 7415 In this way, the “zero” value can correspond to the “home” position and articulation of the top portion 7410 via the motor can be performed relative to the “zero” value.

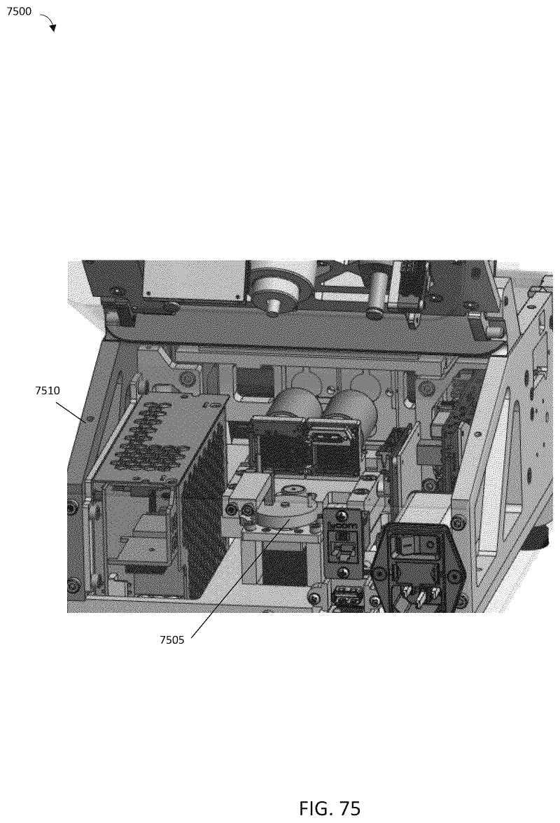

FIG. 75 is a perspective view of a portion 7500 of a sample handling apparatus as described herein including a motor 7505 in accordance with some example implementations. As shown in FIG. 75, the portion 7500 is shown as rear perspective view of the sample handling apparatus without a panel or a cover attached to the bottom portion 7510 to provide greater clarity of the motor 7505. The motor 7505 can be a focus motor configured to actuate and control movement of the image capture device(s) 1420, 7105A and/or 7105B described in relation to FIGS. 14 and 71, respectively, relative to the second member 7010 of FIG. 70. In this way, the motor 7505 can actuate so as to allow the sample handling apparatus accommodate array substrates of varying thicknesses. In some embodiments, the bottom portion 7510 can include an antenna attached to a frame element of the bottom portion 7510. In some embodiments, the antenna can be configured behind a rear panel of the bottom portion 7510. For clarity illustrating the motor 7505, the rear panel of the bottom portion 7510 has been removed.

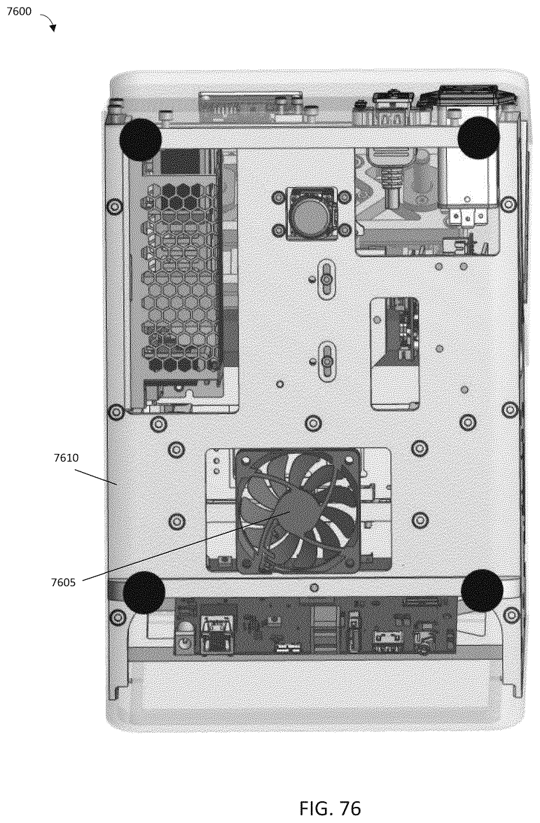

FIG. 76 is a bottom view of a sample handling apparatus 7600 including a fan in accordance with some example implementations. The fan 7605 can be configured in a base 7610 of the sample handling apparatus 7600. The fan 7605 can be operatively controlled to provide cooling to the sample holder 7600 before, during, and after use thereof.

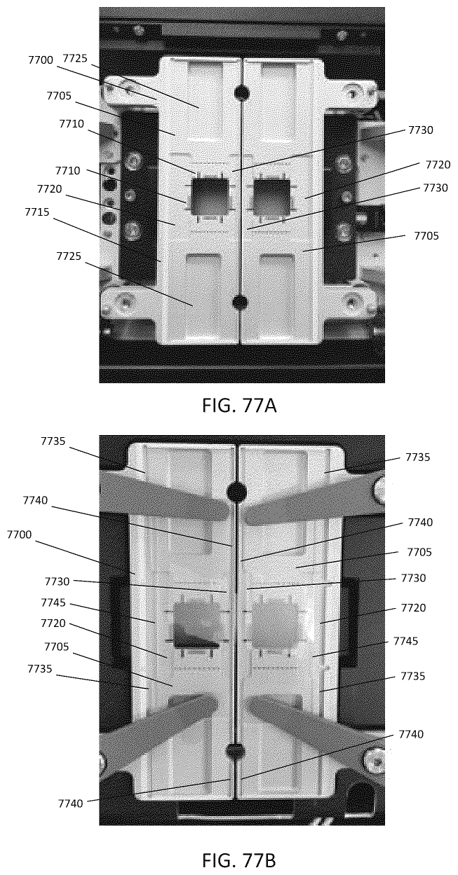

FIG. 77A is an image illustrating an embodiment of a retaining mechanism configured for use in the sample handling apparatus 400, 1400, and 3400 described herein. The retaining mechanism 7700 described in relation to FIG. 77A can correspond to the retaining mechanism 1408 (e.g., the first retaining mechanism) described in relation to FIGS. 14, 48B, 49B, and 56. As shown in FIG. 77A, the retaining mechanism 7700 can include a first surface 7705. The first surface 7705 can include an array area indicator 7710. Advantageously, when a substrate, such as substrate 1406 including a biological sample, is placed in the retaining mechanism 7700, the sample may be overlaid atop the array area indicator 7710. The markings, design, and dimensions of the array area indicator 7710 can vary. A wall 7715 can surround portions of the first surface 7705 and can secure the substrate 1406 within the retaining mechanism 7700. A first recess 7720 can be provided at a first depth into the first surface 7705. The first recess 7720 can abut the array area indicator 7710. In some embodiments, a second substrate 1422 can also include a spacer surrounding the array, such as spacer 507, 3610, or 6805 described herein. In this way, when the biological sample or portion thereof is vertically aligned and fluidically coupled via the reagent medium with the array, the first substrate 1406, the second substrate 1412, and the spacer 507, 3610, or 6805 form a substantially enclosed chamber retaining the reagent medium.

As further shown in FIG. 77A, the retaining mechanism 7700 can also include substrate handling recesses 7725. The substrate handling recesses 7725 can allow a user to manually load or remove a substrate into the retaining mechanism 7700 more easily. The retaining mechanism 7700 can also include a second recess 7730 formed into the first surface 7705 at second depth. The second recess 7730 can abut the array area indicator 7715. In some embodiments, the second depth can be equal to or greater than the first depth of the first recess 7720. In some embodiments, the second depth is shallower than the first depth.

FIG. 77B is an image illustrating another embodiment of the retaining mechanism 7700 described in FIG. 77A. As shown in FIG. 77B, the retaining mechanism 7700 can also include a first channel 7735 that can be in fluidic communication with the first recess 7720. The first channel 7735 can be formed into the first surface 7705 at a third depth. In some embodiments, the third depth can be equal to or greater than the first depth of the first recess 7720. In an alternative embodiment, the third depth is shallower than the first depth. As further shown, the retaining mechanism 7700 can also include a second channel 7740. The second channel 7740 can be in fluidic communication with the second recess 7730. The second channel 7740 can be formed into the surface 7705 of the retaining mechanism 7700 at a fourth depth. The fourth depth can be equal to or greater than the second depth of the second recess 7730. In an alternative embodiment, the fourth depth is shallower than the second depth. As further shown in FIG. 77B, the first recess 7720 can include an elevated ridge 7745 extending through the first recess 7720. The elevated ridge can be formed to a ridge height. The elevated ridge 7745 can confine fluid within the first recess 7720. In some embodiments, the second recess 7730 can also include an elevated ridge to confine fluid within the second recess.

Advantageously, the first channel 7730 and/or the second channel 7740 can be sized, positioned, and formed at a depth to prevent and/or reduce capillary flow during a sandwiching process disclosed herein, e.g., when the substrate 1406 and a second substrate, such as substrate 1412, are brought into a sandwich configuration.

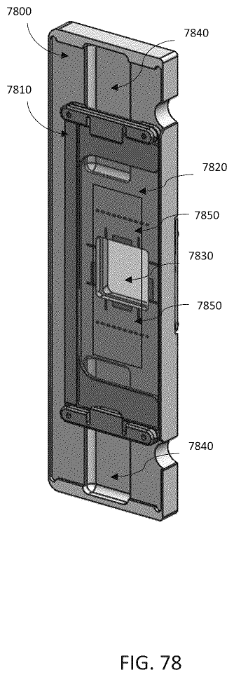

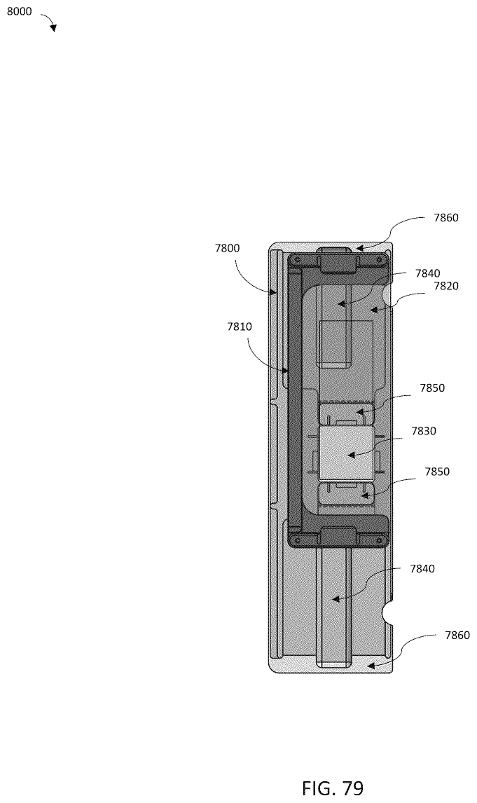

FIG. 78 is a perspective view of a retaining mechanism and a frame member in accordance with some example implementations. The retaining mechanism 7800 described in relation to FIG. 78 can correspond to the retaining mechanism 1408 (e.g., the first retaining mechanism) described in relation to FIGS. 14, 48B, 49B, 56, 77A, and 77B. As shown in FIG. 78, a retaining mechanism 7800 can be configured to receive and couple (e.g., removably couple) with a frame member 7810. The retaining mechanism 7800 can include a plurality of magnets arranged within and/or around a periphery of the retaining mechanism 7800. A variety of patterns and distributions of the magnets within the retaining mechanism 7800 can be envisioned. The frame member 7810 can couple with the retaining mechanism 7800. In some embodiments, the frame member 7810 can be coupled (e.g., removably coupled) to the retaining mechanism 7800 via the plurality of magnets. The frame member 7810 can be configured to receive a substrate 7820, such as a substrate including a biological sample. As shown in FIG. 78, the substrate 7820 can be a transparent substrate and features on the top portion of the retaining mechanism 7800 can viewed through the substrate 7820. The frame member 7810 can include a ferromagnetic material to aid coupling with the plurality of magnets in the retaining mechanism 7800. The retaining mechanism 7800 can include a viewing window 7830. The frame member 7810 can be excluded from the viewing window 7830 when the frame member 7810 is coupled to the retaining mechanism 7800. Image data of the sample and/or the array can be acquired by an image capture device of the sample holder described herein configured with a field of view corresponding to the viewing window 7830. The retaining mechanism 7800 can include recessions 7840 to allow a user to manipulate the frame member 7810. The retaining mechanism 7800 can also include a support area 7850 surrounding the viewing window 7830. As shown in FIG. 79, the retaining mechanism 7800 can also include stops 7860 configured to abut the frame member 7810 when the frame member 7810 is received within the retaining mechanism 7800. While FIG. 79 shows at least one stop 7860 abutting the frame member 7810, it is understood that the frame member 7810 can be coupled to the retaining mechanism 7800 such that it does not abut any of the stops 7860. As shown in FIG. 79 the retaining mechanism 7800 can include a smaller support area 7850 and narrower recessions 7840 than those shown in FIG. 78.

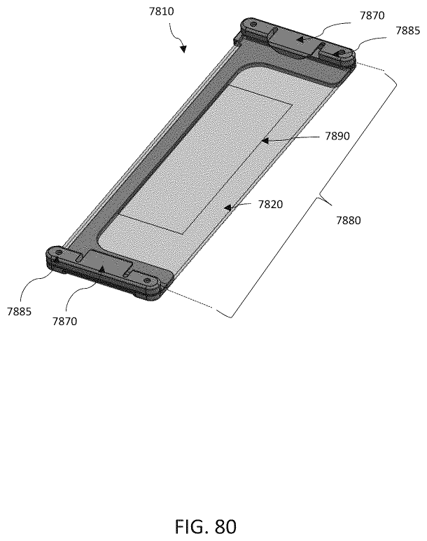

FIG. 80 is a perspective view of the frame member of FIG. 78 in accordance with some example implementations. As shown in FIG. 80, the frame member 7810 can include clips 7870. The clips 7870 can help secure the substrate 7820 within the frame member 7810. The frame member can include an opening 7880 at which the substrate 7820 can be inserted into or removed from the frame member 7820. The opening 7880 can be formed between frame member segments 7885 of the frame member 7810. The frame The substrate 7820 can also include a viewing area indicator 7890. The shape and size of the viewing area indicator 7890 can correspond to a shape and size of the viewing area 7830 of the retaining mechanism 7800.

In some embodiments, a member of a sample handling apparatus disclosed herein comprises a retaining mechanism assembly in accordance with some example implementations. The retaining mechanism assembly can include a frame coupled to a retaining mechanism. The retaining mechanism can correspond to a first retaining mechanism 1408 (e.g., the first retaining mechanism) described in relation to FIGS. 14, 48B, 49B, 56, and 78. The frame can be pivotably mounted within a member of the sample holder as described herein, such as the first member 1404, via a rotational element. The rotational element can allow the frame and the retaining mechanism to pivot with respect to a horizontal surface on which the sample holder is positioned.

The frame can include a first surface. An opening can extend between the first surface and a second surface of the frame. The retaining mechanism can be coupled to the second surface of the frame. The first surface can include a plurality of protrusions on opposite sides of the opening. The protrusions can have a height configured to limit pivotal travel of the frame relative to a horizontal surface on which the sample holder is positioned. In some embodiments, the protrusions can have a height configured to limit the pivotal travel to +4 degrees to −4 degrees. In some embodiments, each of the protrusions can have the same height. In some embodiments, a first protrusion on one side of the opening can have a first height and a second protrusion on a second side of the opening, opposite to the first side, can have a second height that is more or less than the first height.

The frame can include a plurality of recesses. The plurality of recesses can include a plurality of holes through which a plurality of attachment means can extend through the holes and into the retaining mechanisms. In some embodiments, the attachment means can include at screws, nails, dowels, plugs, or the like.

In some embodiments, the retaining mechanism assembly further includes a plurality of brackets. The brackets can receive the rotational element therein to enable the frame to pivotally rotate. The retaining mechanism assembly can also include a frame mount that can be mated to the first surface via the brackets. The protrusions can contact the frame mount to limit the pivotal travel.

The retaining mechanism assembly can also include a plurality of force transfer elements. The force transfer elements can be coupled to the frame and/or the frame mount. The force transfer elements can provide or receive forces generated with respect to pivotal movement of the frame about the rotational element. In some embodiments, the force transfer elements can include compression springs, extension springs, compressible materials, or the like. The arrangement of the plurality of force transfer elements and a corresponding arrangement of a plurality of frame receiver holes at which the plurality of force transfer elements mate with the frame mount can be configured so as to balance a first compression force applied to the frame in a first vertical direction and a second compression force applied to the frame in a second vertical direction. The first vertical direction can be opposite the second vertical direction. In this way, the retaining mechanism assembly can be maintained in a parallel arrangement with respect to a surface on which the sample holder is positioned and deflection forces that may be applied to the frame during loading or removing of substrates within the retaining mechanism can be minimized. In some embodiments, the frame mount includes a plurality of frame receiver holes. The plurality of force transfer elements can be received within the plurality of frame receiver holes.

The subject matter described herein may be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations may be provided in addition to those set forth herein. For example, the implementations described above may be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

One or more aspects or features of the subject matter described herein may be realized in digital electronic circuitry, integrated circuitry, specially designed ASICs, field programmable gate arrays (FPGAs) computer hardware, firmware, software, and/or combinations thereof. These various aspects or features may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which may also be referred to as programs, software, software applications, applications, components, or code, include machine instructions for a programmable processor, and may be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium may store such machine instructions non-transitorily, such as for example as would a non-transient solid-state memory or a magnetic hard drive or any equivalent storage medium.

The machine-readable medium may alternatively or additionally store such machine instructions in a transient manner, such as for example, as would a processor cache or other random access memory associated with one or more physical processor cores.

To provide for interaction with a user, one or more aspects or features of the subject matter described herein may be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices may be used to provide for interaction with a user as well. For example, feedback provided to the user may be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

What is claimed is:

1. A method of capturing analytes from a biological sample, the method comprising:
a) mounting a first substrate on a first member of a sample handling device, the first substrate comprising the biological sample disposed thereon;
b) mounting a second substrate on a second member of the sample handling device, the second substrate comprising an array of capture probes, wherein a first capture probe of the array of capture probes comprises a first spatial barcode sequence and a first capture domain and wherein a second capture probe of the array of capture probes comprises a second spatial barcode sequence and a second capture domain;
c) applying a first reagent medium to the first substrate and/or the second substrate, the first reagent medium configured to release a first analyte from the biological sample;
d) moving the first member and/or the second member such that a first area of the biological sample and the first capture probe are fluidically coupled via the first reagent medium, thereby releasing the first analyte from the first area of the biological sample, wherein the released first analyte binds to the first capture domain;
e) moving the first member and/or the second member to fluidically decouple the first area of the biological sample and the first capture probe;
f) applying a second reagent medium to the first substrate and/or the second substrate, the second reagent medium configured to release a second analyte from the biological sample; and
g) moving the first member and/or the second member such that the first area of the biological sample and the second capture probe are fluidically coupled via the second reagent medium, thereby releasing the second analyte from the first area of the biological sample, wherein the released second analyte binds to the second capture domain.

2. The method of claim 1, wherein the first spatial barcode sequence and the second spatial barcode sequence are identical.

3. The method of claim 2, wherein (d), (e), and (g) are performed with aid of a mechanical fixture provided within the sample handling device, the mechanical fixture configured to maintain alignment of first substrate and the second substrate such that the first area of the biological sample is vertically aligned with a first area of the array during (d), (e), and (g), the first area comprising the first capture probe and the second capture probe.

4. The method of claim 3, wherein the mechanical fixture is manually adjustable.

5. The method of claim 3, wherein the mechanical fixture is adjustable via a controller of the sample handling device.

6. The method of claim 1, wherein the first spatial barcode sequence and the second spatial barcode sequence are different.

7. The method of claim 6, wherein a first area of the array comprises the first capture probe and wherein a second area of the array comprises the second capture probe, and wherein the method further comprises:
acquiring, responsive to (d), first image data comprising a first overlay of the first area of the biological sample with the first area of the array;
acquiring, responsive to (g), second image data comprising a second overlay of the first area of the biological sample with the second area of the array within the capture domain;
registering the first image data and the second image data; and
generating an aligned imaged based on the registering, the aligned image comprising an overlay of the first area of the array with the second area of the array.

8. A system for aligning a sample area with an array area, the system comprising:
a sample holder comprising a first member comprising a first retaining mechanism configured to retain a first substrate, the first substrate comprising a sample disposed on the sample area;
a second member comprising a second retaining mechanism configured to retain a second substrate received within the second retaining mechanism, the second substrate comprising (i) an array of capture probes disposed on the array area and (ii) a reagent medium;
an alignment mechanism configured to move the first member and/or the second member such that the biological sample or a portion thereof is vertically aligned and fluidically coupled via the reagent medium with the array when the first substrate and the second substrate are retained in the first and second members, respectively; and
an image capture device operatively coupled to the sample holder and configured to generate image data of the first substrate and the second substrate within the sample holder, the image capture device comprising a phase contrast objective.

9. The system of claim 8, further comprising
a computing device communicatively coupled to the image capture device and to the sample holder, the computing device comprising a display, a data processor, and a non-transitory computer readable storage medium storing computer readable and executable instructions, which when executed cause the data processor to move the first member and/or the second member such that the biological sample or a portion thereof is vertically aligned and fluidically coupled via the reagent medium with the array when the first substrate and the second substrate are retained in the first and second members, respectively.

10. The system of claim 9, wherein the computer readable and executable instructions, which when executed, further cause the data processor to generate image data of the first substrate and the second substrate when the biological sample or a portion thereof is vertically aligned and fluidically coupled via the reagent medium.

11. A method, comprising:
a) mounting a first substrate on a first member of a sample holder, the first substrate comprising a biological sample disposed thereon, and the sample holder comprising
a first member comprising a first retaining mechanism configured to retain the first substrate,
a second member comprising a second retaining mechanism configured to retain a second substrate received within the second retaining mechanism, the second substrate comprising (i) an array of capture probes disposed on the array area, and (ii) one or more array fiducials,
an alignment mechanism configured to move the first member and/or the second member such that the biological sample or a portion thereof is vertically aligned and fluidically coupled via the reagent medium with the array when the first substrate and the second substrate are retained in the first and second members, respectively, and
an image capture device operatively coupled to the sample holder, the image capture device configured to generate image data of the first substrate and the second substrate within the sample holder, the image capture device comprising a phase contrast objective;
b) mounting a second substrate onto the second member of the sample holder;
c) applying a reagent medium to the first substrate and/or the second substrate, the reagent medium configured to release one or more analytes from the biological sample;
d) using the alignment mechanism to move the first member and/or the second member such that the biological sample or a portion thereof is vertically aligned and fluidically coupled via the reagent medium with the array;
e) responsive to (d), using the image capture device to obtain an array image of (i) an overlay of the biological sample with the array and (ii) the one or more array fiducials.

12. The method of claim 11, wherein the biological sample does not comprise an eosin stain.

13. The method of claim 11, wherein the method further comprises:
receiving, by a data processor, array image data from the image capture device, the array image data comprising the array image of the overlay of the biological sample with the array and the one or more array fiducials;
receiving, by the data processor, sample image data comprising a sample image of the biological sample;
registering, by the data processor, the sample image to the array image by aligning the sample image and the array image;
generating, by the data processor, an aligned image based on the registering, the aligned image comprising an overlay of the sample image with the one or more array fiducials; and
providing, by the data processor, the aligned image.

14. The method of claim 11, wherein the aligned image further comprises the one or more array fiducials aligned with the sample.

15. The method of claim 11, wherein the sample image is of the sample on the first substrate.

16. The method of claim 11, wherein the one or more array fiducials are located on the second substrate adjacent to, within, or distant from the array of capture probes configured on the second substrate.

17. The method of claim 11, wherein the one or more array fiducials surround the array of capture probes.

18. The method of claim 11, wherein the first substrate comprises one or more sample fiducials.

19. The method of claim 11, wherein the array image is acquired such that a portion of the array overlays a portion of the sample based on a location of the one or more array fiducials and/or the one or more sample fiducials.

20. The method of claim 11, wherein the sample image has a higher resolution than the array image.
